(19) United States
(12) Patent Application Publication
(10) Pub. No.: US 2015/0284341 A1
(43) Pub. Date: Oct. 8, 2015

(54) FLUOROALKYL-SUBSTITUTED DERIVATIVES OF PYRIDINE, PYRIMIDINE, AND PYRAZINE

(71) Applicant: Catylix, Inc., Burbank, CA (US)

(72) Inventors: James David Rozzell, Burbank, CA (US); John F. Hartwig, Berkeley, CA (US)

(21) Appl. No.: 14/246,384

(22) Filed: Apr. 7, 2014

(51) Int. Cl. C07D 241/24; C07D 213/79; C07D 213/82; C07D 213/75; C07D 241/18; C07D 239/36; C07D 239/30; C07D 239/42; C07D 241/20; C07D 213/61; C07D 213/85

(52) U.S. Cl. CPC C07D 241/24; C07D 213/61; C07D 213/79; C07D 213/82; C07D 213/75; C07D 213/85; C07D 239/36; C07D 239/30; C07D 239/42; C07D 241/20; C07D 241/18

(57) ABSTRACT

Fluoroalkyl-substituted, nitrogen-containing, aryl heterocyclic compounds are provided. The compounds are derivatives of pyridine, pyrimidine, or pyrazine, and have two or three functional groups bonded to the heterocyclic ring, including a perfluoroethyl, perfluoropropyl, perfluoroisopropyl, perfluorobutyl, or difluoromethyl group. Methods of making the compounds using a copper reagent are also provided.

### FIELD OF THE INVENTION

The invention relates generally to fluoroalkylation and the chemistry of aromatic, nitrogen-containing heterocyclic compounds, in particular pyridines, pyrimidines, and pyrazines.

### DESCRIPTION OF THE RELATED ART

Fluoroalkyl substitution is increasingly used to modulate the activity of biological compounds. The trifluoromethyl substituent is the most common example, found regularly in compounds for pharmaceutical and agricultural applications. Synthetic methodologies are becoming available for introduction of the trifluoromethyl group. One route to trifluoromethyl compounds is the use of chemical intermediates that already contain the trifluoromethyl group, such as α,α,α-trifluorotoluene (also known as trifluoromethylbenzene and benzotrifluoride). This intermediate can be produced from toluene by chlorination of toluene to α,α,α-trichlorotoluene (benzotrichloride) and then substitution of fluorine for chlorine by a displacement reaction with hydrogen fluoride, as reviewed in D. P. Curran et al., Top. Curr. Chem., 1999, 206, 79-105. Once supplied with benzotrifluoride or a related trifluoromethyl aryl building block, additional substitution can be made using standard organic synthetic chemistry practices known to a chemist skilled in the art. Other methods of introducing trifluoromethyl groups have also been described and reviewed. See, for example, Furaya, Kamlet and Ritter in Nature, volume 473, pp. 470-477 (2011); Shibata, Matsnev, and Cahard, Beilstein Journal of Organic Chemistry, volume 6 (2010); and O. A. Tomashenko, et al., “Aromatic Trifluoromethylation with Metal Complexes,” Chem. Rev. 2011, 111, 4475-4521.

There is far less synthetic accessibility to corresponding fluoroalkyl intermediates having fluoroalkyl groups other than trifluoromethyl. Examples of such groups include pentafluoroethyl, heptafluoropropyl, heptafluoroisopropyl, nonafluorobutyl, and difluoromethyl. To access such compounds has required the use of difficult chemistry, and since such chemistry is not generally practised by medicinal chemists skilled in the art, and since the requisite starting materials bearing fluoroalkyl groups other than trifluoromethyl are often commercially unavailable, compounds bearing these alternative fluoroalkyl groups are produced much less frequently, and the utility of such alternative fluoroalkyl groups in medicinal and agricultural chemistry has been often overlooked.

A few reports describe chemistry for introducing fluoroalkyl groups other than trifluoromethyl. By introducing a fluoroalkyl suhstituent from a condensation reaction using a fluoroalkylcarboxylic acid, certain fluoroalkyl substituents can be introduced if the appropriate carboxylic acid is available. For example, Merrell Dow's EP529568 describes pentafluoroethylpeptides derived from pentafluoropropionic acid as elastase inhibitors. Merck's U.S. Pat. No. 3,962,262 reports 1,8-naphthyridine compounds as bronchodilating agents, again derived from condensation reactions on pentafluoropropionic acid. In U.S. Pat. No. 5,092,247, a 3-difluoromethylpyrazole fungicide agent incorporates the difluoromethyl group from ethyl difluoroacetate. However, synthesis starting from a fluoroalkylcarboxylic acid does not usually apply to making fluoroalkylbenzene derivatives. By a different method, U.S. Pat. No. 4,604,406 describes perfluoroalkyl naphthalenes as agents for treating diabetic complications, made by coupling the idonaphthalene with the iodoperfluoroalkane and copper. likewise, Merck's U.S. Pat. No. 5,602,152 patent describes a set of benzoxapines as potassium channel activators, and includes an example with a pentafluoroethyl group borne on a phenyl ring. Among several analogues, pentafluoroethyl-phenyl compounds appear as anti-angiogenesis agents in US2006/194848. There are far fewer reports of fluoroalkyl groups bearing 3 carbons or more. One example, G. D. Searle's U.S. Pat. No. 6,458,803, reports CETP inhibitors for treating atherosclerosis and includes compounds with a pentafluoroethyl or heptafluoropropyl substituent attached to a phenyl ring.

The lack of availability of chemical building blocks bearing fluoroalkyl substituents other than trifluoromethyl, particularly fluoroalkyl substituents having two carbons or more, and especially fluoroalkyl substituents having three carbons or more, has heretofore hindered chemists In exploring the potential utility of compounds bearing such substituents.

### SUMMARY OF THE INVENTION

The present invention provides nitrogen-containing aryl heterocyclic compounds—derivatives of pyridine, pyrimidine, and pyrazine—bearing difluoromethyl and perfluoroalkyl groups larger than trifluoromethyl. These compounds are not known in the prior art, and a general method for their synthesis is heretofore unavailable. Of particular relevance in the practice of this invention is the synthesis of nitrogen-containing aryl heterocyclic compounds bearing perfluoroalkyl groups containing three or more carbons, as the availability of such compounds is extremely limited, and this invention provides the synthesis of many such compounds for the first time.

Pyridine, pyrimidine, and pyrazine derivatives bearing perfluoroalkyl groups larger than trifluroromethyl are prepared by the reaction of a nitrogen-containing heterocyclic aryl iodide or bromide and a copper fluoroalkyl reagent (Complex 1), prepared by a modification of the synthetic approach described in H. Morimoto, et al., “A Broadly Applicable Copper Reagent for Trifluoromethylations and Perfluoroalkylations of Aryl Iodides and Bromides,” Angew. Chem. Int. Ed., 2011, 50, 3793-98, hereby incorporated by reference. Pyridine, pyrimidine, and pyrazine derivatives bearing a difluoromethyl group are prepared by the reaction of a nitrogen-containing heterocyclic aryl iodide or bromide and a difluoromethylating reagent prepared in situ using CuI, CsF, and trimethylsilyldifluoromethane (TMSCF₂H) and the protocol described in Fier and Hartwig, J. Am. Chem. Soc. 2012, 134, 5524-5527, hereby incorporated by reference.

Complex 1

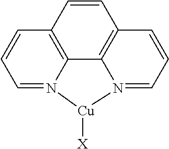

X=CF₂CF₃, CF₂CF₂CF₃, CF(CF₃)₂, CF₂CF₂CF₂CF₃

### DETAILED DESCRIPTION

Fluoroalkyl-containing, nitrogen, aryl heterocyclic compounds are synthesized from the corresponding iodo- or bromo-substituted compound by reaction with a copper fluoroalkyl reagent (Complex 1) or a difluoromethylating reagent prepared in situ using CuI, CsF, and TMSCF₂H. The overall reaction can be described as:

Pyridine Derivatives: Z-pyr-A+Complex 1 or CuI/CsF/TMSCF₂H→Z-pyr-R_{F}

Pyrimidine Derivatives: Z-pyrm-A+Complex 1 or CuI/CsF/TMSCF₂H→Z-pyrm-R_{F}

Pyrazine Derivatives: Z-pyrz-A+Complex 1 or CuI/CsF/TMSCF₂H→Z-pyrz-R_{F}

where “Z-pyr-A” is a pyridine (pyr) heterocycle bearing an iodo or bromo substituent A and another functional group Z, “Z-pyrm-A” is a pyrimidine (pyrm) heterocycle bearing an iodo or bromo substituent A and another functional group Z, and “Z-pyrz-A” is a pyrazine (pyrz) heterocycle bearing an iodo or bromo substituent A and another functional group Z. If the starting heterocycle bears three substituents, A, Z, and Z′ (where A is iodo or bromo), the reaction scheme can be denoted as:

Pyridine Derivatives: Z, Z′-pyr-A+Complex 1 or CuI/CsF/TMSCF₂H→Z,Z′-pyr-R_{F}

Pyrimidine Derivatives: Z,Z′-pyrm-A+Complex 1 or CuI/CsF/TMSCF₂H→Z,Z′-pyrm-R_{F}

Pyrazine Derivatives: Z,Z′-pyrz-A+Complex 1 or CuI/CsF/TMSCF₂H→Z,Z′-pyrz-R_{F}.

Nonlimiting examples of Z and Z′ include chloro, bromo, cyano (CN), methoxy (OCH₃), ethoxy (OCH₂CH₃), benzyloxy (OBz), carbomethoxy (COOCH₃), carboethoxy (COOCH₂CH₃), amide (COHH₂ and NHCOPh), aldehyde (CHO), acetyl (COCH₃), other ketones C(═O)R (where R is lower alkyl or aryl), nitro (NO₂), and protected groups, such as NH—BOC- (where BOC is t-butoxycarbonyl), Bpin (a pinacol boronate ester), and boron-N-methyl-iminodiacetic acid complex (B-MIDA). Protecting groups such a BOC and Bpin can be removed by treatment with acid to yield an amine. The B-MIDA group can be removed at room temperature under mild aqueous conditions using either 1 M NaOH or NaHCO3.

The preparation of the trifluoromethyl homolog to Complex 1 is described in H. Morimoto, et al, “A Broadly Applicable Copper Reagent for Trifluoromethylations and Perfluoroalkylations of Aryl Iodides and Bromides;” Angew. Chem. Int. Ed., 2011, 50, 3793-98. Commercially-available (trimethylsilyl)trifluoromethane, CAS number 81290-20-2, also known as Ruppert-Prakash reagent, is reacted with copper (I) t-butoxide and phenanthroline to produce a stable homolog to Complex 1 in which X=CF₃. Using a modification of the method of Morimoto, et al., reagents hearing other perfluoroalkyl groups—perfluoroethyl, perfluoropropyl, perfluoroisopropyl, and perfluorobutyl—can be prepared. The modified procedure uses a (trimethylsilyl)perfluoroalkane compound reacted with copper t-butoxide coordinated to phenanthroline, which has been previously generated using the reaction of copper mesityl and anhydrous t-butanol in dioxane followed by the addition of phenanthroline under anoxic and anhydrous conditions.

In a further modification of the procedure of Morimoto et al, bromo-substituted nitrogen heterocycles are converted directly into the corresponding fluoroalkyl derivatives, even in cases where there is no other electron-withdrawing group on the aryl ring, by carrying out the reaction in dimethylformamide at a temperature of from about 50° C. to about 110° C., more preferably at a temperature of from about 70° C. to about 100° C., If electron-withdrawing substituents such as nitro, cyano, carbomethoxy, and the like are present on the nitrogen-containing heterocyclic aryl ring, a lower temperature range may be employed, even down to the range of from about 25° C. to about 50° C.

Beneficially, the invention provides the chemical synthesis of new, fluoroalkyl building blocks having utility in medicinal chemistry, agricultural chemistry, and other applications by virtue of the larger, previously unavailable fluoroalkyl side chain. In one embodiment of the invention, a nitrogen-containing heterocyclic iodoarene or bromoarene is converted into the corresponding perfluoroalkylarene using a perfluoroalkyl copper reagent prepared as described above containing a perfluoroalkyl group, X. For example, 2-bromo-6-carbomethoxypyridine is converted to heretofore unknown 2-pentafluoroethyl-6-carbomemoxypyridine, 2-heptafluoropropyl-6-carbomethoxypyridine, 2-heptafluoroisopropyl-6-carbomethxypyridine, or 2-nonafluorobutyl-6-carbomethoxypyridine by reaction with pentafluoroethyl( 1,10-phenanthroline)copper, heptafluoropropyl(1,10-phenanthroline)copper, heptafluoroisopropyl(1,10-phenanthroline)copper, or nonafluorobutyl(1,10-phenanthroline)copper, respectively. In another embodiment, 2-bromo-6-cyanopyridine or 2-bromo-6-carbomethoxypyridine is convened to the heretofore unknown 2-difluoromethyl-6-cyanopyridine or 2-difluoromethyl-6-carbomethoxypyridine, respectively, by reaction with copper iodide, cesium fluoride, and trimethyl(difluoromethyl)silane. In a further embodiment, 3-bromo-6-carbomethoxypyridine is converted to heretofore unknown 3-pentafluoroethyl-6-carbomethoxypyridine, 3-heptafluoropropyl-6-carbomethoxypyridine, 3-heptafluoroisopropyl-6-carbomethoxypyridine, or 3-nonafluorobutyl-6-carbomethoxypyridine by reaction with pentafluoroethyl(1,10-phenanthroline)copper, heptafluoropropyl(1,10-phenanthroline)copper, heptafluoroisopropyl(1,10-phenanthroline)copper, or nonafluorobutyl(1,10-phenanthroline)copper, respectively. In a further embodiment, 3-bromo-6-cyanopyridine or 3-bromo-6-carbomethoxypyridine is converted to the heretofore unknown 3-difluoromethyl-6-cyanopyridine or 3difluoromethyl-6-carbomethoxypyridine, respectively, by reaction with copper iodide, cesium fluoride, and trimethyl(difluoromethyl)silane. In a further embodiment of this invention, 4-bromo-6 carbomethoxypyridine is converted to heretofore unknown 4-pentafluoroethyl-6-carbomethoxypyxidine, 4-heptafluoropropyl-6-carbomethoxypyodine, 4-heptafluoroisopropyl-carbomethoxypyridine, or 4-nonafluorobutyl-6-carbomethoxypyridine by reaction with pentafluoroethyl(1,10-phenanthroline)copper, heptafluoropropyl(1,10-phenathroline)copper, heptafluoroisopropyl(1,10-phenanthroline)copper, or nonafluorobutyl(1,10-phenanthroline)copper, respectively. In a further embodiment, 4-bromo-6-cyanopyridine or 4-bromo-6-carbomethoxypyridine is converted to the heretofore unknown 4-difluoromethyl-6-cyanopyridine or 4-difluoromethyl-6-carbomethoxypyridine, respectively, by reaction with copper iodide, cesium fluoride, and trimethyl(difluoromethyl)silane. In a further embodiment, 5-bromo-6-carbomethoxypyridine is converted to heretofore unknown 5-pentafluoroethyl-6-carbomethoxypyridine, 5-heptafluoropropyl-6-carbomethoxypyridine, 5-heptafluoroisopropyl-6-carbomethoxypyridine, or 5-nonaafluorobutyl-6-carbomethoxypyridine by reaction with pentafluoroethyl(1,10-phenanthroline)copper, heptafluoropropyl(1,10-phenanthroline)copper, heptafluoroisopropyl( 1,10-phenanthroline)copper, or nonafluorobutyl(1,10-phenanthroline)copper, respectively. In a further embodiment, 5-bromo-6cyanopyridine or 5-bromo-6-carbomethoxypyridine is converted to the heretofore unknown 5-difluoromethyl-6-cyanopyridine or 3-difluoromethyl-6-carbomethoxypyridine, respectively, by reaction with copper iodide, cesium fluoride, and trimethyl(difluoromethyl)silane.

In all of the above embodiments, a carbomethoxy or cyano group can be replaced by an alternative functional chemical functional group of utility to chemists, such as —CHO (aldehyde), —C(═O)R (ketone, in which R is lower alkyl or aryl), NO₂ (nitro), —NH-BOC, where BOC is t-butyloxycarbonyl), Bpin, where Bpin represents the pinacol boronate ester), and other groups. The protecting group, BOC, can be removed with. acid, to yield an amine (NH₂).

Fluoroalkylating Reagents

Pentafluoroethyl(1,10-phenanthroline)copper (Complex 1, where X=CF₂CF₃) is prepared as follows, Anhydrous CuCl (1.1 gram , 1 mmol, 1.1 eq) of anhydrous CuCl is suspended in 10 mL of THF at −30° C. and 10.0 mL of MesMgBr (1.0 M in THF, 10.0 mmoL, 1.0 eq) is added slowly. The solution is allowed to warm to room temperature and stirred for 3 hours. Six mL of anhydrous dioxane is added to precipitate the magnesium salts and the solid is separated away by filtration or cannula transfer. To the light green solution is added 950 μL tBuOH (1.1 mmol, 1.1 eq). The light yellow solution is stirred for 1 hour. Then, 1.785 g (10.0 mmol, 1.0 eq) of 1,10-phenanthroline in 10 mL of THF is added at once to the solution of CuOtBu to give a homogenous dark-purple solution. After 30 minutes, 2 mL (11 mmol, 1.1 eq) of (trimethylsilyl)pentafluoroethane is added neat and stirred at room temperature overnight, (phen)CuCF₂CF₃ precipitates as a light-brown solid and is collected on a glass-frit and washed with diethyl ether until the filtrate is colorless. The product is dried in vacuo and stored under nitrogen or argon. The perfluoropropyl perfluoroisopropyl, and perfluorobutyl reagents can be prepared in analogous fashion, replacing (trimethylsilyl)pentafluoroethane with (trimethylsilyl)heptafluoropropane, (trimethylsilyl)heptafluoroisopropane, and (trimemylsilyl)nonafluorobutane, respectively. Some perfluoroalkyltrimethylsilanes are commercially available, for example, trifluoromethyltrimethylsilane “TMSCF₃” (also known as “Ruppert's reagent” and “Ruppert-Prakash reagent”) and perfluoroethyltrimethylsilane (“TMSCF₂CF₃”). Others are prepared in a manner analogous to the preparation of Ruppert's reagent, namely reaction of trimethylsilyl chloride with the appropriate perfluorobromide (CF₃CF₂Br, CF₃CF₂CF₂Br, CF₃CF₂CF₃CF₂Br, CF₃CF₂CF₂CF₂CF₂Br. Electrochemical methods for the preparation of Ruppert's reagent and its higher congeners have also been reported, for example Aymard et al in Tetrahedron Letters, 46, 8623-8624 (1994), hereby incorporated by reference.

Difluoromethyl compounds according to this invention are prepared using a different protocol, according to Fier and Hartwig, J. Am. Chem. Soc. 2012, 134, 5524-5527. In a nitrogen-filled glove box or under a strict nitrogen or argon atmosphere, aryl iodide (10 mmol, 1 equiv) is combined with the mixture of copper iodide (10 mmol, 1.91 grams, 1 equiv), and cesium fluoride (30 mmol, 4.56 grams, 3 equiv) in a 200 mL reaction vial. To this vial is added 50 mL of anhydrous N-methylpyrollidone, followed by trimethl(difluoromethyl)silane (50 mmol, 5 equiv). The reaction mixture is heated in a sealed vessel at 120° C. for 24 hours. The pressure increases during the reaction due to the formation of volatile fluorotrimethylsilane (Me₃SiF) as a stoichiometric product. The resulting dark red solution is then cooled to room temperature and diluted with 200 mL of diethyl ether. The resulting mixture is filtered over Celite, washed with an additional 200 mL of diethyl ether, and transferred to a separatory funnel The mixture is then washed with 5×100 mL of H₂O and 1×100 mL of brine, dried over anhydrous MgSO₄, filtered, and concentrated under vacuum. The crude product can be purified by column chromatography on silica gel with pentane or pentane/ether mixtures as the eluent.

Synthesis of Fluoroalkyl-Substituted Pyridines, Pyrimidines, and Pyrazines.

Iodo- and bromo-substituted pyridine, pyrmidine, and pyrazine compounds, bearing one or more additional functional groups (Z, Z′) are commercially available from a number of vendors, such as Sigma-Aldrich. In Procedures A-E, general synthetic methodologies are provided for fluoroalkylating the starting compounds to provide fluoroalkyl-substituted pyridines, pyrimidines, and pyrazines. For convenience, the starting heterocyclic compound is generically referred to as an “aryl iodide or bromide.” Although each procedure explicitly refers to the aryl iodide only, it will be understood that a bromo analog can be used in the alternative.

Procedure A—General Method for Synthesis of Pentafluoroethyl-Substituted, Nitrogen-Containing Heterocyclic Compounds from the Corresponding Aryl Iodide or Bromide.

To a 20 mL vial equipped with a stir bar is added aryl iodide 3 (0.50 mmol), 1,10-phenanthroline pentafluoroethyl copper (272 mg, 0.75 mmol, 1.5 equiv), and dimethyl formamide (2.0 mL). The mixture is stirred at a temperature of 25 to 50° C. for 16 to 18 hours. After this time, stirring is stopped, and the reaction mixture is diluted with 10 mL of diethyl ether and filtered through a pad of Celite. The Celite pad is washed with an additional 20 mL of diethyl ether, and the combined filtrate is transferred to a separatory funnel, and washed with 1 Molar aqueous HCl, saturated aqueous NaHCO₃ solution, and saturated aqueous NaCl, and then dried over anhydrous Na₂SO4. After filtration and evaporation of the solvent, the crude mixture is purified by flash silica gel column chromatography using pentane/diethyl ether or pentane as eluent to give the pentafluoroethyl-substituted aryl product

Procedure B—General Method for Synthesis of Heptafluoropropyl-Substituted, Nitrogen-Containing Heterocyclic Compounds from the Corresponding Aryl Iodide or Bromide.

To a 20 mL vial equipped with a stir bar is added aryl iodide 3 (0.50 mmol), 1,10-phenanthroline heptafluoropropyl copper (309 mg, 0.75 mmol, 1.5 equiv), and dimethyl formamide (2.0 mL). The mixture is stirred at a temperature of 25 to 50° C. for 16 to 18 hours. After this time, stirring is stopped, and the reaction mixture is diluted with 10 mL of diethyl ether and filtered through a pad of Celite. The Celite pad is washed with an additional 20 mL of diethyl ether, and the combined filtrate is transferred to a separatory funnel and washed with 1 Molar aqueous HCl, saturated aqueous NaHCO₃ solution, and saturated aqueous NaCl, and then dried over anhydrous Na₂SO4. After filtration and evaporation of the solvent, the crude mixture is purified by flash silica gel column chromatography using pentane/diethyl ether or pentane as eluent to give the heptafluoropropyl-substituted aryl product

Procedure C—General Method for Synthesis of Perfluorobutyl-Substituted, Nitrogen-Containing Heterocyclic Compounds from the Corresponding Aryl Iodide or Bromide.

To a 20 mL vial equipped with a stir bar is added aryl iodide 3 (0.50 mmol), 1,10-phenanthroline nonafluorobutyl copper (347 mg, 0.75 mmol, 1.5 equiv), and dimethyl formamide (2.0 mL). The mixture is stirred at a temperature of 25 to 50° C. for 16 to 18 hours. After this time, stirring is stopped, and the reaction mixture is diluted with 10 mL of diethyl ether and filtered through a pad of Celite. The Celite pad is washed with an additional 20 mL of diethyl ether, and the combined filtrate is transferred to a separatory funnel and washed with 1 Molar aqueous HCl. saturated aqueous NaHCO₃ solution, and saturated aqueous NaCl, and then dried over anhydrous Na₂SO4. After filtration and evaporation of the solvent, the crude mixture is purified by flash silica gel column chromatography using pentane/diethyl ether or pentane as eluent to give the nonafluorobutyl-substituted aryl product.

Procedure D—General Method for Synthesis of Difluoromethyl-Substituted Nitrogen Heterocyclic Compounds from the Corresponding Iodide or Bromide.

In a nitrogen-filled glove box, the nitrogen-containing heterocyclic iodide or bromide (0.5 mmol, 1 equiv), copper iodide (0.5 mmol, 1 eq), and cesium fluoride 0.5 mmol, 1 equiv) are combined in a 20 mL vial. To this vial is added 2.5 mL of anhydrous N-methypyrolidine, followed by trimethyl(difluoromethyl)silane (2.5 mmol, 5 equiv). The reaction mixture is heated in a sealed vessel at 120° C. for 24 h. Note: the pressure increases during the reaction due to the formation of volatile fluorotrimethylsilane (Me₃SiF) as a stoichiometric byproduct. The dark red solution is then cooled to room temperature, and diluted with 15 mL of diethyl ether. The mixture is filtered over Celite, washed with an additional 20 mL of Et2 O, and transferred to a separatory funnel. The mixture is washed with 5×20 mL of H₂O and 1×20 mL of saturated aqueous NaCl, dried over anhydrous MgSO₄, filtered, and concentrated under vacuum. The crude product is purified by column chromatography on silica gel with pentane or a pentane/diethyl ether mixture as the eluent.

Procedure E—General Method for Synthesis of Heptafluoroisopropyl-Substituted, Nitrogen-Containing Heterocyclic Compounds from the Corresponding Aryl Iodide or Bromide.

To a 20 mL vial equipped with a stir bar is added aryl iodide 3 (0.50 mmol), 1,10-phenanthroline heptafluoroisopropyl copper (309 mg, 0.75 mmol, 1.5 equiv), and dimethyl formamide (2.0 mL). The mixture is stirred at a temperature of 25 to 50° C. for 16 to 18 hours. After this time, stirring is stopped, and the reaction mixture is diluted with 10 mL of diethyl ether and filtered through a pad of Celite. The Celite pad is washed with an additional 20 mL of diethyl ether, and the combined filtrate is transferred to a separatory funnel and washed with 1 Molar aqueous HCl, saturated aqueous NaHCO₃ solution, and saturated aqueous NaCl, and then dried over anhydrous Na₂SO4. After filtration and evaporation of the solvent, the crude mixture is purified by flash silica gel column chromatography using pentane/diethyl ether or pentane as eluent to give the heptafluoroisopropyl-substituted aryl product.

### EXAMPLES

Using the synthetic protocols described in Procedures A-E above, a number of perfluoroalkyl-substituted pyridines, pyrimidines, and pyrazines are prepared from the corresponding aryl iodide or aryl bromide precursors. In the examples tabulated below, a heterocyclic compound based on pyridine, pyrimidine, or pyrazine is presented, with two or more substituents, A Z, and Z′ attached thereto. The substituents are identified for both the starting compound and the product, and the fluoroalkyl group that is introduced is also identified. The following abbreviations are used: Bz=benzyl, BOC=benzyloxycarbonyl, Bpin=pinacol boronate, BMIDA=Boron-N-methyl-iminodiacetic acid complex.

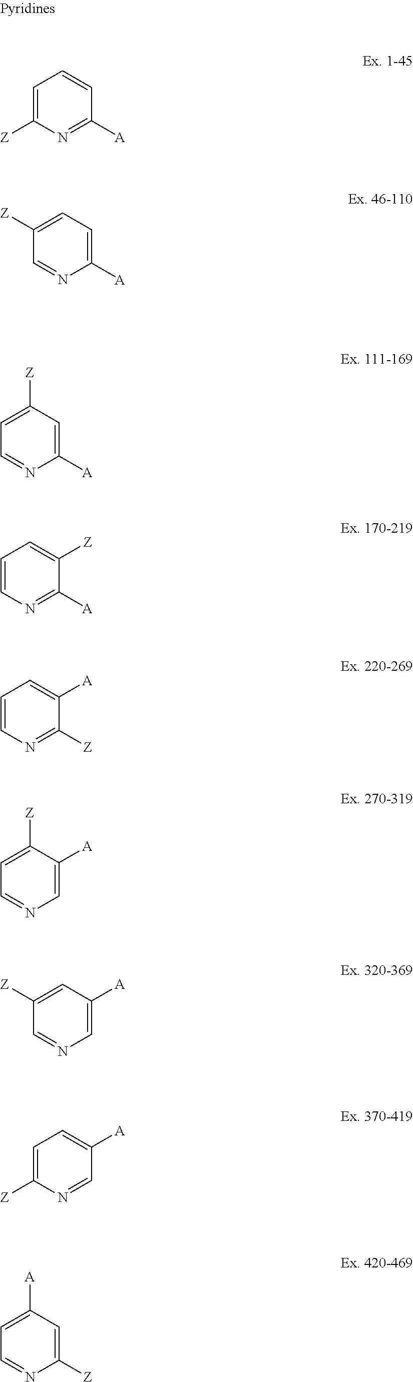

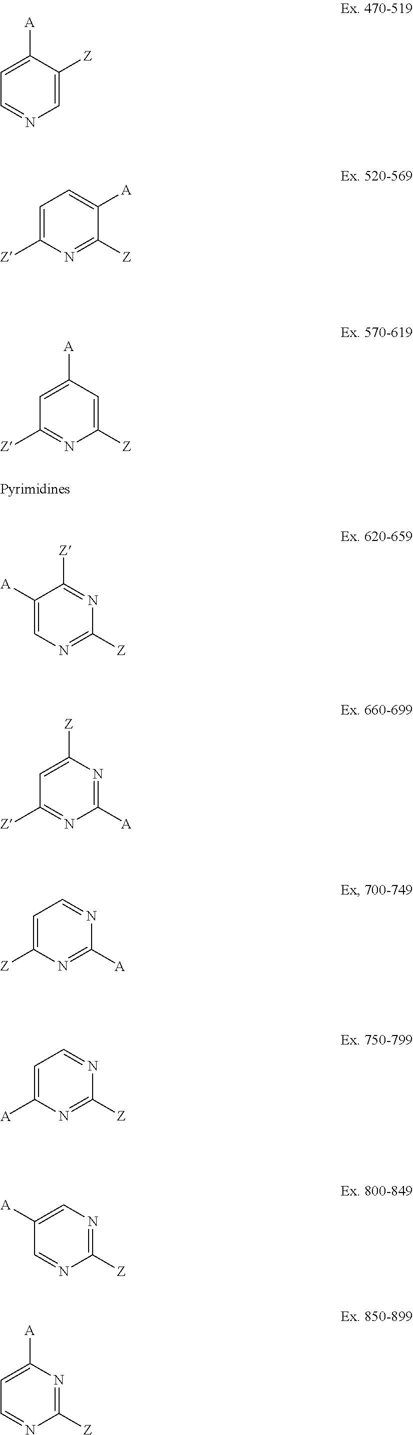

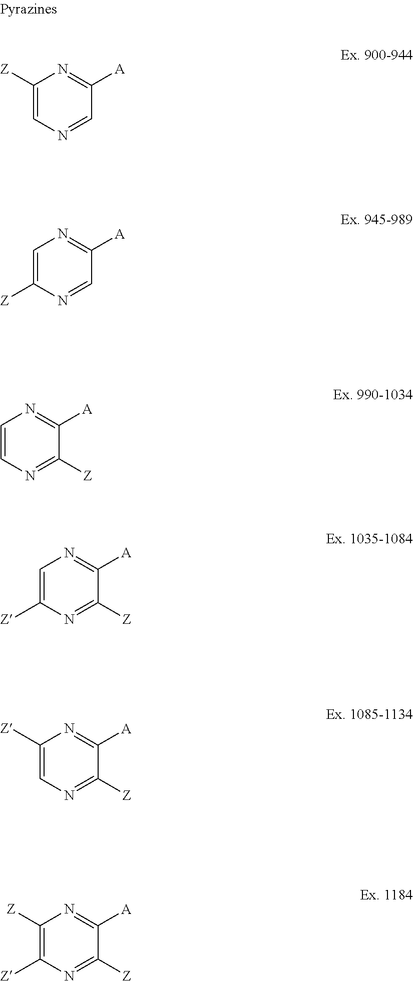

### Examples 1-9: 2-Pentafluoroethyl-6-Substituted Pyridines

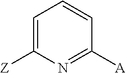

Procedure A is used to prepare the pentafluoroethyl derivative from the corresponding iodide or bromide.

| Example | Starting material | Product |
|---|---|---|
| 1 | A = Br; Z = Cl | A = CF₂CF₃; Z = Cl |
| 2 | A = I; Z = Br | A = CF₂CF₃; Z = Br |
| 3 | A = I; Z = CO₂C₂H₅ | A = CF₂CF₃; Z = CO₂C₂H₂ |
| 4 | A = I; Z = CONH₂ | A = CF₂CF₃; Z = CONH₂ |
| 5 | A = I; Z = COCH₃ | A = CF₂CF₃; Z = COCH₃ |
| 6 | A = I; Z = CHO | A = CF₂CF₃; Z = CHO |
| 7 | A = I; Z = OBz | A = CF₂CF₃; Z = OBz |
| 8 | A = I; Z = NH—BOC | A = CF₂CF₃; Z = NH—BOC |
| 9 | A = Br; Z = CN | A = CF₂CF₃; Z = CN |

### Examples 10-18: 2-Heptafluoropropyl-6-Substituted Pyridines

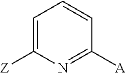

Procedure B is used to prepare the heptafluoropropyl derivative from the corresponding iodide or bromide.

| Example | Starting material | Product |
|---|---|---|
| 10 | A = Br; Z = Cl | A = CF₂CF₂CF₃; Z = Cl |
| 11 | A = I; Z = Br | A = CF₂CF₂CF₃; Z = Br |
| 12 | A = I; Z = CO₂C₂H₅ | A = CF₂CF₂CF₃; Z = CO₂C₂H₂ |
| 13 | A = I; Z = CONH₂ | A = CF₂CF₂CF₃; Z = CONH₂ |
| 14 | A = I; Z = COCH₃ | A = CF₂CF₂CF₃; Z = COCH₃ |
| 15 | A = I; Z = CHO | A = CF₂CF₂CF₃; Z = CHO |
| 16 | A = I; Z = OBz | A = CF₂CF₂CF₃; Z = OBz |
| 17 | A = I; Z = NH—BOC | A = CF₂CF₂CF₃; Z = NH—BOC |
| 18 | A = Br; Z = CN | A = CF₂CF₂CF₃; Z = CN |

### Examples 19-27: 2-Nonafluorobutyl-6-Substituted Pyridines

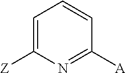

Procedure C is used to prepare the nonafluorobutyl derivative from the corresponding iodide or bromide.

| Example | Starting material | Product |
|---|---|---|
| 19 | A = Br; Z = Cl | A = CF₂CF₂CF₂CF₃; Z = Cl |
| 20 | A = I; Z = Br | A = CF₂CF₂CF₂CF₃; Z = Br |
| 21 | A = I; Z = CO₂C₂H₅ | A = CF₂CF₂CF₂CF₃; Z = CO₂C₂H₂ |
| 22 | A = I; Z = CONH₂ | A = CF₂CF₂CF₂CF₃; Z = CONH₂ |
| 23 | A = I; Z = COCH₃ | A = CF₂CF₂CF₂CF₃; Z = COCH₃ |
| 24 | A = I; Z = CHO | A = CF₂CF₂CF₂CF₃; Z = CHO |
| 25 | A = I; Z = OBz | A = CF₂CF₂CF₂CF₃; Z = OBz |
| 26 | A = I; Z = NH—BOC | A = CF₂CF₂CF₂CF₃; Z = NH—BOC |
| 27 | A = Br; Z = CN | A = CF₂CF₂CF₂CF₃; Z = CN |

### Examples 28-36: 2-Difluoromethyl-6-Substituted Pyridines

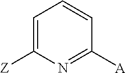

Procedure D is used to prepare the difluoromethyl derivative from the corresponding iodide or bromide.

| Example | Starting material | Product |
|---|---|---|
| 28 | A = Br; Z = Cl | A = CHF₂; Z = Cl |
| 29 | A = I; Z = Br | A = CHF₂; Z = Br |
| 30 | A = I; Z = CO₂C₂H₅ | A = CHF₂; Z = CO₂C₂H₂ |
| 31 | A = I; Z = CONH₂ | A = CHF₂; Z = CONH₂ |
| 32 | A = I; Z = COCH₃ | A = CHF₂; Z = COCH₃ |
| 33 | A = I; Z = CHO | A = CHF₂; Z = CHO |
| 34 | A = I; Z = OBz | A = CHF₂; Z = OBz |
| 35 | A = I; Z = NH—BOC | A = CHF₂; Z = NH—BOC |
| 36 | A = Br; Z = CN | A = CHF₂; Z = CN |

### Examples 37-45: 2-Heptafluoroisopropyl-6-Substituted Pyridines

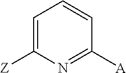

Procedure E is used to prepare the heptafluoroisopropyl derivative from the corresponding iodide or bromide.

| Example | Starting material | Product |
|---|---|---|
| 37 | A = Br; Z = Cl | A = CF(CF₃)₂; Z = Cl |
| 38 | A = I; Z = Br | A = CF(CF₃)₂; Z = Br |
| 39 | A = I; Z = CO₂C₂H₅ | A = CF(CF₃)₂; Z = CO₂C₂H₂ |
| 40 | A = I; Z = CONH₂ | A = CF(CF₃)₂; Z = CONH₂ |
| 41 | A = I; Z = COCH₃ | A = CF(CF₃)₂; Z = COCH₃ |
| 42 | A = I; Z = CHO | A = CF(CF₃)₂; Z = CHO |
| 43 | A = I; Z = OBz | A = CF(CF₃)₂; Z = OBz |
| 44 | A = I; Z = NH—BOC | A = CF(CF₃)₂; Z = NH—BOC |
| 45 | A = Br; Z = CN | A = CF(CF₃)₂; Z = CN |

### Examples 46-58: 2-Pentafluoroethyl-5-Substituted Pyridines

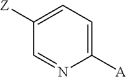

Procedure A is used to prepare the pentafluoroethyl derivative from the corresponding iodide or bromide.

| Example | Starting material | Product |
|---|---|---|
| 46 | A = Br; Z = Cl | A = CF₂CF₃; Z = Cl |
| 47 | A = Br; Z = CO₂C₂H₅ | A = CF₂CF₃; Z = CO₂C₂H₅ |
| 48 | A = Br; Z = CONH₂ | A = CF₂CF₃; Z = CONH₂ |
| 49 | A = Br; Z = COCH₃ | A = CF₂CF₃; Z = COCH₃ |
| 50 | A = Br; Z = CHO | A = CF₂CF₃; Z = CHO |
| 51 | A = Br; Z = OBz | A = CF₂CF₃; Z = OBz |
| 52 | A = Br; Z = Br | A = CF₂CF₃; Z = Br |
| 53 | A = I; Z = Br | A = CF₂CF₃; Z = Br |
| 54 | A = Br; Z = CN | A = CF₂CF₃; Z = CN |
| 55 | A = Br; Z = Bpin | A = CF₂CF₃; Z = Bpin |
| 56 | A = Br; Z = BMIDA | A = CF₂CF₃; Z = BMIDA |
| 57 | A = Br; Z = NO₂ | A = CF₂CF₃; Z = NO₂ |
| 58 | A = I; Z = NH—BOC | A = CF₂CF₃; Z = NH—BOC |

### Examples 59-71: 2-Heptafluoropropyl-5-Substituted Pyridines

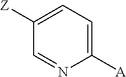

Procedure B is used to prepare the heptafluoropropyl derivative from the corresponding iodide or bromide.

| Example | Starting material | Product |
|---|---|---|
| 59 | A = Br; Z = Cl | A = CF₂CF₂CF₃; Z = Cl |
| 60 | A = Br; Z = CO₂C₂H₅ | A = CF₂CF₂CF₃; Z = CO₂C₂H₅ |
| 61 | A = Br; Z = CONH₂ | A = CF₂CF₂CF₃; Z = CONH₂ |
| 62 | A = Br; Z = COCH₃ | A = CF₂CF₂CF₃; Z = COCH₃ |
| 63 | A = Br; Z = CHO | A = CF₂CF₂CF₃; Z = CHO |
| 64 | A = I; Z = OBz | A = CF₂CF₂CF₃; Z = OBz |
| 65 | A = Br; Z = Br | A = CF₂CF₂CF₃; Z = Br |
| 66 | A = I; Z = Br | A = CF₂CF₂CF₃; Z = Br |
| 67 | A = Br; Z = CN | A = CF₂CF₂CF₃; Z = CN |
| 68 | A = Br; Z = Bpin | A = CF₂CF₂CF₃; Z = Bpin |
| 69 | A = Br; Z = BMIDA | A = CF₂CF₂CF₃; Z = BMIDA |
| 70 | A = Br; Z = NO₂ | A = CF₂CF₂CF₃; Z = NO₂ |
| 71 | A = I; Z = NH—BOC | A = CF₂CF₂CF₃; Z = NH—BOC |

### Examples 72-84: 2-Nonafluorobutyl-5-Substituted Pyridines

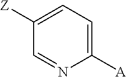

Procedure C is used to prepare the nonafluorobutyl derivative from the corresponding iodide or bromide.

| Example | Starting Material | Product |
|---|---|---|
| 72 | A = Br; Z = Cl | A = CF₂CF₂CF₂CF₃; Z = Cl |
| 73 | A = Br; Z = CO₂C₂H₅ | A = CF₂CF₂CF₂CF₃; Z = CO₂C₂H₅ |
| 74 | A = Br; Z = CONH₂ | A = CF₂CF₂CF₂CF₃; Z = CONH₂ |
| 75 | A = Br; Z = COCH₃ | A = CF₂CF₂CF₂CF₃; Z = COCH₃ |
| 76 | A = Br; Z = CHO | A = CF₂CF₂CF₂CF₃; Z = CHO |
| 77 | A = I; Z = OBz | A = CF₂CF₂CF₂CF₃; Z = OBz |
| 78 | A = Br; Z = Br | A = CF₂CF₂CF₂CF₃; Z = Br |
| 79 | A = I; Z = Br | A = CF₂CF₂CF₂CF₃; Z = Br |
| 80 | A = Br; Z = CN | A = CF₂CF₂CF₂CF₃; Z = CN |
| 81 | A = Br; Z = Bpin | A = CF₂CF₂CF₂CF₃; Z = Bpin |
| 82 | A = Br; Z = BMIDA | A = CF₂CF₂CF₂CF₃; Z = BMIDA |
| 83 | A = Br; Z = NO₂ | A = CF₂CF₂CF₂CF₃; Z = NO₂ |
| 84 | A = I; Z = NH—BOC | A = CF₂CF₂CF₂CF₃; Z = NH—BOC |

### Examples 85-97: 2-Difluoromethyl-Substituted Pyridines

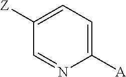

Procedure D is used to prepare the difluoromethyl derivative from the corresponding iodide or bromide.

| Example | Starting material | Product |
|---|---|---|
| 85 | A = Br; Z = Cl | A = CHF₂; Z = Cl |
| 86 | A = Br; Z = CO₂C₂H₅ | A = CHF₂; Z = CO₂C₂H₅ |
| 87 | A = Br; Z = CONH₂ | A = CHF₂; Z = CONH₂ |
| 88 | A = Br; Z = COCH₃ | A = CHF₂; Z = COCH₃ |
| 89 | A = Br; Z = CHO | A = CHF₂; Z = CHO |
| 90 | A = I; Z = OBz | A = CHF₂; Z = OBz |
| 91 | A = Br; Z = Br | A = CHF₂; Z = Br |
| 92 | A = I; Z = Br | A = CHF₂; Z = Br |
| 93 | A = Br; Z = CN | A = CHF₂; Z = CN |
| 94 | A = Br; Z = Bpin | A = CHF₂; Z = Bpin |
| 95 | A = Br; Z = BMIDA | A = CHF₂; Z = BMIDA |
| 96 | A = Br; Z = NO₂ | A = CHF₂; Z = NO₂ |
| 97 | A = I; Z = NH—BOC | A = CHF₂; Z = NH—BOC |

### Examples 98-110: 2-Heptafluoropropyl-5-Substituted Pyridines

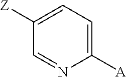

Procedure E is used to prepare the heptafluoropropyl derivative from the corresponding iodide or bromide.

| Example | Starting Material | Product |
|---|---|---|
| 98 | A = Br; Z = Cl | A = CF(CF₃)₂; Z = Cl |
| 99 | A = Br; Z = CO₂C₂H₅ | A = CF(CF₃)₂; Z = CO₂C₂H₅ |
| 100 | A = Br; Z = CONH₂ | A = CF(CF₃)₂; Z = CONH₂ |
| 101 | A = Br; Z = COCH₃ | A = CF(CF₃)₂; Z = COCH₃ |
| 102 | A = Br; Z = CHO | A = CF(CF₃)₂; Z = CHO |
| 103 | A = I; Z = OBz | A = CF(CF₃)₂; Z = OBz |
| 104 | A = Br; Z = Br | A = CF(CF₃)₂; Z = Br |
| 105 | A = I; Z = Br | A = CF(CF₃)₂; Z = Br |
| 106 | A = Br; Z = CN | A = CF(CF₃)₂; Z = CN |
| 107 | A = Br; Z = Bpin | A = CF(CF₃)₂; Z = Bpin |
| 108 | A = Br; Z = BMIDA | A = CF(CF₃)₂; Z = BMIDA |
| 109 | A = Br; Z = NO₂ | A = CF(CF₃)₂; Z = NO₂ |
| 110 | A = I; Z = NH—BOC | A = CF(CF₃)₂; Z = NH—BOC |

### Examples 111-123: 2-Pentafluoroethyl-4-Substituted Pyridines

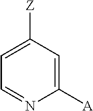

Procedure A is used to prepare the pentafluoroethyl derivative from the corresponding iodide or bromide.

| Example | Starting material | Product |
|---|---|---|
| 111 | A = Br; Z = Cl | A = CF₂CF₃; Z = Cl |
| 112 | A = Br; Z = CO₂C₂H₅ | A = CF₂CF₃; Z = CO₂C₂H₅ |
| 113 | A = Br; Z = CONH₂ | A = CF₂CF₃; Z = CONH₂ |
| 114 | A = Br; Z = COCH₃ | A = CF₂CF₃; Z = COCH₃ |
| 115 | A = Br; Z = CHO | A = CF₂CF₃; Z = CHO |
| 116 | A = I; Z = OBz | A = CF₂CF₃; Z = OBz |
| 117 | A = Br; Z = Br | A = CF₂CF₃; Z = Br |
| 118 | A = I; Z = Br | A = CF₂CF₃; Z = Br |
| 119 | A = Br; Z = CN | A = CF₂CF₃; Z = CN |
| 120 | A = Br; Z = Bpin | A = CF₂CF₃; Z = Bpin |
| 121 | A = Br; Z = BMIDA | A = CF₂CF₃; Z = BMIDA |
| 122 | A = Br; Z = NO₂ | A = CF₂CF₃; Z = NO₂ |
| 123 | A = I; Z = NH—BOC | A = CF₂CF₃; Z = NH—BOC |

### Examples 124-136: 2-Heptafluoropropyl-4-Substituted Pyridines

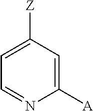

Procedure B is used to prepare the heptafluoropropyl derivative from, the corresponding iodide or bromide.

| Example | Starting material | Product |
|---|---|---|
| 124 | A = Br; Z = Cl | A = CF₂CF₂CF₃; Z = Cl |
| 125 | A = Br; Z = CO₂C₂H₅ | A = CF₂CF₂CF₃; Z = CO₂C₂H₅ |
| 126 | A = Br; Z = CONH₂ | A = CF₂CF₂CF₃; Z = CONH₂ |
| 127 | A = Br; Z = COCH₃ | A = CF₂CF₂CF₃; Z = COCH₃ |
| 128 | A = Br; Z = CHO | A = CF₂CF₂CF₃; Z = CHO |
| 129 | A = I; Z = OBz | A = CF₂CF₂CF₃; Z = OBz |
| 130 | A = Br; Z = Br | A = CF₂CF₂CF₃; Z = Br |
| 131 | A = I; Z = Br | A = CF₂CF₂CF₃; Z = Br |
| 132 | A = Br; Z = CN | A = CF₂CF₂CF₃; Z = CN |
| 133 | A = Br; Z = Bpin | A = CF₂CF₂CF₃; Z = Bpin |
| 144 | A = Br; Z = BMIDA | A = CF₂CF₂CF₃; Z = BMIDA |
| 135 | A = Br; Z = NO₂ | A = CF₂CF₂CF₃; Z = NO₂ |
| 136 | A = I; Z = NH—BOC | A = CF₂CF₂CF₃; Z = NH—BOC |

### Examples 137-149: 2-Nonafluorobutyl-4-Substituted Pyridines

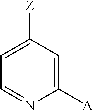

Procedure C is used to prepare the nonafluorobutyl derivative from the corresponding iodide or bromide.

| Example | Starting material | Product |
|---|---|---|
| 137 | A = Br; Z = Cl | A = CF₂CF₂CF₂CF₃; Z = Cl |
| 138 | A = Br; Z = CO₂C₂H₅ | A = CF₂CF₂CF₂CF₃; Z = CO₂C₂H₅ |
| 139 | A = Br; Z = CONH₂ | A = CF₂CF₂CF₂CF₃; Z = CONH₂ |
| 140 | A = Br; Z = COCH₃ | A = CF₂CF₂CF₂CF₃; Z = COCH₃ |
| 141 | A = Br; Z = CHO | A = CF₂CF₂CF₂CF₃; Z = CHO |
| 142 | A = I; Z = OBz | A = CF₂CF₂CF₂CF₃; Z = OBz |
| 143 | A = Br; Z = Br | A = CF₂CF₂CF₂CF₃; Z = Br |
| 144 | A = I; Z = Br | A = CF₂CF₂CF₂CF₃; Z = Br |
| 145 | A = Br; Z = CN | A = CF₂CF₂CF₂CF₃; Z = CN |
| 146 | A = Br; Z = Bpin | A = CF₂CF₂CF₂CF₃; Z = Bpin |
| 147 | A = Br; Z = BMIDA | A = CF₂CF₂CF₂CF₃; Z = BMIDA |
| 148 | A = Br; Z = NO₂ | A = CF₂CF₂CF₂CF₃; Z = NO₂ |
| 149 | A = I; Z = NH—BOC | A = CF₂CF₂CF₂CF₃; Z = NH—BOC |

### Examples 150-159: 2-Difluoromethyl-4-Substituted Pyridines

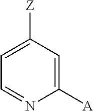

Procedure D is used to prepare the difluoromethyl derivative from the corresponding iodide or bromide.

| Example | Starting material | Product |
|---|---|---|
| 150 | A = I; Z = Cl | A = CHF₂; Z = Cl |
| 151 | A = I; Z = OC₂H₅ | A = CHF₂; Z = OC₂H₅ |
| 152 | A = I; Z = O—Bz | A = CHF₂; Z = O—Bz |
| 153 | A = I; Z = Br | A = CHF₂; Z = Br |
| 154 | A = I; Z = HC═O | A = CHF₂; Z = HC═O |
| 155 | A = I; Z = CO₂CH₃ | A = CHF₂; Z = CO₂CH₃ |
| 156 | A = I; Z = COCH₃ | A = CHF₂; Z = COCH₃ |
| 157 | A = I; Z = CONH₂ | A = CHF₂; Z = CONH₂ |
| 158 | A = I; Z = CN | A = CHF₂; Z = CN |
| 159 | A = I; Z = NH—BOC | A = CHF₂; Z = NH—BOC |

### Examples 160-169: 2-Heptafluoroisopropyl-4-Substituted Pyridines

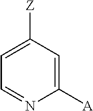

Procedure E is used to prepare the heptafluoroisopropyl derivative from the corresponding iodide.

| Example | Starting material | Product |
|---|---|---|
| 160 | A = I; Z = Cl | A = CF(CF₃)₂; Z = Cl |
| 161 | A = I; Z = OC₂H₅ | A = CF(CF₃)₂; Z = OC₂H₅ |
| 162 | A = I; Z = O—Bz | A = CF(CF₃)₂; Z = O—Bz |
| 163 | A = I; Z = Br | A = CF(CF₃)₂; Z = Br |
| 164 | A = I; Z = HC═O | A = CF(CF₃)₂; Z = HC═O |
| 165 | A = I; Z = CO₂CH₃ | A = CF(CF₃)₂; Z = CO₂CH₃ |
| 166 | A = I; Z = COCH₃ | A = CF(CF₃)₂; Z = COCH₃ |
| 167 | A = I; Z = CONH₂ | A = CF(CF₃)₂; Z = CONH₂ |
| 168 | A = I; Z = CN | A = CF(CF₃)₂; Z = CN |
| 169 | A = I; Z = NH—BOC | A = CF(CF₃)₂; Z = NH—BOC |

### Examples 170-179: 2-Pentafluoroethyl-3-Substituted Pyridines

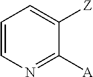

Procedure A is used to prepare the pentafluoroethyl derivative from the corresponding iodide.

| Example | Starting material | Product |
|---|---|---|
| 170 | A = I; Z = Cl | A = CF₂CF₃; Z = Cl |
| 171 | A = I; Z = OC₂H₅ | A = CF₂CF₃; Z = OC₂H₅ |
| 172 | A = I; Z = O—Bz | A = CF₂CF₃; Z = O—Bz |
| 173 | A = I; Z = Br | A = CF₂CF₃; Z = Br |
| 174 | A = I; Z = HC═O | A = CF₂CF₃; Z = HC═O |
| 175 | A = I; Z = CO₂CH₃ | A = CF₂CF₃; Z = CO₂CH₃ |
| 176 | A = I; Z = COCH₃ | A = CF₂CF₃; Z = COCH₃ |
| 177 | A = I; Z = CONH₂ | A = CF₂CF₃; Z = CONH₂ |
| 178 | A = I; Z = CN | A = CF₂CF₃; Z = CN |
| 179 | A = I; Z = NH—BOC | A = CF₂CF₃; Z = NH—BOC |

### Examples 180-189: 2-Heptafluoropropyl-3-Substituted Pyridines

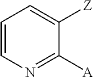

Procedure B is used to prepare the heptafluoropropyl derivative from the corresponding iodide.

| Example | Starting material | Product |
|---|---|---|
| 180 | A = I; Z = Cl | A = CF₂CF₂CF₃; Z = Cl |
| 181 | A = I; Z = OC₂H₅ | A = CF₂CF₂CF₃; Z = OC₂H₅ |
| 182 | A = I; Z = O—Bz | A = CF₂CF₂CF₃; Z = O—Bz |
| 183 | A = I; Z = Br | A = CF₂CF₂CF₃; Z = Br |
| 184 | A = I; Z = HC═O | A = CF₂CF₂CF₃ Z = HC═O |
| 185 | A = I; Z = CO₂CH₃ | A = CF₂CF₂CF₃; Z = CO₂CH₃ |
| 186 | A = I; Z = COCH₃ | A = CF₂CF₂CF₃; Z = COCH₃ |
| 187 | A = I; Z = CONH₂ | A = CF₂CF₂CF₃; Z = CONH₂ |
| 188 | A = I; Z = CN | A = CF₂CF₂CF₃; Z = CN |
| 189 | A = I; Z = NH—BOC | A = CF₂CF₂CF₃; Z = NH—BOC |

### Examples 190-199: 2-Nonafluorobutyl-3-Substituted Pyridines

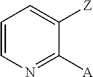

Procedure C is used to prepare the nonafluorobutyl derivative from the corresponding iodide.

| Example | Starting material | Product |
|---|---|---|
| 190 | A = I; Z = Cl | A = CF₂CF₂CF₂CF₃; Z = Cl |
| 191 | A = I; Z = OC₂H₅ | A = CF₂CF₂CF₂CF₃; Z = OC₂H₅ |
| 192 | A = I; Z = O—Bz | A = CF₂CF₂CF₂CF₃; Z = O—Bz |
| 193 | A = I; Z = Br | A = CF₂CF₂CF₂CF₃; Z = Br |
| 194 | A = I; Z = HC═O | A = CF₂CF₂CF₂CF₃; Z = HC═O |
| 195 | A = I; Z = CO₂CH₃ | A = CF₂CF₂CF₂CF₃; Z = CO₂CH₃ |
| 196 | A = I; Z = COCH₃ | A = CF₂CF₂CF₂CF₃; Z = COCH₃ |
| 197 | A = I; Z = CONH₂ | A = CF₂CF₂CF₂CF₃; Z = CONH₂ |
| 198 | A = I; Z = CN | A = CF₂CF₂CF₂CF₃; Z = CN |
| 199 | A = I; Z = NH—BOC | A = CF₂CF₂CF₂CF₃; Z = NH—BOC |

### Examples 200-209: 2-Difluoromethyl-3-Substituted Pyridines

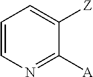

Procedure D is used to prepare the difluoromethyl derivative from the corresponding iodide.

| Example | Starting material | Product |
|---|---|---|
| 200 | A = I; Z = Cl | A = CHF₂; Z = Cl |
| 201 | A = I; Z = OC₂H₅ | A = CHF₂; Z = OC₂H₅ |
| 202 | A = I; Z = O—Bz | A = CHF₂; Z = O—Bz |
| 203 | A = I; Z = Br | A = CHF₂; Z = Br |
| 204 | A = I; Z = HC═O | A = CHF₂; Z = HC═O |
| 205 | A = I; Z = CO₂CH₃ | A = CHF₂; Z = CO₂CH₃ |
| 206 | A = I; Z = COCH₃ | A = CHF₂; Z = COCH₃ |
| 207 | A = I; Z = CONH₂ | A = CHF₂; Z = CONH₂ |
| 208 | A = I; Z = CN | A = CHF₂; Z = CN |
| 209 | A = I; Z = NH—BOC | A = CHF₂; Z = NH—BOC |

### Examples 210-219: 2-Heptafluoroisopropyl-3-Substituted Pyridines

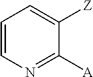

Procedure E is used to prepare the heptafluoroisopropyl derivative from the corresponding iodide.

| Example | Starting material | Product |
|---|---|---|
| 210 | A = I; Z = Cl | A = CF(CF₃)₂; Z = Cl |
| 211 | A = I; Z = OC₂H₅ | A = CF(CF₃)₂; Z = OC₂H₅ |
| 212 | A = I; Z = O—Bz | A = CF(CF₃)₂; Z = O—Bz |
| 213 | A = I; Z = Br | A = CF(CF₃)₂; Z = Br |
| 214 | A = I; Z = HC═O | A = CF(CF₃)₂; Z = HC═O |
| 215 | A = I; Z = CO₂CH₃ | A = CF(CF₃)₂; Z = CO₂CH₃ |
| 216 | A = I; Z = COCH₃ | A = CF(CF₃)₂; Z = COCH₃ |
| 217 | A = I; Z = CONH₂ | A = CF(CF₃)₂; Z = CONH₂ |
| 218 | A = I; Z = CN | A = CF(CF₃)₂; Z = CN |
| 219 | A = I; Z = NH—BOC | A = CF(CF₃)₂; Z = NH—BOC |

### Examples 220-229: 3-Pentafluoroethyl-2-Substituted Pyridines

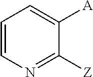

Procedure A is used to prepare the pentafluoroethyl derivative from the corresponding iodide or bromide.

| Example | Starting material | Product |
|---|---|---|
| 220 | A = Br; Z = Cl | A = CF₂CF₃; Z = Cl |
| 221 | A = Br; Z = OC₂H₅ | A = CF₂CF₃; Z = OC₂H₅ |
| 222 | A = I; Z = O—Bz | A = CF₂CF₃; Z = O—Bz |
| 223 | A = I; Z = Br | A = CF₂CF₃; Z = Br |
| 224 | A = Br; Z = HC═O | A = CF₂CF₃; Z = HC═O |
| 225 | A = Br; Z = CO₂CH₃ | A = CF₂CF₃; Z = CO₂CH₃ |
| 226 | A = Br; Z = COCH₃ | A = CF₂CF₃; Z = COCH₃ |
| 227 | A = Br; Z = CONH₂ | A = CF₂CF₃; Z = CONH₂ |
| 228 | A = Br; Z = CN | A = CF₂CF₃; Z = CN |
| 229 | A = I; Z = NH—BOC | A = CF₂CF₃; Z = NH—BOC |

### Examples 230-239: 3-Heptafluoropropyl-b 2-Substituted Pyridines

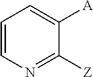

Procedure B is used to prepare the heptafluoropropyl derivative from the corresponding iodide or bromide.

| Example | Starting material | Product |
|---|---|---|
| 230 | A = Br; Z = Cl | A = CF₂CF₂CF₃; Z = Cl |
| 231 | A = I; Z = OC₂H₅ | A = CF₂CF₂CF₃; Z = OC₂H₅ |
| 232 | A = I; Z = O—Bz | A = CF₂CF₂CF₃; Z = O—Bz |
| 233 | A = I; Z = Br | A = CF₂CF₂CF₃; Z = Br |
| 234 | A = Br; Z = HC═O | A = CF₂CF₂CF₃; Z = HC═O |
| 235 | A = Br; Z = CO₂CH₃ | A = CF₂CF₂CF₃; Z = CO₂CH₃ |
| 236 | A = Br; Z = COCH₃ | A = CF₂CF₂CF₃; Z = COCH₃ |
| 237 | A = Br; Z = CONH₂ | A = CF₂CF₂CF₃; Z = CONH₂ |
| 238 | A = Br; Z = CN | A = CF₂CF₂CF₃; Z = CN |
| 239 | A = I; Z = NH—BOC | A = CF₂CF₂CF₃; Z = NH—BOC |

### Examples 240-249: 3-Nonafluorobutyl-2-Substituted Pyridines

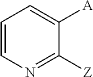

Procedure C is used to prepare the nonafluorobutyl derivative from the corresponding iodide or bromide.

| Example | Starting material | Product |
|---|---|---|
| 240 | A = Br; Z = Cl | A = CF₂CF₂CF₂CF₃; Z = Cl |
| 241 | A = Br; Z = OC₂H₅ | A = CF₂CF₂CF₂CF₃; Z = OC₂H₅ |
| 242 | A = I; Z = O—Bz | A = CF₂CF₂CF₂CF₃; Z = O—Bz |
| 243 | A = I; Z = Br | A = CF₂CF₂CF₂CF₃; Z = Br |
| 244 | A = Br; Z = HC═O | A = CF₂CF₂CF₂CF₃; Z = HC═O |
| 245 | A = Br; Z = CO₂CH₃ | A = CF₂CF₂CF₂CF₃; Z = CO₂CH₃ |
| 246 | A = Br; Z = COCH₃ | A = CF₂CF₂CF₂CF₃; Z = COCH₃ |
| 247 | A = Br; Z = CONH₂ | A = CF₂CF₂CF₂CF₃; Z = CONH₂ |
| 248 | A = Br; Z = CN | A = CF₂CF₂CF₂CF₃; Z = CN |
| 249 | A = I; Z = NH—BOC | A = CF₂CF₂CF₂CF₃; Z = NH—BOC |

### Examples 250-259: 3-Difluoromethyl-2-Substituted Pyridines

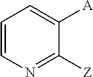

Procedure D is used to prepare the difluoromethyl derivative from the corresponding iodide or bromide.

| Example | Starting material | Product |
|---|---|---|
| 250 | A = Br; Z = Cl | A = CHF₂; Z = Cl |
| 251 | A = I; Z = OC₂H₅ | A = CHF₂; Z = OC₂H₅ |
| 252 | A = I; Z = O—Bz | A = CHF₂; Z = O—Bz |
| 253 | A = I; Z = Br | A = CHF₂; Z = Br |
| 254 | A = Br; Z = HC═O | A = CHF₂; Z = HC═O |
| 255 | A = Br; Z = CO₂CH₃ | A = CHF₂; Z = CO₂CH₃ |
| 256 | A = Br; Z = COCH₃ | A = CHF₂; Z = COCH₃ |
| 257 | A = Br; Z = CONH₂ | A = CHF₂; Z = CONH₂ |
| 258 | A = Br; Z = CN | A = CHF₂; Z = CN |
| 259 | A = I; Z = NH—BOC | A = CHF₂; Z = NH—BOC |

### Examples 260-269: 3-Heptafluoroisopropyl-2-Substituted Pyridines

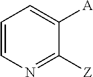

Procedure E is used to prepare the heptafluoroisopropyl derivative from the corresponding iodide or bromide.

| Example | Starting material | Product |
|---|---|---|
| 260 | A = Br; Z = Cl | A = CF(CF₃)₂; Z = Cl |
| 261 | A = I; Z = OC₂H₅ | A = CF(CF₃)₂; Z = OC₂H₅ |
| 262 | A = I; Z = O—Bz | A = CF(CF₃)₂; Z = O—Bz |
| 263 | A = I; Z = Br | A = CF(CF₃)₂; Z = Br |
| 264 | A = Br; Z = HC═O | A = CF(CF₃)₂; Z = HC═O |
| 265 | A = Br; Z = CO₂CH₃ | A = CF(CF₃)₂; Z = CO₂CH₃ |
| 266 | A = Br; Z = COCH₃ | A = CF(CF₃)₂; Z = COCH₃ |
| 267 | A = Br; Z = CONH₂ | A = CF(CF₃)₂; Z = CONH₂ |
| 268 | A = Br; Z = CN | A = CF(CF₃)₂; Z = CN |
| 269 | A = I; Z = NH—BOC | A = CF(CF₃)₂; Z = NH—BOC |

### Examples 270-279: 3-Pentafluoroethyl-4-Substituted Pyridines

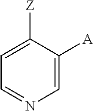

Procedure A is used to prepare the pentafluoroethyl derivative from the corresponding iodide or bromide.

| Example | Starting material | Product |
|---|---|---|
| 270 | A = Br; Z = Cl | A = CF₂CF₃; Z = Cl |
| 271 | A = I; Z = OC₂H₅ | A = CF₂CF₃; Z = OC₂H₅ |
| 272 | A = I; Z = O—Bz | A = CF₂CF₃; Z = O—Bz |
| 273 | A = I; Z = Br | A = CF₂CF₃; Z = Br |
| 274 | A = Br; Z = HC═O | A = CF₂CF₃; Z = HC═O |
| 275 | A = Br; Z = CO₂CH₃ | A = CF₂CF₃; Z = CO₂CH₃ |
| 276 | A = Br; Z = COCH₃ | A = CF₂CF₃; Z = COCH₃ |
| 277 | A = Br; Z = CONH₂ | A = CF₂CF₃; Z = CONH₂ |
| 278 | A = Br; Z = CN | A = CF₂CF₃; Z = CN |
| 279 | A = I; Z = NH—BOC | A = CF₂CF₃; Z = NH—BOC |

### Examples 280-289: 3-Heptafluoropropyl-4-Substituted Pyridines

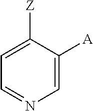

Procedure B is used to prepare the heptafluoropropyl derivative from the corresponding iodide or bromide.

| Example | Starting material | Product |
|---|---|---|
| 280 | A = Br; Z = Cl | A = CF₂CF₂CF₃; Z = Cl |
| 281 | A = I; Z = OC₂H₅ | A = CF₂CF₂CF₃; Z = OC₂H₅ |
| 282 | A = I; Z = O—Bz | A = CF₂CF₂CF₃; Z = O—Bz |
| 283 | A = I; Z = Br | A = CF₂CF₂CF₃; Z = Br |
| 284 | A = Br; Z = HC═O | A = CF₂CF₂CF₃; Z = HC═O |
| 285 | A = Br; Z = CO₂CH₃ | A = CF₂CF₂CF₃; Z = CO₂CH₃ |
| 286 | A = Br; Z = COCH₃ | A = CF₂CF₂CF₃; Z = COCH₃ |
| 287 | A = Br; Z = CONH₂ | A = CF₂CF₂CF₃; Z = CONH₂ |
| 288 | A = Br; Z = CN | A = CF₂CF₂CF₃; Z = CN |
| 289 | A = I; Z = NH—BOC | A = CF₂CF₂CF₃; Z = NH—BOC |

### Examples 298-299: 3-Nonafluorobutyl-4-Substituted Pyridines

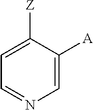

Procedure C is used to prepare the nonafluorobutyl derivative from the corresponding iodide or bromide.

| Example | Starting material | Product |
|---|---|---|
| 290 | A = Br; Z = Cl | A = CF₂CF₂CF₂CF₃; Z = Cl |
| 291 | A = I; Z = OC₂H₅ | A = CF₂CF₂CF₂CF₃; Z = OC₂H₅ |
| 292 | A = I; Z = O—Bz | A = CF₂CF₂CF₂CF₃; Z = O—Bz |
| 293 | A = I; Z = Br | A = CF₂CF₂CF₂CF₃; Z = Br |
| 294 | A = Br; Z = HC═O | A = CF₂CF₂CF₂CF₃; Z = HC═O |
| 295 | A = Br; Z = CO₂CH₃ | A = CF₂CF₂CF₂CF₃; Z = CO₂CH₃ |
| 296 | A = Br; Z = COCH₃ | A = CF₂CF₂CF₂CF₃; Z = COCH₃ |
| 297 | A = Br; Z = CONH₂ | A = CF₂CF₂CF₂CF₃; Z = CONH₂ |
| 298 | A = Br; Z = CN | A = CF₂CF₂CF₂CF₃; Z = CN |
| 299 | A = I; Z = NH—BOC | A = CF₂CF₂CF₂CF₃; Z = NH—BOC |

### Examples 390-309: 3-Difluoromethyl-4-Substituted Pyridines

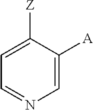

Procedure D is used to prepare the difluoromethyl derivative from the corresponding iodide or bromide.

| Example | Starting material | Product |
|---|---|---|
| 300 | A = Br; Z = Cl | A = CHF₂; Z = Cl |
| 301 | A = I; Z = OC₂H₅ | A = CHF₂; Z = OC₂H₅ |
| 302 | A = I; Z = O—Bz | A = CHF₂; Z = O—Bz |
| 303 | A = I; Z = Br | A = CHF₂; Z = Br |
| 304 | A = Br; Z = HC═O | A = CHF₂; Z = HC═O |
| 305 | A = Br; Z = CO₂CH₃ | A = CHF₂; Z = CO₂CH₃ |
| 306 | A = Br; Z = COCH₃ | A = CHF₂; Z = COCH₃ |
| 307 | A = Br; Z = CONH₂ | A = CHF₂; Z = CONH₂ |
| 308 | A = Br; Z = CN | A = CHF₂; Z = CN |
| 309 | A = I; Z = NH—BOC | A = CHF₂; Z = NH—BOC |

### Examples 310-319: 3-Heptafluoroisopropyl-4-Substituted Pyridines

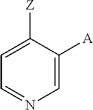

Procedure E is used to prepare the heptafluoroisopropyl derivative from the correspond kg iodide or bromide.

| Example | Starting material | Product |
|---|---|---|
| 310 | A = Br; Z = Cl | A = CF(CF₃)₂; Z = Cl |
| 311 | A = I; Z = OC₂H₅ | A = CF(CF₃)₂; Z = OC₂H₅ |
| 312 | A = I; Z = O—Bz | A = CF(CF₃)₂; Z = O—Bz |
| 313 | A = I; Z = Br | A = CF(CF₃)₂; Z = Br |
| 314 | A = Br; Z = HC═O | A = CF(CF₃)₂; Z = HC═O |
| 315 | A = Br; Z = CO₂CH₃ | A = CF(CF₃)₂; Z = CO₂CH₃ |
| 316 | A = Br; Z = COCH₃ | A = CF(CF₃)₂; Z = COCH₃ |
| 317 | A = Br; Z = CONH₂ | A = CF(CF₃)₂; Z = CONH₂ |
| 318 | A = Br; Z = CN | A = CF(CF₃)₂; Z = CN |
| 319 | A = I; Z = NH—BOC | A = CF(CF₃)₂; Z = NH—BOC |

### Examples 320-329: 3-Pentafluoroethyl-5-Substituted Pyridines

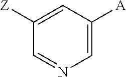

Procedure A is used to prepare the pentafluoroethyl derivative from the corresponding iodide or bromide.

| Example | Starting material | Product |
|---|---|---|
| 310 | A = Br; Z = Cl | A = CF(CF₃)₂; Z = Cl |
| 311 | A = I; Z = OC₂H₅ | A = CF(CF₃)₂; Z = OC₂H₅ |
| 312 | A = I; Z = O—Bz | A = CF(CF₃)₂; Z = O—Bz |
| 313 | A = I; Z = Br | A = CF(CF₃)₂; Z = Br |
| 314 | A = Br; Z = HC═O | A = CF(CF₃)₂; Z = HC═O |
| 315 | A = Br; Z = CO₂CH₃ | A = CF(CF₃)₂; Z = CO₂CH₃ |
| 316 | A = Br; Z = COCH₃ | A = CF(CF₃)₂; Z = COCH₃ |
| 317 | A = Br; Z = CONH₂ | A = CF(CF₃)₂; Z = CONH₂ |
| 318 | A = Br; Z = CN | A = CF(CF₃)₂; Z = CN |
| 319 | A = I; Z = NH—BOC | A = CF(CF₃)₂; Z = NH—BOC |

### Examples 330-339: 3-Heptafluoropropyl-b 5-Substituted Pyridines

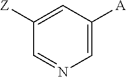

Procedure B is used to prepare the heptafluoropropyl derivative from the corresponding iodide or bromide.

| Example | Starting material | Product |
|---|---|---|
| 330 | A = Br; Z = Cl | A = CF₂CF₂CF₃; Z = Cl |
| 331 | A = I; Z = OC₂H₅ | A = CF₂CF₂CF₃; Z = OC₂H₅ |
| 332 | A = I; Z = O—Bz | A = CF₂CF₂CF₃; Z = O—Bz |
| 333 | A = I; Z = Br | A = CF₂CF₂CF₃; Z = Br |
| 334 | A = Br; Z = HC═O | A = CF₂CF₂CF₃; Z = HC═O |
| 335 | A = Br; Z = CO₂CH₃ | A = CF₂CF₂CF₃; Z = CO₂CH₃ |
| 336 | A = Br; Z = COCH₃ | A = CF₂CF₂CF₃; Z = COCH₃ |
| 337 | A = Br; Z = CONH₂ | A = CF₂CF₂CF₃; Z = CONH₂ |
| 338 | A = Br; Z = CN | A = CF₂CF₂CF₃; Z = CN |
| 339 | A = I; Z = NH—BOC | A = CF₂CF₂CF₃; Z = NH—BOC |

### Examples 340-349: 3-Nonafluorobutyl-5-Substituted Pyridines

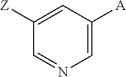

Procedure C is used to prepare the nonafluorobutyl derivative from the corresponding iodide or bromide.

| Example | Starting material | Product |
|---|---|---|
| 340 | A = Br; Z = Cl | A = CF₂CF₂CF₂CF₃; Z = Cl |
| 341 | A = I; Z = OC₂H₅ | A = CF₂CF₂CF₂CF₃; Z = OC₂H₅ |
| 342 | A = I; Z = O—Bz | A = CF₂CF₂CF₂CF₃; Z = O—Bz |
| 343 | A = I; Z = Br | A = CF₂CF₂CF₂CF₃; Z = Br |
| 344 | A = Br; Z = HC═O | A = CF₂CF₂CF₂CF₃; Z = HC═O |
| 345 | A = Br; Z = CO₂CH₃ | A = CF₂CF₂CF₂CF₃; Z = CO₂CH₃ |
| 346 | A = Br; Z = COCH₃ | A = CF₂CF₂CF₂CF₃; Z = COCH₃ |
| 347 | A = Br; Z = CONH₂ | A = CF₂CF₂CF₂CF₃; Z = CONH₂ |
| 348 | A = Br; Z = CN | A = CF₂CF₂CF₂CF₃; Z = CN |
| 349 | A = I; Z = NH—BOC | A = CF₂CF₂CF₂CF₃; Z = NH—BOC |

### Examples 350-359: 3-Difluoromethyl-5-Substituted Pyridines

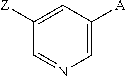

Procedure D is used to prepare the difluoromethyl derivative from the corresponding iodide or bromide.

| Example | Starting material | Product |
|---|---|---|
| 350 | A = Br; Z = Cl | A = CHF₂; Z = Cl |
| 351 | A = I; Z = OC₂H₅ | A = CHF₂; Z = OC₂H₅ |
| 352 | A = I; Z = O—Bz | A = CHF₂; Z = O—Bz |
| 353 | A = I; Z = Br | A = CHF₂; Z = Br |
| 354 | A = Br; Z = HC═O | A = CHF₂; Z = HC═O |
| 355 | A = Br; Z = CO₂CH₃ | A = CHF₂; Z = CO₂CH₃ |
| 356 | A = Br; Z = COCH₃ | A = CHF₂; Z = COCH₃ |
| 357 | A = Br; Z = CONH₂ | A = CHF₂; Z = CONH₂ |
| 358 | A = Br; Z = CN | A = CHF₂; Z = CN |
| 359 | A = I; Z = NH—BOC | A = CHF₂; Z = NH—BOC |

### Examples 360-369: 3-Heptafluoroisopropyl-5-Substituted Pyridines

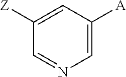

Procedure E is used to prepare the heptafluoroisopropyl derivative from the corresponding iodide or bromide.

| Example | Starting material | Product |
|---|---|---|
| 360 | A = Br; Z = Cl | A = CF(CF₃)₂; Z = Cl |
| 361 | A = I; Z = OC₂H₅ | A = CF(CF₃)₂; Z = OC₂H₅ |
| 362 | A = I; Z = O—Bz | A = CF(CF₃)₂; Z = O—Bz |
| 363 | A = I; Z = Br | A = CF(CF₃)₂; Z = Br |
| 364 | A = Br; Z = HC═O | A = CF(CF₃)₂; Z = HC═O |
| 365 | A = Br; Z = CO₂CH₃ | A = CF(CF₃)₂; Z = CO₂CH₃ |
| 366 | A = Br; Z = COCH₃ | A = CF(CF₃)₂; Z = COCH₃ |
| 367 | A = Br; Z = CONH₂ | A = CF(CF₃)₂; Z = CONH₂ |
| 368 | A = Br; Z = CN | A = CF(CF₃)₂; Z = CN |
| 369 | A = I; Z = NH—BOC | A = CF(CF₃)₂; Z = NH—BOC |

### Examples 370-379: 3-Pentafluoroethyl-4-Substituted Pyridines

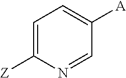

Procedure A is used to prepare the pentafluoroethyl derivative from the corresponding iodide or bromide.

| Example | Starting material | Product |
|---|---|---|
| 370 | A = Br; Z = Cl | A = CF₂CF₃; Z = Cl |
| 371 | A = I; Z = OC₂H₅ | A = CF₂CF₃; Z = OC₂H₅ |
| 372 | A = I; Z = O—Bz | A = CF₂CF₃; Z = O—Bz |
| 373 | A = I; Z = Br | A = CF₂CF₃; Z = Br |
| 374 | A = Br; Z = HC═O | A = CF₂CF₃; Z = HC═O |
| 375 | A = Br; Z = CO₂CH₃ | A = CF₂CF₃; Z = CO₂CH₃ |
| 376 | A = Br; Z = COCH₃ | A = CF₂CF₃; Z = COCH₃ |
| 377 | A = Br; Z = CONH₂ | A = CF₂CF₃; Z = CONH₂ |
| 378 | A = Br; Z = CN | A = CF₂CF₃; Z = CN |
| 379 | A = I; Z = NH—BOC | A = CF₂CF₃; Z = NH—BOC |

### Examples 380-389: 3-Heptafluoropropyl-6-Substituted Pyridines

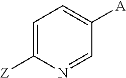

Procedure B is used to prepare the heptafluoropropyl derivative from the corresponding iodide or bromide.

| Example | Starting material | Product |
|---|---|---|
| 380 | A = Br; Z = Cl | A = CF₂CF₂CF₃; Z = Cl |
| 381 | A = I; Z = OC₂H₅ | A = CF₂CF₂CF₃; Z = OC₂H₅ |
| 382 | A = I; Z = O—Bz | A = CF₂CF₂CF₃; Z = O—Bz |
| 383 | A = I; Z = Br | A = CF₂CF₂CF₃; Z = Br |
| 384 | A = Br; Z = HC═O | A = CF₂CF₂CF₃; Z = HC═O |
| 385 | A = Br; Z = CO₂CH₃ | A = CF₂CF₂CF₃; Z = CO₂CH₃ |
| 386 | A = Br; Z = COCH₃ | A = CF₂CF₂CF₃; Z = COCH₃ |
| 387 | A = Br; Z = CONH₂ | A = CF₂CF₂CF₃; Z = CONH₂ |
| 388 | A = Br; Z = CN | A = CF₂CF₂CF₃; Z = CN |
| 389 | A = I; Z = NH—BOC | A = CF₂CF₂CF₃; Z = NH—BOC |

### Examples 390-399: 3-Nonafluorobutyl-6-Substituted Pyridines

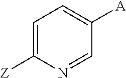

Procedure C is used to prepare the nonafluorobutyl derivative from the corresponding iodide or bromide.

| Example | Starting material | Product |
|---|---|---|
| 390 | A = Br; Z = Cl | A = CF₂CF₂CF₂CF₃; Z = Cl |
| 391 | A = I; Z = OC₂H₅ | A = CF₂CF₂CF₂CF₃; Z = OC₂H₅ |
| 392 | A = I; Z = O—Bz | A = CF₂CF₂CF₂CF₃; Z = O—Bz |
| 393 | A = I; Z = Br | A = CF₂CF₂CF₂CF₃; Z = Br |
| 394 | A = Br; Z = HC═O | A = CF₂CF₂CF₂CF₃; Z = HC═O |
| 395 | A = Br; Z = CO₂CH₃ | A = CF₂CF₂CF₂CF₃; Z = CO₂CH₃ |
| 396 | A = Br; Z = COCH₃ | A = CF₂CF₂CF₂CF₃; Z = COCH₃ |
| 397 | A = Br; Z = CONH₂ | A = CF₂CF₂CF₂CF₃; Z = CONH₂ |
| 398 | A = Br; Z = CN | A = CF₂CF₂CF₂CF₃; Z = CN |
| 399 | A = I; Z = NH—BOC | A = CF₂CF₂CF₂CF₃; Z = NH—BOC |

### Examples 400-409: 3-Difluoromethyl-6-Substituted Pyridines

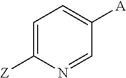

Procedure D is used to prepare the difluoromethyl derivative from the corresponding iodide or bromide.

| Example | Starting material | Product |
|---|---|---|
| 400 | A = Br; Z = Cl | A = CHF₂; Z = Cl |
| 401 | A = I; Z = OC₂H₅ | A = CHF₂; Z = OC₂H₅ |
| 402 | A = I; Z = O—Bz | A = CHF₂; Z = O—Bz |
| 403 | A = I; Z = Br | A = CHF₂; Z = Br |
| 404 | A = Br; Z = HC═O | A = CHF₂; Z = HC═O |
| 405 | A = Br; Z = CO₂CH₃ | A = CHF₂; Z = CO₂CH₃ |
| 406 | A = Br; Z = COCH₃ | A = CHF₂; Z = COCH₃ |
| 407 | A = Br; Z = CONH₂ | A = CHF₂; Z = CONH₂ |
| 408 | A = Br; Z = CN | A = CHF₂; Z = CN |
| 409 | A = I; Z = NH—BOC | A = CHF₂; Z = NH—BOC |

### Examples 410-419: 3-Heptafluoroisopropyl-6-Substituted Pyridines

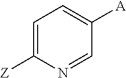

Procedure E is used to prepare the heptafluoroisopropyl derivative from the corresponding iodide or bromide.

| Example | Starting material | Product |
|---|---|---|
| 410 | A = Br; Z = Cl | A = CF(CF₃)₂; Z = Cl |
| 411 | A = I; Z = OC₂H₅ | A = CF(CF₃)₂; Z = OC₂H₅ |
| 412 | A = I; Z = O—Bz | A = CF(CF₃)₂; Z = O—Bz |
| 413 | A = I; Z = Br | A = CF(CF₃)₂; Z = Br |
| 414 | A = Br; Z = HC═O | A = CF(CF₃)₂; Z = HC═O |
| 415 | A = Br; Z = CO₂CH₃ | A = CF(CF₃)₂; Z = CO₂CH₃ |
| 416 | A = Br; Z = COCH₃ | A = CF(CF₃)₂; Z = COCH₃ |
| 417 | A = Br; Z = CONH₂ | A = CF(CF₃)₂; Z = CONH₂ |
| 418 | A = Br; Z = CN | A = CF(CF₃)₂; Z = CN |
| 419 | A = I; Z = NH—BOC | A = CF(CF₃)₂; Z = NH—BOC |

### Examples 420-429: 4-Pentafluoroethyl-2-Substituted Pyridines

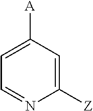

Procedure A is used to prepare the pentafluoroethyl derivative from the corresponding iodide or bromide.

| Example | Starting material | Product |
|---|---|---|
| 420 | A = Br; Z = Cl | A = CF₂CF₃; Z = Cl |
| 421 | A = I; Z = OC₂H₅ | A = CF₂CF₃; Z = OC₂H₅ |
| 422 | A = I; Z = O—Bz | A = CF₂CF₃; Z = O—Bz |
| 423 | A = I; Z = Br | A = CF₂CF₃; Z = Br |
| 424 | A = Br; Z = HC═O | A = CF₂CF₃; Z = HC═O |
| 425 | A = Br; Z = CO₂CH₃ | A = CF₂CF₃; Z = CO₂CH₃ |
| 426 | A = Br; Z = COCH₃ | A = CF₂CF₃; Z = COCH₃ |
| 427 | A = Br; Z = CONH₂ | A = CF₂CF₃; Z = CONH₂ |
| 428 | A = Br; Z = CN | A = CF₂CF₃; Z = CN |
| 429 | A = I; Z = NH—BOC | A = CF₂CF₃; Z = NH—BOC |

### Examples 430-439: 4-Heptafluoropropyl-2-Substituted Pyridines

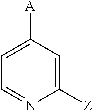

Procedure B is used to prepare the heptafluoropropyl derivative from the corresponding iodide or bromide.

| Example | Starting material | Product |
|---|---|---|
| 430 | A = Br; Z = Cl | A = CF₂CF₂CF₃; Z = Cl |
| 431 | A = I; Z = OC₂H₅ | A = CF₂CF₂CF₃; Z = OC₂H₅ |
| 432 | A = I; Z = O—Bz | A = CF₂CF₂CF₃; Z = O—Bz |
| 433 | A = I; Z = Br | A = CF₂CF₂CF₃; Z = Br |
| 434 | A = Br; Z = HC═O | A = CF₂CF₂CF₃; Z = HC═O |
| 435 | A = Br; Z = CO₂CH₃ | A = CF₂CF₂CF₃; Z = CO₂CH₃ |
| 436 | A = Br; Z = COCH₃ | A = CF₂CF₂CF₃; Z = COCH₃ |
| 437 | A = Br; Z = CONH₂ | A = CF₂CF₂CF₃; Z = CONH₂ |
| 438 | A = Br; Z = CN | A = CF₂CF₂CF₃; Z = CN |
| 439 | A = I; Z = NH—BOC | A = CF₂CF₂CF₃; Z = NH—BOC |

### Examples 440-449: 4-Nonafluorobutyl-2-Substituted Pyridines

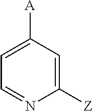

Procedure C is used to prepare the nonafluorobutyl derivative from the corresponding iodide or bromide.

| Example | Starting material | Product |
|---|---|---|
| 440 | A = Br; Z = Cl | A = CF₂CF₂CF₂CF₃; Z = Cl |
| 441 | A = I; Z = OC₂H₅ | A = CF₂CF₂CF₂CF₃; Z = OC₂H₅ |
| 442 | A = I; Z = O—Bz | A = CF₂CF₂CF₂CF₃; Z = O—Bz |
| 443 | A = I; Z = Br | A = CF₂CF₂CF₂CF₃; Z = Br |
| 444 | A = Br; Z = HC═O | A = CF₂CF₂CF₂CF₃; Z = HC═O |
| 445 | A = Br; Z = CO₂CH₃ | A = CF₂CF₂CF₂CF₃; Z = CO₂CH₃ |
| 446 | A = Br; Z = COCH₃ | A = CF₂CF₂CF₂CF₃; Z = COCH₃ |
| 447 | A = Br; Z = CONH₂ | A = CF₂CF₂CF₂CF₃; Z = CONH₂ |
| 448 | A = Br; Z = CN | A = CF₂CF₂CF₂CF₃; Z = CN |
| 449 | A = I; Z = NH—BOC | A = CF₂CF₂CF₂CF₃; Z = NH—BOC |

### Examples 450-459: 4-Difluoromethyl-2-Substituted Pyridines

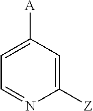

Procedure D is used to prepare the difluoromethyl derivative from the corresponding iodide or bromide.

| Example | Starting material | Product |
|---|---|---|
| 450 | A = Br; Z = Cl | A = CHF₂; Z = Cl |
| 451 | A = I; Z = OC₂H₅ | A = CHF₂; Z = OC₂H₅ |
| 452 | A = I; Z = O—Bz | A = CHF₂; Z = O—Bz |
| 453 | A = I; Z = Br | A = CHF₂; Z = Br |
| 454 | A = Br; Z = HC═O | A = CHF₂; Z = HC═O |
| 455 | A = Br; Z = CO₂CH₃ | A = CHF₂; Z = CO₂CH₃ |
| 456 | A = Br; Z = COCH₃ | A = CHF₂; Z = COCH₃ |
| 457 | A = Br; Z = CONH₂ | A = CHF₂; Z = CONH₂ |
| 458 | A = Br; Z = CN | A = CHF₂; Z = CN |
| 459 | A = I; Z = NH—BOC | A = CHF₂; Z = NH—BOC |

### Examples 460-469: 4-Heptafluoroisopropyl-2-Substituted Pyridines

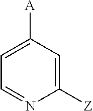

Procedure E is used to prepare the heptafluoroisopropyl derivative from the corresponding iodide or bromide.

| Example | Starting material | Product |
|---|---|---|
| 460 | A = Br; Z = Cl | A = CF(CF₃)₂; Z = Cl |
| 461 | A = I; Z = OC₂H₅ | A = CF(CF₃)₂; Z = OC₂H₅ |
| 462 | A = I; Z = O—Bz | A = CF(CF₃)₂; Z = O—Bz |
| 463 | A = I; Z = Br | A = CF(CF₃)₂; Z = Br |
| 464 | A = Br; Z = HC═O | A = CF(CF₃)₂; Z = HC═O |
| 465 | A = Br; Z = CO₂CH₃ | A = CF(CF₃)₂; Z = CO₂CH₃ |
| 466 | A = Br; Z = COCH₃ | A = CF(CF₃)₂; Z = COCH₃ |
| 467 | A = Br; Z = CONH₂ | A = CF(CF₃)₂; Z = CONH₂ |
| 468 | A = Br; Z = CN | A = CF(CF₃)₂; Z = CN |
| 469 | A = I; Z = NH—BOC | A = CF(CF₃)₂; Z = NH—BOC |

### Examples 470-479: 4-Pentafluoroethyl-3-Substituted Pyridines

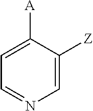

Procedure A is used to prepare the pentafluoroethyl derivative from the corresponding iodide or bromide.

| Example | Starting material | Product |
|---|---|---|
| 470 | A = Br; Z = Cl | A = CF₂CF₃; Z = Cl |
| 471 | A = I; Z = OC₂H₅ | A = CF₂CF₃; Z = OC₂H₅ |
| 472 | A = I; Z = O—Bz | A = CF₂CF₃; Z = O—Bz |
| 473 | A = I; Z = Br | A = CF₂CF₃; Z = Br |
| 474 | A = Br; Z = HC═O | A = CF₂CF₃; Z = HC═O |
| 475 | A = Br; Z = CO₂CH₃ | A = CF₂CF₃; Z = CO₂CH₃ |
| 476 | A = Br; Z = COCH₃ | A = CF₂CF₃; Z = COCH₃ |
| 477 | A = Br; Z = CONH₂ | A = CF₂CF₃; Z = CONH₂ |
| 478 | A = Br; Z = CN | A = CF₂CF₃; Z = CN |
| 479 | A = I; Z = NH—BOC | A = CF₂CF₃; Z = NH—BOC |

### Examples 480-489: 4-Heptafluoropropyl-3-Substituted Pyridines

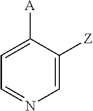

Procedure B is used to prepare the heptafluoropropyl derivative from the corresponding iodide or bromide.

| Example | Starting material | Product |
|---|---|---|
| 480 | A = Br; Z = Cl | A = CF₂CF₂CF₃; Z = Cl |
| 481 | A = I; Z = OC₂H₅ | A = CF₂CF₂CF₃; Z = OC₂H₅ |
| 482 | A = I; Z = O—Bz | A = CF₂CF₂CF₃; Z = O—Bz |
| 483 | A = I; Z = Br | A = CF₂CF₂CF₃; Z = Br |
| 484 | A = Br; Z = HC═O | A = CF₂CF₂CF₃; Z = HC═O |
| 485 | A = Br; Z = CO₂CH₃ | A = CF₂CF₂CF₃; Z = CO₂CH₃ |
| 486 | A = Br; Z = COCH₃ | A = CF₂CF₂CF₃; Z = COCH₃ |
| 487 | A = Br; Z = CONH₂ | A = CF₂CF₂CF₃; Z = CONH₂ |
| 488 | A = Br; Z = CN | A = CF₂CF₂CF₃; Z = CN |
| 489 | A = I; Z = NH—BOC | A = CF₂CF₂CF₃; Z = NH—BOC |

### Examples 490-499: 4-Nonafluorobutyl-3-Substituted Pyridines

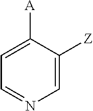

Procedure C is used to prepare the nonafluorobutyl derivative from the corresponding iodide or bromide.

| Example | Starting material | Product |
|---|---|---|
| 490 | A = Br; Z = Cl | A = CF₂CF₂CF₂CF₃; Z = Cl |
| 491 | A = I; Z = OC₂H₅ | A = CF₂CF₂CF₂CF₃; Z = OC₂H₅ |
| 492 | A = I; Z = O—Bz | A = CF₂CF₂CF₂CF₃; Z = O—Bz |
| 493 | A = I; Z = Br | A = CF₂CF₂CF₂CF₃; Z = Br |
| 494 | A = Br; Z = HC═O | A = CF₂CF₂CF₂CF₃; Z = HC═O |
| 495 | A = Br; Z = CO₂CH₃ | A = CF₂CF₂CF₂CF₃; Z = CO₂CH₃ |
| 496 | A = Br; Z = COCH₃ | A = CF₂CF₂CF₂CF₃; Z = COCH₃ |
| 497 | A = Br; Z = CONH₂ | A = CF₂CF₂CF₂CF₃; Z = CONH₂ |
| 498 | A = Br; Z = CN | A = CF₂CF₂CF₂CF₃; Z = CN |
| 499 | A = I; Z = NH—BOC | A = CF₂CFCF₂₂CF₃; Z = NH—BOC |

### Example 500-589: 4-Difluoromethyl-3-Substituted Pyridines

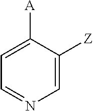

Procedure D is used to prepare the difluoromethyl derivative from the corresponding iodide or bromide.

| Example | Starting material | Product |
|---|---|---|
| 500 | A = Br; Z = Cl | A = CHF₂; Z = Cl |
| 501 | A = I; Z = OC₂H₅ | A = CHF₂; Z = OC₂H₅ |
| 502 | A = I; Z = O—Bz | A = CHF₂; Z = O—Bz |
| 503 | A = I; Z = Br | A = CHF₂; Z = Br |
| 504 | A = Br; Z = HC═O | A = CHF₂; Z = HC═O |
| 505 | A = Br; Z = CO₂CH₃ | A = CHF₂; Z = CO₂CH₃ |
| 506 | A = Br; Z = COCH₃ | A = CHF₂; Z = COCH₃ |
| 507 | A = Br; Z = CONH₂ | A = CHF₂; Z = CONH₂ |
| 508 | A = Br; Z = CN | A = CHF₂; Z = CN |
| 509 | A = I; Z = NH—BOC | A = CHF₂; Z = NH—BOC |

### Examples 510-519: 4-Heptafluoroisopropyl-3-Substituted Pyridines

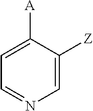

Procedure E is used to prepare the heptafluoroisopropyl derivative from the corresponding iodide or bromide.

| Example | Starting material | Product |
|---|---|---|
| 510 | A = Br; Z = Cl | A = CF(CF₃)₂; Z = Cl |
| 511 | A = I; Z = OC₂H₅ | A = CF(CF₃)₂; Z = OC₂H₅ |
| 512 | A = I; Z = O—Bz | A = CF(CF₃)₂; Z = O—Bz |
| 513 | A = I; Z = Br | A = CF(CF₃)₂; Z = Br |
| 514 | A = Br; Z = HC═O | A = CF(CF₃)₂; Z = HC═O |
| 515 | A = Br; Z = CO₂CH₃ | A = CF(CF₃)₂; Z = CO₂CH₃ |
| 516 | A = Br; Z = COCH₃ | A = CF(CF₃)₂; Z = COCH₃ |
| 517 | A = Br; Z = CONH₂ | A = CF(CF₃)₂; Z = CONH₂ |
| 518 | A = Br; Z = CN | A = CF(CF₃)₂; Z = CN |
| 519 | A = I; Z = NH—BOC | A = CF(CF₃)₂; Z = NH—BOC |

### Examples 520-529: 3-Pentafluoroethyl-2,6-Disubstituted Pyridines

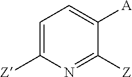

Procedure A is used to prepare the pentafluoroethyl derivative from the corresponding iodide or bromide.

| Example | Starting material | Product |
|---|---|---|
| 520 | A = Br; Z = Cl; Z’ = Cl | A = CF₂CF₃; Z = Cl; Z’ = Cl |
| 521 | A = I; Z = Br; Z’ = Br | A = CF₂CF₃; Z = Br; Z’ = Br |
| 522 | A = I; Z = Cl; Z’ = HC═O | A = CF₂CF₃; Z = Cl; Z’ = HC═O |
| 523 | A = I; Z = O—Bz; Z’ = Cl | A = CF₂CF₃; Z = O—Bz; Z’ = Cl |
| 524 | A = I; Z = Cl; Z’ = CO₂CH₃ | A = CF₂CF₃; Z = Cl; Z’ = CO₂CH₃ |
| 525 | A = I; Z = Br; Z’ = CONH₂ | A = CF₂CF₃; Z = Br; Z’ = CONH₂ |
| 526 | A = I; Z = NH—BOC; | A = CF₂CF₃; Z = NH—BOC; |
| | Z’ = Cl | Z’ = Cl |
| 527 | A = I; Z = Br; Z’ = CN | A = CF₂CF₃; Z = Br; Z’ = CN |
| 528 | A = I; Z = NH—BOC; | A = CF₂CF₃; Z = NH—BOC; |
| | Z’ = Br | Z’ = Br |
| 529 | A = I; Z = Cl; Z’ = CN | A = CF₂CF₃; Z = Cl; Z’ = CN |

### Examples 530-539: 3-Heptafluoropropyl-2,6-Disubstituted Pyridines

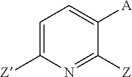

Procedure B is used to prepare the heptafluoropropyl derivative irons the corresponding iodide or bromide.

| Ex- | | |
|---|---|---|
| ample | Starting material | Product |
| 530 | A = Br; Z = Cl; Z’ = Cl | A = CF₂CF₂CF₃; Z = Cl; Z’ = Cl |
| 531 | A = I; Z = Br; Z’ = Br | A = CF₂CF₂CF₃; Z = Br; Z’ = Br |
| 532 | A = I; Z = Cl; Z’ = HC═O | A = CF₂CF₂CF₃; Z = Cl; Z’ = HC═O |
| 533 | A = I; Z = O—Bz; Z’ = Cl | A = CF₂CF₂CF₃; Z = O—Bz; Z’ = Cl |
| 534 | A = I; Z = Cl; Z’ = CO₂CH₃ | A = CF₂CF₂CF₃; Z = Cl; Z’ = CO₂CH₃ |
| 535 | A = I; Z = Br; Z’ = CONH₂ | A = CF₂CF₂CF₃; Z = Br; Z’ = CONH₂ |
| 536 | A = I; Z = NH—BOC; | A = CF₂CF₂CF₃; Z = NH—BOC; |
| | Z’ = Cl | Z’ = Cl |
| 537 | A = I; Z = Br; Z’ = CN | A = CF₂CF₂CF₃; Z = Br; Z’ = CN |
| 538 | A = I; Z = NH—BOC; | A = CF₂CF₂CF₃; Z = NH—BOC; |
| | Z’ = Br | Z’ = Br |
| 539 | A = I; Z = Cl; Z’ = CN | A = CF₂CF₂CF₃; Z = Cl; Z’ = CN |

### Examples 540-549: 3-Nonafluorobutyl-2,6-Disubstituted Pyridines

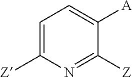

Procedure C is used to prepare the nonafluorobutyl derivative from the corresponding iodide or bromide.

| Ex- | | |
|---|---|---|
| ample | Starting material | Product |
| 540 | A = Br; Z = Cl; Z’ = Cl | A = CF₂CF₂CF₂CF₃; Z = Cl; Z’ = Cl |
| 541 | A = I; Z = Br; Z’ = Br | A = CF₂CF₂CF₂CF₃; Z = Br; Z’ = Br |
| 542 | A = I; Z = Cl; Z’ = HC═O | A = CF₂CF₂CF₂CF₃; Z = Cl; |
| | | Z’ = HC═O |
| 543 | A = I; Z = O—Bz; Z’ = Cl | A = CF₂CF₂CF₂CF₃; Z = O—Bz; |
| | | Z’ = Cl |
| 544 | A = I; Z = Cl; Z’ = CO₂CH₃ | A = CF₂CF₂CF₂CF₃; Z = Cl; |
| | | Z’ = CO₂CH₃ |
| 545 | A = I; Z = Br; Z’ = CONH₂ | A = CF₂CF₂CF₂CF₃; Z = Br; |
| | | Z’ = CONH₂ |
| 546 | A = I; Z = NH—BOC; | A = CF₂CF₂CF₂CF₃; Z = NH—BOC; |
| | Z’ = Cl | Z’ = Cl |
| 547 | A = I; Z = Br; Z’ = CN | A = CF₂CF₂CF₂CF₃; Z = Br; Z’ = CN |
| 548 | A = I; Z = NH—BOC; | A = CF₂CF₂CF₂CF₃; Z = NH—BOC; |
| | Z’ = Br | Z’ = Br |
| 549 | A = I; Z = Cl; Z’ = CN | A = CF₂CF₂CF₂CF₃; Z = Cl; Z’ = CN |

### Examples 550-559: 3-Difluoromethyl-2,6-Disubstituted Pyridines

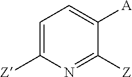

Procedure D is used to prepare the difluoromethyl derivative from the corresponding iodide or bromide.

| Ex- | | |
|---|---|---|
| ample | Starting material | Product |
| 550 | A = Br; Z = Cl; Z’ = Cl | A = CHF₂; Z = Cl; Z’ = Cl |
| 551 | A = I; Z = Br; Z’ = Br | A = CHF₂; Z = Br; Z’ = Br |
| 552 | A = I; Z = Cl; Z’ = HC═O | A = CHF₂; Z = Cl; Z’ = HC═O |
| 553 | A = I; Z = O—Bz; Z’ = Cl | A = CHF₂; Z = O—Bz; Z’ = Cl |
| 554 | A = I; Z = Cl; Z’ = CO₂CH₃ | A = CHF₂; Z = Cl; Z’ = CO₂CH₃ |
| 555 | A = I; Z = Br; Z’ = CONH₂ | A = CHF₂; Z = Br; Z’ = CONH₂ |
| 556 | A = I; Z = NH—BOC; Z’ = Cl | A = CHF₂; Z = NH—BOC; Z’ = Cl |
| 557 | A = I; Z = Br; Z’ = CN | A = CHF₂; Z = Br; Z’ = CN |
| 558 | A = I; Z = NH—BOC; Z’ = Br | A = CHF₂; Z = NH—BOC; Z’ = Br |
| 559 | A = I; Z = Cl; Z’ = CN | A = CHF₂; Z = Cl; Z’ = CN |

### Examples 560-569: 3-Heptafluoroisopropyl-2,6-Disubstituted Pyridines

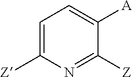

Procedure E is used to prepare the heptafluoroisopropyl derivative from the corresponding iodide or bromide.

| Ex- | | |
|---|---|---|
| ample | Starting material | Product |
| 560 | A = Br; Z = Cl; Z’ = Cl | A = CF(CF₃)₂; Z = Cl; Z’ = Cl |
| 561 | A = I; Z = Br; Z’ = Br | A = CF(CF₃)₂; Z = Br; Z’ = Br |
| 562 | A = I; Z = Cl; Z’ = HC═O | A = CF(CF₃)₂; Z = Cl; Z’ = HC═O |
| 563 | A = I; Z = O—Bz; Z’ = Cl | A = CF(CF₃)₂; Z = O—Bz; Z’ = Cl |
| 564 | A = I; Z = Cl; Z’ = CO₂CH₃ | A = CF(CF₃)₂; Z = Cl; Z’ = CO₂CH₃ |
| 565 | A = I; Z = Br; Z’ = CONH₂ | A = CF(CF₃)₂; Z = Br; Z’ = CONH₂ |
| 566 | A = I; Z = NH—BOC; | A = CF(CF₃)₂; Z = NH—BOC; |
| | Z’ = Cl | Z’ = Cl |
| 567 | A = I; Z = Br; Z’ = CN | A = CF(CF₃)₂; Z = Br; Z’ = CN |
| 568 | A = I; Z = NH—BOC; | A = CF(CF₃)₂; Z = NH—BOC; |
| | Z’ = Br | Z’ = Br |
| 569 | A = I; Z = Cl; Z’ = CN | A = CF(CF₃)₂; Z = Cl; Z’ = CN |

### Examples 570-579: 4-Pentafluoroethyl-2-Disubstituted Pyridines

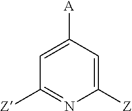

Procedure A is used to prepare the pentafluoroethyl derivative from the corresponding iodide or bromide.

| Ex- | | |
|---|---|---|
| ample | Starting material | Product |
| 570 | A = Br; Z = Cl; Z’ = Cl | A = CF₂CF₃; Z = Cl; Z’ = Cl |
| 571 | A = I; Z = Br; Z’ = Br | A = CF₂CF₃; Z = Br; Z’ = Br |
| 572 | A = I; Z = Cl; Z’ = HC═O | A = CF₂CF₃; Z = Cl; Z’ = HC═O |
| 573 | A = I; Z = O—Bz; Z’ = Cl | A = CF₂CF₃; Z = O—Bz; Z’ = Cl |
| 574 | A = I; Z = Cl; Z’ = CO₂CH₃ | A = CF₂CF₃; Z = Cl; Z’ = CO₂CH₃ |
| 575 | A = I; Z = Br; Z’ = CONH₂ | A = CF₂CF₃; Z = Br; Z’ = CONH₂ |
| 576 | A = I; Z = NH—BOC; | A = CF₂CF₃; Z = NH—BOC; |
| | Z’ = Cl | Z’ = Cl |
| 577 | A = I; Z = Br; Z’ = CN | A = CF₂CF₃; Z = Br; Z’ = CN |
| 578 | A = I; Z = NH—BOC; | A = CF₂CF₃; Z = NH—BOC; |
| | Z’ = Br | Z’ = Br |
| 579 | A = I; Z = Cl; Z’ = CN | A = CF₂CF₃; Z = Cl; Z’ = CN |

### Examples 580-589: 4-Heptafluoropropyl-2-Disubstituted Pyridines

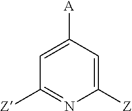

Procedure B is used to prepare the heptafluoropropyl derivative from the corresponding iodide or bromide.

| Ex- | | |
|---|---|---|
| ample | Starting material | Product |
| 580 | A = Br; Z = Cl; Z’ = Cl | A = CF₂CF₂CF₃; Z = Cl; Z’ = Cl |
| 581 | A = I; Z = Br; Z’ = Br | A = CF₂CF₂CF₃; Z = Br; Z’ = Br |
| 582 | A = I; Z = Cl; Z’ = HC═O | A = CF₂CF₂CF₃; Z = Cl; |
| | | Z’ = HC═O |
| 583 | A = I; Z = O—Bz; Z’ = Cl | A = CF₂CF₂CF₃; Z = O—Bz; |
| | | Z’ = Cl |
| 584 | A = I; Z = Cl; Z’ = CO₂CH₃ | A = CF₂CF₂CF₃; Z = Cl; |
| | | Z’ = CO₂CH₃ |
| 585 | A = I; Z = Br; Z’ = CONH₂ | A = CF₂CF₂CF₃; Z = Br; |
| | | Z’ = CONH₂ |
| 586 | A = I; Z = NH—BOC; | A = CF₂CF₂CF₃; Z = NH—BOC; |
| | Z’ = Cl | Z’ = Cl |
| 587 | A = I; Z = Br; Z’ = CN | A = CF₂CF₂CF₃; Z = Br; Z’ = CN |
| 588 | A = I; Z = NH—BOC; | A = CF₂CF₂CF₃; Z = NH—BOC; |
| | Z’ = Br | Z’ = Br |
| 589 | A = I; Z = Cl; Z’ = CN | A = CF₂CCF₂CF₃; Z = Cl; Z’ = CN |

### Examples 590-599: 4-Nonafluorobutyl-2,6-Disubstituted Pyridines

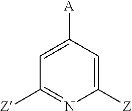

Procedure C is used to prepare the nonafluorobutyl derivative from the corresponding iodide or bromide.

| Ex- | | |
|---|---|---|
| ample | Starting material | Product |
| 590 | A = Br; Z = Cl; Z’ = Cl | A = CF₂CF₂CF₂CF₃; Z = Cl; Z’ = Cl |
| 591 | A = I; Z = Br; Z’ = Br | A = CF₂CF₂CF₂CF₃; Z = Br; Z’ = Br |
| 592 | A = I; Z = Cl; Z’ = HC═O | A = CF₂CF₂CF₂CF₃; Z = Cl; |
| | | Z’ = HC═O |
| 593 | A = I; Z = O—Bz; Z’ = Cl | A = CF₂CF₂CF₂CF₃; Z = O—Bz; |
| | | Z’ = Cl |
| 594 | A = I; Z = Cl; Z’ = CO₂CH₃ | A = CF₂CF₂CF₂CF₃; Z = Cl; |
| | | Z’ = CO₂CH₃ |
| 595 | A = I; Z = Br; Z’ = CONH₂ | A = CF₂CF₂CF₂CF₃; Z = Br; |
| | | Z’ = CONH₂ |
| 596 | A = I; Z = NH—BOC; | A = CF₂CF₂CF₂CF₃; Z = NH—BOC; |
| | Z’ = Cl | Z’ = Cl |
| 597 | A = I; Z = Br; Z’ = CN | A = CF₂CF₂CF₂CF₃; Z = Br; Z’ = CN |
| 598 | A = I; Z = NH—BOC; | A = CF₂CF₂CF₂CF₃; Z = NH—BOC; |
| | Z’ = Br | Z’ = Br |
| 599 | A = I; Z = Cl; Z’ = CN | A = CF₂CF₂CF₂CF₃; Z = Cl; Z’ = CN |

### Examples 606-609: 4-Difluoromethyl-2,6-Disubstituted Pyridines

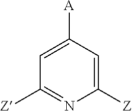

Procedure D is used to prepare the difluoromethyl derivative from the corresponding iodide or bromide.

| Ex- | | |
|---|---|---|
| ample | Starting material | Product |
| 600 | A = Br; Z = Cl; Z’ = Cl | A = CHF₂; Z = Cl; Z’ = Cl |
| 601 | A = I; Z = Br; Z’ = Br | A = CHF₂; Z = Br; Z’ = Br |
| 602 | A = I; Z = Cl; Z’ = HC═O | A = CHF₂; Z = Cl; Z’ = HC═O |
| 603 | A = I; Z = O—Bz; Z’ = Cl | A = CHF₂; Z = O—Bz; Z’ = Cl |
| 604 | A = I; Z = Cl; Z’ = CO₂CH₃ | A = CHF₂; Z = Cl; Z’ = CO₂CH₃ |
| 605 | A = I; Z = Br; Z’ = CONH₂ | A = CHF₂; Z = Br; Z’ = CONH₂ |
| 606 | A = I; Z = NH—BOC; Z’ = Cl | A = CHF₂; Z = NH—BOC; Z’ = Cl |
| 607 | A = I; Z = Br; Z’ = CN | A = CHF₂; Z = Br; Z’ = CN |
| 608 | A = I; Z = NH—BOC; Z’ = Br | A = CHF₂; Z = NH—BOC; Z’ = Br |
| 609 | A = I; Z = Cl; Z’ = CN | A = CHF₂; Z = Cl; Z’ = CN |

### Examples 610-619: 4-Heptafluoroisopropyl-2,6-Disubstituted Pyridines

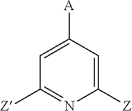

Procedure E is used to prepare the heptafluoroisopropyl derivative from the corresponding iodide or bromide.

| Ex- | | |
|---|---|---|
| ample | Starting material | Product |
| 610 | A = Br; Z = Cl; Z’ = Cl | A = CF(CF₃)₂; Z = Cl; Z’ = Cl |
| 611 | A = I; Z = Br; Z’ = Br | A = CF(CF₃)₂; Z = Br; Z’ = Br |
| 612 | A = I; Z = Cl; Z’ = HC═O | A = CF(CF₃)₂; Z = Cl; Z’ = HC═O |
| 613 | A = I; Z = O—Bz; Z’ = Cl | A = CF(CF₃)₂; Z = O—Bz; Z’ = Cl |
| 614 | A = I; Z = Cl; Z’ = CO₂CH₃ | A = CF(CF₃)₂; Z = Cl; Z’ = CO₂CH₃ |
| 615 | A = I; Z = Br; Z’ = CONH₂ | A = CF(CF₃)₂; Z = Br; Z’ = CONH₂ |
| 616 | A = I; Z = NH—BOC; | A = CF(CF₃)₂; Z = NH—BOC; |
| | Z’ = Cl | Z’ = Cl |
| 617 | A = I; Z = Br; Z’ = CN | A = CF(CF₃)₂; Z = Br; Z’ = CN |
| 618 | A = I; Z = NH—BOC; | A = CF(CF₃)₂; Z = NH—BOC; |
| | Z’ = Br | Z’ = Br |
| 619 | A = I; Z = Cl; Z’ = CN | A = CF(CF₃)₂; Z = Cl; Z’ = CN |

### Examples 620-627: 5-Pentafluoroethyl-2,4-Disubstituted Pyrimidines

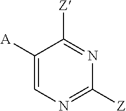

Procedure A is used to prepare the pentafluoroethyl derivative from the corresponding iodide.

| Example | Starting material | Product |
|---|---|---|
| 620 | A = I; Z = Cl; Z’ = Cl | A = C₂F₃; Z = Cl; Z’ = Cl |
| 621 | A = I; Z = OC₂H₅; Z’ = Cl | A = C₂F₃; Z = OC₂H₅; Z’ = Cl |
| 622 | A = I; Z = Cl; Z’ = OC₂H₅ | A = C₂F₃; Z = Cl; Z’ = OC₂H₅ |
| 623 | A = I; Z = O—Bz; Z’ = Cl | A = C₂F₃; Z = O—Bz; Z’ = Cl |
| 624 | A = I; Z = Cl; Z’ = O—Bz | A = C₂F₃; Z = Cl; Z’ = O—Bz |
| 625 | A = I; Z = Br; Z’ = Br | A = C₂F₃; Z = Br; Z’ = Br |
| 626 | A = I; Z = N(C₂H₅)₃; Z’ = Cl | A = C₂F₃; Z = N(C₂H₅)₃; Z’ = Cl |
| 627 | A = I; Z = Br; Z’ = Br | A = C₂F₃; Z = Br; Z’ = Br |

### Examples 628-635: 5-Heptafluoropropyl-2,4-Disubstituted Pyrimidines

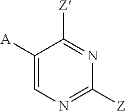

Procedure B is used to prepare the heptafluoropropyl derivative from the corresponding iodide.

| Ex- | | |
|---|---|---|
| ample | Starting material | Product |
| 628 | A = I; Z = Cl; Z’ = Cl | A = CF₂CF₂CF₃; Z = Cl; Z’ = Cl |
| 629 | A = I; Z = OC₂H₅; Z’ = Cl | A = CF₂CF₂CF₃; Z = OC₂H₅; Z’ = Cl |
| 630 | A = I; Z = Cl; Z’ = OC₂H₅ | A = CF₂CF₂CF₃; Z = Cl; Z’ = OC₂H₅ |
| 631 | A = I; Z = O—Bz; Z’ = Cl | A = CF₂CF₂CF₃; Z = O—Bz; Z’ = Cl |
| 632 | A = I; Z = Cl; Z’ = O—Bz | A = CF₂CF₂CF₃; Z = Cl; Z’ = O—Bz |
| 633 | A = I; Z = Br; Z’ = Br | A = CF₂CF₂CF₃; Z = Br; Z’ = Br |
| 634 | A = I; Z = N(C₂H₅)₃; | A = CF₂CF₂CF₃; Z = N(C₂H₅)₃; |
| | Z’ = Cl | Z’ = Cl |
| 635 | A = I; Z = Br; Z’ = Br | A = CF₂CF₂CF₃; Z = Br; Z’ = Br |

### Examples 636-643: 5-Nonafluorobutyl-2,4-Disubstituted Pyrimidines

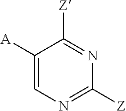

Procedure C is used to prepare the nonafluorobutyl derivative from the corresponding iodide or bromide.

| Ex- | | |
|---|---|---|
| am- | | |
| ple | Starting material | Product |
| 636 | A = I; Z = Cl; Z’ = Cl | A = CF₂CF₂CF₂CF₃; Z = Cl; Z’ = Cl |
| 637 | A = I; Z = OC₂H₅; Z’ = Cl | A = CF₂CF₂CF₂CF₃; Z = OC₂H₅; Z’ = Cl |
| 638 | A = I; Z = Cl; Z’ = OC₂H₅ | A = CF₂CF₂CF₂CF₃; Z = Cl; Z’ = OC₂H₅ |
| 639 | A = I; Z = O—Bz; Z’ = Cl | A = CF₂CF₂CF₂CF₃; Z = O—Bz; Z’ = Cl |
| 640 | A = I; Z = Cl; Z’ = O—Bz | A = CF₂CF₂CF₂CF₃; Z = Cl; Z’ = O—Bz |
| 641 | A = I; Z = Br; Z’ = Br | A = CF₂CF₂CF₂CF₃; Z = Br; Z’ = Br |
| 642 | A = I; Z = N(C₂H₅)₃; | A = CF₂CF₂CF₂CF₃; Z = N(C₂H₅)₃; |
| | Z’ = Cl | Z’ = Cl |
| 643 | A = I; Z = Br; Z’ = Br | A = CF₂CF₂CF₂CF₃; Z = Br; Z’ = Br |

### Examples 641-651: 5-Difluoromethyl-2,4-Disubstituted Pyrimidines

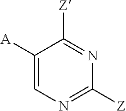

Procedure D is used to prepare the difluoromethyl derivative from the corresponding iodide.

| Example | Starting material | Product |
|---|---|---|
| 644 | A = I; Z = Cl; Z’ = Cl | A = CHF₂; Z = Cl; Z’ = Cl |
| 645 | A = I; Z = OC₂H₅; Z’ = Cl | A = CHF₂; Z = OC₂H₅; Z’ = Cl |
| 646 | A = I; Z = Cl; Z’ = OC₂H₅ | A = CHF₂; Z = Cl; Z’ = OC₂H₅ |
| 647 | A = I; Z = O—Bz; Z’ = Cl | A = CHF₂; Z = O—Bz; Z’ = Cl |
| 648 | A = I; Z = Cl; Z’ = O—Bz | A = CHF₂; Z = Cl; Z’ = O—Bz |
| 649 | A = I; Z = Br; Z’ = Br | A = CHF₂; Z = Br; Z’ = Br |
| 650 | A = I; Z = N(C₂H₅)₃; Z’ = Cl | A = CHF₂; Z = N(C₂H₅)₃; Z’ = Cl |
| 651 | A = I; Z = Br; Z’ = Br | A = CHF₂; Z = Br; Z’ = Br |

### Examples 652-659: 5-Heptafluoroisopropyl-2,4-Disubstituted Pyrimidines

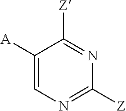

Procedure E is used to prepare the heptafluoroisopropyl derivative from the corresponding iodide.

| Ex- | | |
|---|---|---|
| ample | Starting material | Product |
| 652 | A = I; Z = Cl; Z’ = Cl | A = CF(CF₃)₂; Z = Cl; Z’ = Cl |
| 653 | A = I; Z = OC₂H₅; Z’ = Cl | A = CF(CF₃)₂; Z = OC₂H₅; Z’ = Cl |
| 654 | A = I; Z = Cl; Z’ = OC₂H₅ | A = CF(CF₃)₂; Z = Cl; Z’ = OC₂H₅ |
| 655 | A = I; Z = O—Bz; Z’ = Cl | A = CF(CF₃)₂; Z = O—Bz; Z’ = Cl |
| 656 | A = I; Z = Cl; Z’ = O—Bz | A = CF(CF₃)₂; Z = Cl; Z’ = O—Bz |
| 657 | A = I; Z = Br; Z’ = Br | A = CF(CF₃)₂; Z = Br; Z’ = Br |
| 658 | A = I; Z = N(C₂H₅)₃; Z’ = Cl | A = CF(CF₃)₂; Z = N(C₂H₅)₃; Z’ = Cl |
| 659 | A = I; Z = Br; Z’ = Br | A = CF(CF₃)₂; Z = Br; Z’ = Br |

### Examples 660-667: 2-Pentafluoroethyl-4,6-Disubstituted Pyrimidines

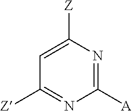

Procedure A is used to prepare the pentafluoroethyl derivative from the corresponding iodide.

| Example | Starting material | Product |
|---|---|---|
| 660 | A = I; Z = Cl; Z’ = Cl | A = C₂F₃; Z = Cl; Z’ = Cl |
| 661 | A = I; Z = OC₂H₅; Z’ = Cl | A = C₂F₃; Z = OC₂H₅; Z’ = Cl |
| 662 | A = I; Z = Cl; Z’ = OC₂H₅ | A = C₂F₃; Z = Cl; Z’ = OC₂H₅ |
| 663 | A = I; Z = O—Bz; Z’ = Cl | A = C₂F₃; Z = O—Bz; Z’ = Cl |
| 664 | A = I; Z = Cl; Z’ = O—Bz | A = C₂F₃; Z = Cl; Z’ = O—Bz |
| 665 | A = I; Z = Br; Z’ = Br | A = C₂F₃; Z = Br; Z’ = Br |
| 666 | A = I; Z = N(C₂H₅)₃; Z’ = Cl | A = C₂F₃; Z = N(C₂H₅)₃; Z’ = Cl |
| 667 | A = I; Z = Br; Z’ = Br | A = C₂F₃; Z = Br; Z’ = Br |

### Examples 668-675: 2-Heptafluoropropyl-4,6-Disubstituted Pyrimidines

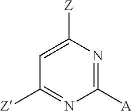

Procedure B is used to prepare the heptafluoropropyl derivative from the corresponding iodide.

| Ex- | | |
|---|---|---|
| ample | Starting material | Product |
| 668 | A = I; Z = Cl; Z’ = Cl | A = CF₂CF₂CF₃; Z = Cl; Z’ = Cl |
| 669 | A = I; Z = OC₂H₅; Z’ = Cl | A = CF₂CF₂CF₃; Z = OC₂H₅; Z’ = Cl |
| 670 | A = I; Z = Cl; Z’ = OC₂H₅ | A = CF₂CF₂CF₃; Z = Cl; Z’ = OC₂H₅ |
| 671 | A = I; Z = O—Bz; Z’ = Cl | A = CF₂CF₂CF₃; Z = O—Bz; Z’ = Cl |
| 672 | A = I; Z = Cl; Z’ = O—Bz | A = CF₂CF₂CF₃; Z = Cl; Z’ = O—Bz |
| 673 | A = I; Z = Br; Z’ = Br | A = CF₂CF₂CF₃; Z = Br; Z’ = Br |
| 674 | A = I; Z = N(C₂H₅)₃; | A = CF₂CF₂CF₃; Z = N(C₂H₅)₃; |
| | Z’ = Cl | Z’ = Cl |
| 675 | A = I; Z = Br; Z’ = Br | A = CF₂CF₂CF₃; Z = Br; Z’ = Br |

### Examples 676-683: 2-Nonafluorobutyl-4,6-Disubstituted Pyrimidines

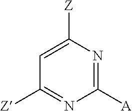

Procedure C is used to prepare the nonafluorobutyl derivative from the corresponding iodide.

| Ex- | | |
|---|---|---|
| am- | | |
| ple | Starting material | Product |
| 676 | A = I; Z = Cl; Z’ = Cl | A = CF₂CF₂CF₂CF₃; Z = Cl; Z’ = Cl |
| 677 | A = I; Z = OC₂H₅; Z’ = Cl | A = CF₂CF₂CF₂CF₃; Z = OC₂H₅; Z’ = Cl |
| 678 | A = I; Z = Cl; Z’ = OC₂H₅ | A = CF₂CF₂CF₂CF₃; Z = Cl; Z’ = OC₂H₅ |
| 679 | A = I; Z = O—Bz; Z’ = Cl | A = CF₂CF₂CF₂CF₃; Z = O—Bz; Z’ = Cl |
| 680 | A = I; Z = Cl; Z’ = O—Bz | A = CF₂CF₂CF₂CF₃; Z = Cl; Z’ = O—Bz |
| 681 | A = I; Z = Br; Z’ = Br | A = CF₂CF₂CF₂CF₃; Z = Br; Z’ = Br |
| 682 | A = I; Z = N(C₂H₅)₃; | A = CF₂CF₂CF₂CF₃; Z = N(C₂H₅)₃; |
| | Z’ = Cl | Z’ = Cl |
| 683 | A = I; Z = Br; Z’ = Br | A = CF₂CF₂CF₂CF₃; Z = Br; Z’ = Br |

### Examples 684-691: 2-Difluoromethyl-4,6-Disubstituted Pyrimidines

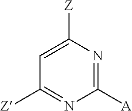

Procedure D is used to prepare the difluoromethyl derivative from the corresponding iodide.

| Example | Starting material | Product |
|---|---|---|
| 684 | A = I; Z = Cl; Z’ = Cl | A = CHF₂; Z = Cl; Z’ = Cl |
| 685 | A = I; Z = OC₂H₅; Z’ = Cl | A = CHF₂; Z = OC₂H₅; Z’ = Cl |
| 686 | A = I; Z = Cl; Z’ = OC₂H₅ | A = CHF₂; Z = Cl; Z’ = OC₂H₅ |
| 687 | A = I; Z = O—Bz; Z’ = Cl | A = CHF₂; Z = O—Bz; Z’ = Cl |
| 688 | A = I; Z = Cl; Z’ = O—Bz | A = CHF₂; Z = Cl; Z’ = O—Bz |
| 689 | A = I; Z = Br; Z’ = Br | A = CHF₂; Z = Br; Z’ = Br |
| 690 | A = I; Z = N(C₂H₅)₃; Z’ = Cl | A = CHF₂; Z = N(C₂H₅)₃; Z’ = Cl |
| 691 | A = I; Z = Br; Z’ = Br | A = CHF₂; Z = Br; Z’ = Br |

### Examples 692-699: 2-Heptafluoroisopropyl-4,6-Disubstituted Pyrimidines

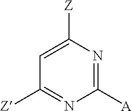

Procedure E is used to prepare the heptafluoroisopropyl derivative from the corresponding iodide.

| Ex- | | |
|---|---|---|
| am- | | |
| ple | Starting material | Product |
| 692 | A = I; Z = Cl; Z’ = Cl | A = CF(CF₃)₂; Z = Cl; Z’ = Cl |
| 693 | A = I; Z = OC₂H₅; Z’ = Cl | A = CF(CF₃)₂; Z = OC₂H₅; Z’ = Cl |
| 694 | A = I; Z = Cl; Z’ = OC₂H₅ | A = CF(CF₃)₂; Z = Cl; Z’ = OC₂H₅ |
| 695 | A = I; Z = O—Bz; Z’ = Cl | A = CF(CF₃)₂; Z = O—Bz; Z’ = Cl |
| 696 | A = I; Z = Cl; Z’ = O—Bz | A = CF(CF₃)₂; Z = Cl; Z’ = O—Bz |
| 697 | A = I; Z = Br; Z’ = Br | A = CF(CF₃)₂; Z = Br; Z’ = Br |
| 698 | A = I; Z = N(C₂H₅)₃; Z’ = Cl | A = CF(CF₃)₂; Z = N(C₂H₅)₃; Z’ = Cl |
| 699 | A = I; Z = Br; Z’ = Br | A = CF(CF₃)₂; Z = Br; Z’ = Br |

### Examples 700-709: 2-Pentafluoroethyl-6-Substituted Pyrimidines

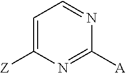

Procedure A is used to prepare the pentafluoroethyl derivative from the corresponding iodide,

| Example | Starting material | Product |
|---|---|---|
| 700 | A = I; Z = Cl | A = C₂F₃; Z = Cl |
| 701 | A = I; Z = OC₂H₅ | A = C₂F₃; Z = OC₂H₅ |
| 702 | A = I; Z = O—Bz | A = C₂F₃; Z = O—Bz |
| 703 | A = I; Z = Br | A = C₂F₃; Z = Br |
| 704 | A = I; Z = HC═O | A = C₂F₃; Z = HC═O |
| 705 | A = I; Z = CO₂CH₃ | A = C₂F₃; Z = CO₂CH₃ |
| 706 | A = I; Z = COCH₃ | A = C₂F₃; Z = COCH₃ |
| 707 | A = I; Z = CONH₂ | A = C₂F₃; Z = CONH₂ |
| 708 | A = I; Z = CN | A = C₂F₃; Z = CN |
| 709 | A = I; Z = NHCOPh | A = C₂F₃; Z = NHCOPh |

### Examples 710-719: 2-Heptafluoropropyl-6-Substituted Pyrimidines

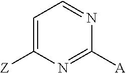

Procedure B is used to prepare the heptafluoropropyl derivative from the corresponding iodide.

| Example | Starting material | Product |
|---|---|---|
| 710 | A = I; Z = Cl | A = CF₂CF₂CF₃; Z = Cl |
| 711 | A = I; Z = OC₂H₅ | A = CF₂CF₂CF₃; Z = OC₂H₅ |
| 712 | A = I; Z = O—Bz | A = CF₂CF₂CF₃; Z = O—Bz |
| 713 | A = I; Z = Br | A = CF₂CF₂CF₃; Z = Br |
| 714 | A = I; Z = HC═O | A = CF₂CF₂CF₃; Z = HC═O |
| 715 | A = I; Z = CO₂CH₃ | A = CF₂CF₂CF₃; Z = CO₂CH₃ |
| 716 | A = I; Z = COCH₃ | A = CF₂CF₂CF₃; Z = COCH₃ |
| 717 | A = I; Z = CONH₂ | A = CF₂CF₂CF₃; Z = CONH₂ |
| 718 | A = I; Z = CN | A = CF₂CF₂CF₃; Z = CN |
| 719 | A = I; Z = NHCOPh | A = CF₂CF₂CF₃; Z = NHCOPh |

### Examples 720-729: 2-Nonafluorobutyl-6-Substituted Pyrimidines

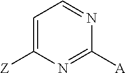

Procedure C is used to prepare the nonafluorobutyl derivative from the corresponding iodide.

| Example | Starting material | Product |
|---|---|---|
| 720 | A = I; Z = Cl | A = CF₂CF₂CF₂CF₃; Z = Cl |
| 721 | A = I; Z = OC₂H₅ | A = CF₂CF₂CF₂CF₃; Z = OC₂H₅ |
| 722 | A = I; Z = O—Bz | A = CF₂CF₂CF₂CF₃; Z = O—Bz |
| 723 | A = I; Z = Br | A = CF₂CF₂CF₂CF₃; Z = Br |
| 724 | A = I; Z = HC═O | A = CF₂CF₂CF₂CF₃; Z = HC═O |
| 725 | A = I; Z = CO₂CH₃ | A = CF₂CF₂CF₂CF₃; Z = CO₂CH₃ |
| 726 | A = I; Z = COCH₃ | A = CF₂CF₂CF₂CF₃; Z = COCH₃ |
| 727 | A = I; Z = CONH₂ | A = CF₂CF₂CF₂CF₃; Z = CONH₂ |
| 728 | A = I; Z = CN | A = CF₂CF₂CF₂CF₃; Z = CN |
| 729 | A = I; Z = NHCOPh | A = CF₂CF₂CF₂CF₃; Z = NHCOPh |

### Examples 730-739: 2-Difluoromethyl-6-Substituted Pyrimidines

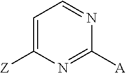

Procedure D is used to prepare the difluoromethyl derivative from the corresponding iodide.

| Example | Starting material | Product |
|---|---|---|
| 730 | A = I; Z = Cl | A = CHF₃; Z = Cl |
| 731 | A = I; Z = OC₂H₅ | A = CHF₃; Z = OC₂H₅ |
| 732 | A = I; Z = O—Bz | A = CHF₃; Z = O—Bz |
| 733 | A = I; Z = Br | A = CHF₃; Z = Br |
| 734 | A = I; Z = HC═O | A = CHF₃; Z = HC═O |
| 735 | A = I; Z = CO₂CH₃ | A = CHF₃; Z = CO₂CH₃ |
| 736 | A = I; Z = COCH₃ | A = CHF₃; Z = COCH₃ |
| 737 | A = I; Z = CONH₂ | A = CHF₃; Z = CONH₂ |
| 738 | A = I; Z = CN | A = CHF₃; Z = CN |
| 739 | A = I; Z = NHCOPh | A = CHF₃; Z = NHCOPh |

### Examples 740-749: 2-Heptafluoroisopropyl-6-Substituted Pyrimidines

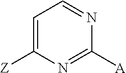

Procedure E is used to prepare the heptafluoroisopropyl derivative from the corresponding iodide.

| Example | Starting material | Product |
|---|---|---|
| 740 | A = I; Z = Cl | A = CF(CF₃)₂; Z = Cl |
| 741 | A = I; Z = OC₂H₅ | A = CF(CF₃)₂; Z = OC₂H₅ |
| 742 | A = I; Z = O—Bz | A = CF(CF₃)₂; Z = O—Bz |
| 743 | A = I; Z = Br | A = CF(CF₃)₂; Z = Br |
| 744 | A = I; Z = HC═O | A = CF(CF₃)₂; Z = HC═O |
| 745 | A = I; Z = CO₂CH₃ | A = CF(CF₃)₂; Z = CO₂CH₃ |
| 746 | A = I; Z = COCH₃ | A = CF(CF₃)₂; Z = COCH₃ |
| 747 | A = I; Z = CONH₂ | A = CF(CF₃)₂; Z = CONH₂ |
| 748 | A = I; Z = CN | A = CF(CF₃)₂; Z = CN |
| 749 | A = I; Z = NHCOPh | A = CF(CF₃)₂; Z = NHCOPh |

### Examples 750-759: 6-Pentafluoroethyl-2-Substituted Pyrimidines

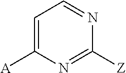

Procedure A is used to prepare the pentafluoroethyl derivative from the corresponding iodide.

| Example | Starting material | Product |
|---|---|---|
| 750 | A = I; Z = Cl | A = C₂F₃; Z = Cl |
| 751 | A = I; Z = OC₂H₅ | A = C₂F₃; Z = OC₂H₅ |
| 752 | A = I; Z = O—Bz | A = C₂F₃; Z = O—Bz |
| 753 | A = I; Z = Br | A = C₂F₃; Z = Br |
| 754 | A = I; Z = HC═O | A = C₂F₃; Z = HC═O |
| 755 | A = I; Z = CO₂CH₃ | A = C₂F₃; Z = CO₂CH₃ |
| 756 | A = I; Z = COCH₃ | A = C₂F₃; Z = COCH₃ |
| 757 | A = I; Z = CONH₂ | A = C₂F₃; Z = CONH₂ |
| 758 | A = I; Z = CN | A = C₂F₃; Z = CN |
| 759 | A = I; Z = NHCOPh | A = C₂F₃; Z = NHCOPh |

### Examples 760-769: 6-Heptafluoropropyl-2-Substituted Pyrimidines

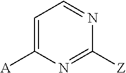

Procedure B is used to prepare the heptafluoropropyl derivative from the corresponding iodide.

| Example | Starting material | Product |
|---|---|---|
| 760 | A = I; Z = Cl | A = CF₂CF₂CF₃; Z = Cl |
| 761 | A = I; Z = OC₂H₅ | A = CF₂CF₂CF₃; Z = OC₂H₅ |
| 762 | A = I; Z = O—Bz | A = CF₂CF₂CF₃; Z = O—Bz |
| 763 | A = I; Z = Br | A = CF₂CF₂CF₃; Z = Br |
| 764 | A = I; Z = HC═O | A = CF₂CF₂CF₃; Z = HC═O |
| 765 | A = I; Z = CO₂CH₃ | A = CF₂CF₂CF₃; Z = CO₂CH₃ |
| 766 | A = I; Z = COCH₃ | A = CF₂CF₂CF₃; Z = COCH₃ |
| 767 | A = I; Z = CONH₂ | A = CF₂CF₂CF₃; Z = CONH₂ |
| 768 | A = I; Z = CN | A = CF₂CF₂CF₃; Z = CN |
| 769 | A = I; Z = NHCOPh | A = CF₂CF₂CF₃; Z = NHCOPh |

### Examples 770-779: 6-Nonafluorobutyl-2-Substituted Pyrimidines

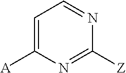

Procedure C is used to prepare the nonafluorobutyl derivative from the corresponding iodide.

| Example | Starting material | Product |
|---|---|---|
| 770 | A = I; Z = Cl | A = CF₂CF₂CF₂CF₃; Z = Cl |
| 771 | A = I; Z = OC₂H₅ | A = CF₂CF₂CF₂CF₃; Z = OC₂H₅ |
| 772 | A = I; Z = O—Bz | A = CF₂CF₂CF₂CF₃; Z = O—Bz |
| 773 | A = I; Z = Br | A = CF₂CF₂CF₂CF₃; Z = Br |
| 774 | A = I; Z = HC═O | A = CF₂CF₂CF₂CF₃; Z = HC═O |
| 775 | A = I; Z = CO₂CH₃ | A = CF₂CF₂CF₂CF₃; Z = CO₂CH₃ |
| 776 | A = I; Z = COCH₃ | A = CF₂CF₂CF₂CF₃; Z = COCH₃ |
| 777 | A = I; Z = CONH₂ | A = CF₂CF₂CF₂CF₃; Z = CONH₂ |
| 778 | A = I; Z = CN | A = CF₂CF₂CF₂CF₃; Z = CN |
| 779 | A = I; Z = NHCOPh | A = CF₂CF₂CF₂CF₃; Z = NHCOPh |

### Examples 780-789: 6-Difluoromethyl-2-Substituted Pyrimidines

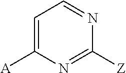

Procedure D is used to prepare the difluoromethyl derivative from the corresponding iodide.

| Example | Starting material | Product |
|---|---|---|
| 780 | A = I; Z = Cl | A = CHF₂; Z = Cl |
| 781 | A = I; Z = OC₂H₅ | A = CHF₂; Z = OC₂H₅ |
| 782 | A = I; Z = O—Bz | A = CHF₂; Z = O—Bz |
| 783 | A = I; Z = Br | A = CHF₂; Z = Br |
| 784 | A = I; Z = HC═O | A = CHF₂; Z = HC═O |
| 785 | A = I; Z = CO₂CH₃ | A = CHF₂; Z = CO₂CH₃ |
| 786 | A = I; Z = COCH₃ | A = CHF₂; Z = COCH₃ |
| 787 | A = I; Z = CONH₂ | A = CHF₂; Z = CONH₂ |
| 788 | A = I; Z = CN | A = CHF₂; Z = CN |
| 789 | A = I; Z = NHCOPh | A = CHF₂; Z = NHCOPh |

### Examples 790-799: 6-Heptafluoroisopropyl-2-Substituted Pyrimidines

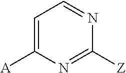

Procedure E is used to prepare the heptafluoroisopropyl derivative from the corresponding iodide.

| Example | Starting material | Product |
|---|---|---|
| 790 | A = I; Z = Cl | A = CF(CF₃)₂; Z = Cl |
| 791 | A = I; Z = OC₂H₅ | A = CF(CF₃)₂; Z = OC₂H₅ |
| 792 | A = I; Z = O—Bz | A = CF(CF₃)₂; Z = O—Bz |
| 793 | A = I; Z = Br | A = CF(CF₃)₂; Z = Br |
| 794 | A = I; Z = HC═O | A = CF(CF₃)₂; Z = HC═O |
| 795 | A = I; Z = CO₂CH₃ | A = CF(CF₃)₂; Z = CO₂CH₃ |
| 796 | A = I; Z = COCH₃ | A = CF(CF₃)₂; Z = COCH₃ |
| 797 | A = I; Z = CONH₂ | A = CF(CF₃)₂; Z = CONH₂ |
| 798 | A = I; Z = CN | A = CF(CF₃)₂; Z = CN |
| 799 | A = I; Z = NHCOPh | A = CF(CF₃)₂; Z = NHCOPh |

### Examples 808-809: 5-Pentafluoroethyl-2-Substituted Pyrimidines

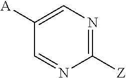

Procedure A is used to prepare the pentafluoroethyl derivative from the corresponding iodide.

| Example | Starting material | Product |
|---|---|---|
| 800 | A = I; Z = Cl | A = C₂F₃; Z = Cl |
| 801 | A = I; Z = OC₂H₅ | A = C₂F₃; Z = OC₂H₅ |
| 802 | A = I; Z = O—Bz | A = C₂F₃; Z = O—Bz |
| 803 | A = I; Z = Br | A = C₂F₃; Z = Br |
| 804 | A = I; Z = HC═O | A = C₂F₃; Z = HC═O |
| 805 | A = I; Z = CO₂CH₃ | A = C₂F₃; Z = CO₂CH₃ |
| 806 | A = I; Z = COCH₃ | A = C₂F₃; Z = COCH₃ |
| 807 | A = I; Z = CONH₂ | A = C₂F₃; Z = CONH₂ |
| 808 | A = I; Z = CN | A = C₂F₃; Z = CN |
| 809 | A = I; Z = NHCOPh | A = C₂F₃; Z = NHCOPh |

### Examples 818-819: 5-Heptafluoropropyl-2-Substituted Pyrimidines

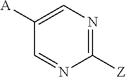

Procedure B is used to prepare the heptafluoropropyl derivative from the corresponding iodide.

| Example | Starting material | Product |
|---|---|---|
| 810 | A = I; Z = Cl | A = CF₂CF₂CF₃; Z = Cl |
| 811 | A = I; Z = OC₂H₅ | A = CF₂CF₂CF₃; Z = OC₂H₅ |
| 812 | A = I; Z = O—Bz | A = CF₂CF₂CF₃; Z = O—Bz |
| 813 | A = I; Z = Br | A = CF₂CF₂CF₃; Z = Br |
| 814 | A = I; Z = HC═O | A = CF₂CF₂CF₃Z = HC═O |
| 815 | A = I; Z = CO₂CH₃ | A = CF₂CF₂CF₃; Z = CO₂CH₃ |
| 816 | A = I; Z = COCH₃ | A = CF₂CF₂CF₃; Z = COCH₃ |
| 817 | A = I; Z = CONH₂ | A = CF₂CF₂CF₃; Z = CONH₂ |
| 818 | A = I; Z = CN | A = CF₂CF₂CF₃; Z = CN |
| 819 | A = I; Z = NHCOPh | A = CF₂CF₂CF₃; Z = NHCOPh |

### Examples 820-829: 5-Nonafluorobutyl-2-Substituted Pyrimidines

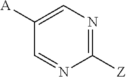

Procedure C is used to prepare the nonafluorobutyl derivative from the corresponding iodide.

| Example | Starting material | Product |
|---|---|---|
| 820 | A = I; Z = Cl | A = CF₂CF₂CF₂CF₃; Z = Cl |
| 821 | A = I; Z = OC₂H₅ | A = CF₂CF₂CF₂CF₃; Z = OC₂H₅ |
| 822 | A = I; Z = O—Bz | A = CF₂CF₂CF₂CF₃; Z = O—Bz |
| 823 | A = I; Z = Br | A = CF₂CF₂CF₂CF₃; Z = Br |
| 824 | A = I; Z = HC═O | A = CF₂CF₂CF₂CF₃; Z = HC═O |
| 825 | A = I; Z = CO₂CH₃ | A = CF₂CF₂CF₂CF₃; Z = CO₂CH₃ |
| 826 | A = I; Z = COCH₃ | A = CF₂CF₂CF₂CF₃; Z = COCH₃ |
| 827 | A = I; Z = CONH₂ | A = CF₂CF₂CF₂CF₃; Z = CONH₂ |
| 828 | A = I; Z = CN | A = CF₂CF₂CF₂CF₃; Z = CN |
| 829 | A = I; Z = NHCOPh | A = CF₂CF₂CF₂CF₃; Z = NHCOPh |

### Examples 830-839: 5-Difluoromethyl-2-Substituted Pyrimidines

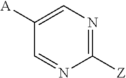

Procedure D is used to prepare the difluoromethyl derivative from the corresponding iodide.

| Example | Starting material | Product |
|---|---|---|
| 830 | A = I; Z = Cl | A = CHF₂; Z = Cl |
| 831 | A = I; Z = OC₂H₅ | A = CHF₂; Z = OC₂H₅ |
| 832 | A = I; Z = O—Bz | A = CHF₂; Z = O—Bz |
| 833 | A = I; Z = Br | A = CHF₂; Z = Br |
| 834 | A = I; Z = HC═O | A = CHF₂; Z = HC═O |
| 835 | A = I; Z = CO₂CH₃ | A = CHF₂; Z = CO₂CH₃ |
| 836 | A = I; Z = COCH₃ | A = CHF₂; Z = COCH₃ |
| 837 | A = I; Z = CONH₂ | A = CHF₂; Z = CONH₂ |
| 838 | A = I; Z = CN | A = CHF₂; Z = CN |
| 839 | A = I; Z = NHCOPh | A = CHF₂; Z = NHCOPh |

### Examples 840-849: 5-Heptafluoroisopropyl-2-Substituted Pyrimidines

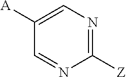

Procedure E is used to prepare the heptafluoroisopropyl derivative from the corresponding iodide.

| Example | Starting material | Product |
|---|---|---|
| 840 | A = I; Z = Cl | A = CF(CF₃)₂; Z = Cl |
| 841 | A = I; Z = OC₂H₅ | A = CF(CF₃)₂; Z = OC₂H₅ |
| 842 | A = I; Z = O—Bz | A = CF(CF₃)₂; Z = O—Bz |
| 843 | A = I; Z = Br | A = CF(CF₃)₂; Z = Br |
| 844 | A = I; Z = HC═O | A = CF(CF₃)₂; Z = HC═O |
| 845 | A = I; Z = CO₂CH₃ | A = CF(CF₃)₂; Z = CO₂CH₃ |
| 846 | A = I; Z = COCH₃ | A = CF(CF₃)₂; Z = COCH₃ |
| 847 | A = I; Z = CONH₂ | A = CF(CF₃)₂; Z = CONH₂ |
| 848 | A = I; Z = CN | A = CF(CF₃)₂; Z = CN |
| 849 | A = I; Z = NHCOPh | A = CF(CF₃)₂; Z = NHCOPh |

### Examples 850-859: 4-Pentafluoroethyl-2-Substituted Pyrimidines

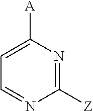

Procedure A is used to prepare the pentafluoroethyl derivative from the corresponding iodide.

| Example | Starting material | Product |
|---|---|---|
| 850 | A = I; Z = Cl | A = C₂F₃; Z = Cl |
| 851 | A = I; Z = OC₂H₅ | A = C₂F₃; Z = OC₂H₅ |
| 852 | A = I; Z = O—Bz | A = C₂F₃; Z = O—Bz |
| 853 | A = I; Z = Br | A = C₂F₃; Z = Br |
| 854 | A = I; Z = HC═O | A = C₂F₃ Z = HC═O |
| 855 | A = I; Z = CO₂CH₃ | A = C₂F₃; Z = CO₂CH₃ |
| 856 | A = I; Z = COCH₃ | A = C₂F₃; Z = COCH₃ |
| 857 | A = I; Z = CONH₂ | A = C₂F₃; Z = CONH₂ |
| 858 | A = I; Z = CN | A = C₂F₃; Z = CN |
| 859 | A = I; Z = NHCOPh | A = C₂F₃; Z = NHCOPh |

### Examples 860-869: 4-Heptafluoropropyl-2-Substituted Pyrimidines

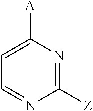

Procedure B is used to prepare the heptafluoropropyl derivative from the corresponding iodide.

| Example | Starting material | Product |
|---|---|---|
| 860 | A = I; Z = Cl | A = CF₂CF₂CF₃; Z = Cl |
| 861 | A = I; Z = OC₂H₅ | A = CF₂CF₂CF₃; Z = OC₂H₅ |
| 862 | A = I; Z = O—Bz | A = CF₂CF₂CF₃; Z = O—Bz |
| 863 | A = I; Z = Br | A = CF₂CF₂CF₃; Z = Br |
| 864 | A = I; Z = HC═O | A = CF₂CF₂CF₃Z = HC═O |
| 865 | A = I; Z = CO₂CH₃ | A = CF₂CF₂CF₃; Z = CO₂CH₃ |
| 866 | A = I; Z = COCH₃ | A = CF₂CF₂CF₃; Z = COCH₃ |
| 867 | A = I; Z = CONH₂ | A = CF₂CF₂CF₃; Z = CONH₂ |
| 868 | A = I; Z = CN | A = CF₂CF₂CF₃; Z = CN |
| 869 | A = I; Z = NHCOPh | A = CF₂CF₂CF₃; Z = NHCOPh |

### Examples 870-879: 4-Nonafluorobutyl-2-Substituted Pyrimidines

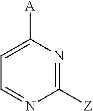

Procedure C is used to prepare the nonafluorobutyl derivative from the corresponding iodide.

| Example | Starting material | Product |
|---|---|---|
| 870 | A = I; Z = Cl | A = CF₂CF₂CF₂CF₃; Z = Cl |
| 871 | A = I; Z = OC₂H₅ | A = CF₂CF₂CF₂CF₃; Z = OC₂H₅ |
| 872 | A = I; Z = O—Bz | A = CF₂CF₂CF₂CF₃; Z = O—Bz |
| 873 | A = I; Z = Br | A = CF₂CF₂CF₂CF₃; Z = Br |
| 874 | A = I; Z = HC═O | A = CF₂CF₂CF₂CF₃; Z = HC═O |
| 875 | A = I; Z = CO₂CH₃ | A = CF₂CF₂CF₂CF₃; Z = CO₂CH₃ |
| 876 | A = I; Z = COCH₃ | A = CF₂CF₂CF₂CF₃; Z = COCH₃ |
| 877 | A = I; Z = CONH₂ | A = CF₂CF₂CF₂CF₃; Z = CONH₂ |
| 878 | A = I; Z = CN | A = CF₂CF₂CF₂CF₃; Z = CN |
| 879 | A = I; Z = NHCOPh | A = CF₂CF₂CF₂CF₃; Z = NHCOPh |

### Examples 880-889: 4-Difluoromethyl-2-Substituted Pyrimidines

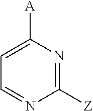

Procedure D is used to prepare the difluoromethyl derivative from the corresponding iodide.

| Example | Starting material | Product |
|---|---|---|
| 880 | A = I; Z = Cl | A = CHF₂; Z = Cl |
| 881 | A = I; Z = OC₂H₅ | A = CHF₂; Z = OC₂H₅ |
| 882 | A = I; Z = O—Bz | A = CHF₂; Z = O—Bz |
| 883 | A = I; Z = Br | A = CHF₂; Z = Br |
| 884 | A = I; Z = HC═O | A = CHF₂; Z = HC═O |
| 885 | A = I; Z = CO₂CH₃ | A = CHF₂; Z = CO₂CH₃ |
| 886 | A = I; Z = COCH₃ | A = CHF₂; Z = COCH₃ |
| 887 | A = I; Z = CONH₂ | A = CHF₂; Z = CONH₂ |
| 888 | A = I; Z = CN | A = CHF₂; Z = CN |
| 889 | A = I; Z = NHCOPh | A = CHF₂; Z = NHCOPh |

### Examples 890-899: 4-Heptafluoroisopropyl-2-Substituted Pyrimidines

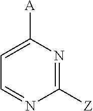

Procedure E is used to prepare the heptafluoroisopropyl derivative from the corresponding iodide.

| Example | Starting material | Product |
|---|---|---|
| 890 | A = I; Z = Cl | A = CF(CF₃)₂; Z = Cl |
| 891 | A = I; Z = OC₂H₅ | A = CF(CF₃)₂; Z = OC₂H₅ |
| 892 | A = I; Z = O—Bz | A = CF(CF₃)₂; Z = O—Bz |
| 893 | A = I; Z = Br | A = CF(CF₃)₂; Z = Br |
| 894 | A = I; Z = HC═O | A = CF(CF₃)₂; Z = HC═O |
| 895 | A = I; Z = CO₂CH₃ | A = CF(CF₃)₂; Z = CO₂CH₃ |
| 896 | A = I; Z = COCH₃ | A = CF(CF₃)₂; Z = COCH₃ |
| 897 | A = I; Z = CONH₂ | A = CF(CF₃)₂; Z = CONH₂ |
| 898 | A = I; Z = CN | A = CF(CF₃)₂; Z = CN |
| 899 | A = I; Z = NHCOPh | A = CF(CF₃)₂; Z = NHCOPh |

Pyrazines

### Examples 900-908: 2-Pentafluoroethyl-6-Substituted Pyrazines

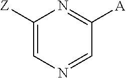

Procedure A is used to prepare the pentafluoroethyl derivatives from the corresponding iodide or bromide.

| Example | Starting material | Product |
|---|---|---|
| 900 | A = Br; Z = Cl | A = CF₂CF₃; Z = Cl |
| 901 | A = I; Z = Br | A = CF₂CF₃; Z = Br |
| 902 | A = I; Z = CO₂C₂H₅ | A = CF₂CF₃; Z = CO₂C₂H₅ |
| 903 | A = I; Z = CONH₂ | A = CF₂CF₃; Z = CONH₂ |
| 904 | A = I; Z = COCH₃ | A = CF₂CF₃; Z = COCH₃ |
| 905 | A = I; Z = CHO | A = CF₂CF₃; Z = CHO |
| 906 | A = I; Z = OBz | A = CF₂CF₃; Z = OBz |
| 907 | A = I; Z = NH—BOC | A = CF₂CF₃; Z = NH—BOC |
| 908 | A = Br; Z = CN | A = CF₂CF₃; Z = CN |

### Examples 909-917: 2-Heptafluoropropyl-6-Substituted Pyrazines

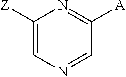

Procedure B is used to prepare the heptafluoropropyl derivative from the corresponding iodide or bromide.

| Example | Starting material | Product |
|---|---|---|
| 909 | A = Br; Z = Cl | A = CF₂CF₂CF₃; Z = Cl |
| 910 | A = I; Z = Br | A = CF₂CF₂CF₃; Z = Br |
| 911 | A = I; Z = CO₂C₂H₅ | A = CF₂CF₂CF₃; Z = CO₂C₂H₅ |
| 912 | A = I; Z = CONH₂ | A = CF₂CF₂CF₃; Z = CONH₂ |
| 913 | A = I; Z = COCH₃ | A = CF₂CF₂CF₃; Z = COCH₃ |
| 914 | A = I; Z = CHO | A = CF₂CF₂CF₃; Z = CHO |
| 915 | A = I; Z = OBz | A = CF₂CF₂CF₃; Z = OBz |
| 916 | A = I; Z = NH—BOC | A = CF₂CF₂CF₃; Z = NH—BOC |
| 917 | A = Br; Z = CN | A = CF₂CF₂CF₃; Z = CN |

### Examples 918-926: 2-Nonafluorobutyl-6-Substituted Pyrazines

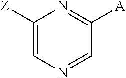

Procedure C is used to prepare the nonafluorobutyl derivative from the corresponding iodide or bromide.

| Example | Starting material | Product |
|---|---|---|
| 918 | A = Br; Z = Cl | A = CF₂CF₂CF₂CF₃; Z = Cl |
| 919 | A = I; Z = Br | A = CF₂CF₂CF₂CF₃; Z = Br |
| 920 | A = I; Z = CO₂C₂H₅ | A = CF₂CF₂CF₂CF₃; Z = CO₂C₂H₅ |
| 921 | A = I; Z = CONH₂ | A = CF₂CF₂CF₂CF₃; Z = CONH₂ |
| 922 | A = I; Z = COCH₃ | A = CF₂CF₂CF₂CF₃; Z = COCH₃ |
| 923 | A = I; Z = CHO | A = CF₂CF₂CF₂CF₃; Z = CHO |
| 924 | A = I; Z = OBz | A = CF₂CF₂CF₂CF₃; Z = OBz |
| 925 | A = I; Z = NH—BOC | A = CF₂CF₂CF₂CF₃; Z = NH—BOC |
| 926 | A = Br; Z = CN | A = CF₂CF₂CF₂CF₃; Z = CN |

### Examples 927-935: 2-Difluoromethyl-6-Substituted Pyrazines

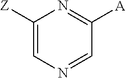

Procedure D is used to prepare the difluoromethyl derivative from the corresponding iodide or bromide.

| Example | Starting material | Product |
|---|---|---|
| 927 | A = Br; Z = Cl | A = CHF₂; Z = Cl |
| 928 | A = I; Z = Br | A = CHF₂; Z = Br |
| 929 | A = I; Z = CO₂C₂H₅ | A = CHF₂; Z = CO₂C₂H₅ |
| 930 | A = I; Z = CONH₂ | A = CHF₂; Z = CONH₂ |
| 931 | A = I; Z = COCH₃ | A = CHF₂; Z = COCH₃ |
| 932 | A = I; Z = CHO | A = CHF₂; Z = CHO |
| 933 | A = I; Z = OBz | A = CHF₂; Z = OBz |
| 934 | A = I; Z = NH—BOC | A = CHF₂; Z = NH—BOC |
| 935 | A = Br; Z = CN | A = CHF₂; Z = CN |

### Examples 936-944: 2-Heptafluoroisopropyl-6-Substituted Pyrazines

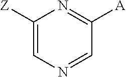

Procedure E is used to prepare the heptafluoroisopropyl derivative from the corresponding iodide or bromide.

| Example | Starting material | Product |
|---|---|---|
| 936 | A = Br; Z = Cl | A = CF₂(CF₃)₂; Z = Cl |
| 937 | A = I; Z = Br | A = CF₂(CF₃)₂ = Br |
| 938 | A = I; Z = CO₂C₂H₅ | A = CF₂(CF₃)₂ = CO₂C₂H₅ |
| 939 | A = I; Z = CONH₂ | A = CF₂(CF₃)₂ = CONH₂ |
| 940 | A = I; Z = COCH₃ | A = CF₂(CF₃)₂ = COCH₃ |
| 941 | A = I; Z = CHO | A = CF₂(CF₃)₂ = CHO |
| 942 | A = I; Z = OBz | A = CF₂(CF₃)₂ = OBz |
| 943 | A = I; Z = NH—BOC | A = CF₂(CF₃)₂ = NH—BOC |
| 944 | A = Br; Z = CN | A = CF₂(CF₃)₂ = CN |

### Examples 945-953: 2-Pentafluoroethyl-5-Substituted Pyrazines

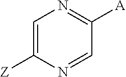

Procedure A is used to prepare the pentafluoroethyl derivative from the corresponding bromide.

| Example | Starting material | Product |
|---|---|---|
| 945 | A = Br; Z = Cl | A = CF₂CF₃; Z = Cl |
| 946 | A = I; Z = Br | A = CF₂CF₃; Z = Br |
| 947 | A = I; Z = CO₂C₂H₅ | A = CF₂CF₃; Z = CO₂C₂H₅ |
| 948 | A = I; Z = CONH₂ | A = CF₂CF₃; Z = CONH₂ |
| 949 | A = I; Z = COCH₃ | A = CF₂CF₃; Z = COCH₃ |
| 950 | A = I; Z = CHO | A = CF₂CF₃; Z = CHO |
| 951 | A = I; Z = OBz | A = CF₂CF₃; Z = OBz |
| 952 | A = I; Z = NH—BOC | A = CF₂CF₃; Z = NH—BOC |
| 953 | A = Br; Z = CN | A = CF₂CF₃; Z = CN |

### Examples 954-962: 2-Heptafluoropropyl-5-Substituted Pyrazines

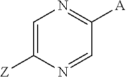

Procedure B is used to prepare the heptafluoropropyl derivative from the corresponding iodide or bromide.

| Example | Starting material | Product |
|---|---|---|
| 954 | A = Br; Z = Cl | A = CF₂CF₂CF₃; Z = Cl |
| 955 | A = I; Z = Br | A = CF₂CF₂CF₃; Z = Br |
| 956 | A = I; Z = CO₂C₂H₅ | A = CF₂CF₂CF₃; Z = CO₂C₂H₅ |
| 957 | A = I; Z = CONH₂ | A = CF₂CF₂CF₃; Z = CONH₂ |
| 958 | A = I; Z = COCH₃ | A = CF₂CF₂CF₃; Z = COCH₃ |
| 959 | A = I; Z = CHO | A = CF₂CF₂CF₃; Z = CHO |
| 960 | A = I; Z = OBz | A = CF₂CF₂CF₃; Z = OBz |
| 961 | A = I; Z = NH—BOC | A = CF₂CF₂CF₃; Z = NH—BOC |
| 962 | A = Br; Z = CN | A = CF₂CF₂CF₃; Z = CN |

### Examples 963-971: 2-Nonafluorobutyl-5-Substituted Pyrazines

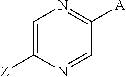

Procedure C is used to prepare the nonafluorobutyl derivative from the corresponding iodide or bromide.

| Example | Starting material | Product |
|---|---|---|
| 963 | A = Br; Z = Cl | A = CF₂CF₂CF₂CF₃; Z = Cl |
| 964 | A = I; Z = Br | A = CF₂CF₂CF₂CF₃; Z = Br |
| 965 | A = I; Z = CO₂C₂H₅ | A = CF₂CF₂CF₂CF₃; Z = CO₂C₂H₅ |
| 966 | A = I; Z = CONH₂ | A = CF₂CF₂CF₂CF₃; Z = CONH₂ |
| 967 | A = I; Z = COCH₃ | A = CF₂CF₂CF₂CF₃; Z = COCH₃ |
| 968 | A = I; Z = CHO | A = CF₂CF₂CF₂CF₃; Z = CHO |
| 969 | A = I; Z = OBz | A = CF₂CF₂CF₂CF₃; Z = OBz |
| 970 | A = I; Z = NH—BOC | A = CF₂CF₂CF₂CF₃; Z = NH—BOC |
| 971 | A = Br; Z = CN | A = CF₂CF₂CF₂CF₃; Z = CN |

### Examples 972-980: 2-Difluoromethyl-5-Substituted Pyrazines

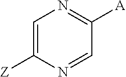

Procedure D is used to prepare the difluoromethyl derivative from the corresponding iodide or bromide.

| Example | Starting material | Product |
|---|---|---|
| 972 | A = Br; Z = Cl | A = CHF₂; Z = Cl |
| 973 | A = I; Z = Br | A = CHF₂; Z = Br |
| 974 | A = I; Z = CO₂C₂H₅ | A = CHF₂; Z = CO₂C₂H₅ |
| 975 | A = I; Z = CONH₂ | A = CHF₂; Z = CONH₂ |
| 976 | A = I; Z = COCH₃ | A = CHF₂; Z = COCH₃ |
| 977 | A = I; Z = CHO | A = CHF₂; Z = CHO |
| 978 | A = I; Z = OBz | A = CHF₂; Z = OBz |
| 979 | A = I; Z = NH—BOC | A = CHF₂; Z = NH—BOC |
| 980 | A = Br; Z = CN | A = CHF₂; Z = CN |

### Examples 981-989: 2-Heptafluoroisopropyl-5-Substituted Pyrazines

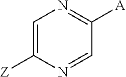

Procedure E is used to prepare the heptafluoroisopropyl derivative from the corresponding iodide or bromide.

| Example | Starting material | Product |
|---|---|---|
| 981 | A = Br; Z = Cl | A = CF(CF₃)₂; Z = Cl |
| 982 | A = I; Z = Br | A = CF(CF₃)₂ = Br |
| 983 | A = I; Z = CO₂C₂H₅ | A = CF(CF₃)₂ = CO₂C₂H₅ |
| 984 | A = I; Z = CONH₂ | A = CF(CF₃)₂ = CONH₂ |
| 985 | A = I; Z = COCH₃ | A = CF(CF₃)₂ = COCH₃ |
| 986 | A = I; Z = CHO | A = CF(CF₃)₂ = CHO |
| 987 | A = I; Z = OBz | A = CF(CF₃)₂ = OBz |
| 988 | A = I; Z = NH—BOC | A = CF(CF₃)₂ = NH—BOC |
| 989 | A = Br; Z = CN | A = CF(CF₃)₂ = CN |

### Examples 990-998: 2-Pentafluorobutyl-3-Substituted Pyrazines

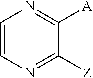

Procedure A is used to prepare the pentafluoroethyl derivative from the corresponding bromide.

| Example | Starting material | Product |
|---|---|---|
| 990 | A = Br; Z = Cl | A = CF₂CF₃; Z = Cl |
| 991 | A = I; Z = Br | A = CF₂CF₃; Z = Br |
| 992 | A = I; Z = CO₂C₂H₅ | A = CF₂CF₃; Z = CO₂C₂H₅ |
| 993 | A = I; Z = CONH₂ | A = CF₂CF₃; Z = CONH₂ |
| 994 | A = I; Z = COCH₃ | A = CF₂CF₃; Z = COCH₃ |
| 995 | A = I; Z = CHO | A = CF₂CF₃; Z = CHO |
| 996 | A = I; Z = OBz | A = CF₂CF₃; Z = OBz |
| 997 | A = I; Z = NH—BOC | A = CF₂CF₃; Z = NH—BOC |
| 998 | A = Br; Z = CN | A = CF₂CF₃; Z = CN |

### Examples 999-1007: 2-Heptafluoropropyl-3-Substituted Pyrazines

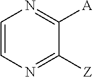

Procedure B is used to prepare the heptafluoropropyl derivative from the corresponding iodide or bromide.

| Example | Starting material | Product |
|---|---|---|
| 999 | A = Br; Z = Cl | A = CF₂CF₂CF₃; Z = Cl |
| 1000 | A = I; Z = Br | A = CF₂CF₂CF₃; Z = Br |
| 1001 | A = I; Z = CO₂C₂H₅ | A = CF₂CF₂CF₃; Z = CO₂C₂H₅ |
| 1002 | A = I; Z = CONH₂ | A = CF₂CF₂CF₃; Z = CONH₂ |
| 1003 | A = I; Z = COCH₃ | A = CF₂CF₂CF₃; Z = COCH₃ |
| 1004 | A = I; Z = CHO | A = CF₂CF₂CF₃; Z = CHO |
| 1005 | A = I; Z = OBz | A = CF₂CF₂CF₃; Z = OBz |
| 1006 | A = I; Z = NH—BOC | A = CF₂CF₂CF₃; Z = NH—BOC |
| 1007 | A = Br; Z = CN | A = CF₂CF₂CF₃; Z = CN |

### Examples 1008-1016: 2-Nonafluorobutyl-3-Substituted Pyrazines

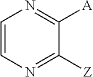

Procedure C is used to prepare the nonafluorobutyl derivative from the corresponding iodide or bromide.

| Example | Starting material | Product |
|---|---|---|
| 1008 | A = Br; Z = Cl | A = CF₂CF₂CF₂CF₃; Z = Cl |
| 1009 | A = I; Z = Br | A = CF₂CF₂CF₂CF₃; Z = Br |
| 1010 | A = I; Z = CO₂C₂H₅ | A = CF₂CF₂CF₂CF₃; Z = CO₂C₂H₅ |
| 1011 | A = I; Z = CONH₂ | A = CF₂CF₂CF₂CF₃; Z = CONH₂ |
| 1012 | A = I; Z = COCH₃ | A = CF₂CF₂CF₂CF₃; Z = COCH₃ |
| 1013 | A = I; Z = CHO | A = CF₂CF₂CF₂CF₃; Z = CHO |
| 1014 | A = I; Z = OBz | A = CF₂CF₂CF₂CF₃; Z = OBz |
| 1015 | A = I; Z = NH—BOC | A = CF₂CF₂CF₂CF₃; Z = NH—BOC |
| 1016 | A = Br; Z = CN | A = CF₂CF₂CF₂CF₃; Z = CN |

### Examples 1017-1025: 2-Difluoromethyl-3,5-Substituted Pyrazines

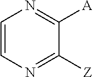

Procedure D is used to prepare the difluoromethyl derivative from the corresponding iodide or bromide.

| Example | Starting material | Product |
|---|---|---|
| 1017 | A = Br; Z = Cl | A = CHF₂; Z = Cl |
| 1018 | A = I; Z = Br | A = CHF₂; Z = Br |
| 1019 | A = I; Z = CO₂C₂H₅ | A = CHF₂; Z = CO₂C₂H₅ |
| 1020 | A = I; Z = CONH₂ | A = CHF₂; Z = CONH₂ |
| 1021 | A = I; Z = COCH₃ | A = CHF₂; Z = COCH₃ |
| 1022 | A = I; Z = CHO | A = CHF₂; Z = CHO |
| 1023 | A = I; Z = OBz | A = CHF₂; Z = OBz |
| 1024 | A = I; Z = NH—BOC | A = CHF₂; Z = NH—BOC |
| 1025 | A = Br; Z = CN | A = CHF₂; Z = CN |

### Examples 1026-1034: 2-Heptafluoroisopropyl-3,5-Substituted Pyrazines

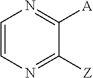

Procedure E is used to prepare the heptafluoroisopropyl derivative from the corresponding iodide or bromide.

| Example | Starting material | Product |
|---|---|---|
| 1026 | A = Br; Z = Cl | A = CF(CF₃)₂; Z = Cl |
| 1027 | A = I; Z = Br | A = CF(CF₃)₂ = Br |
| 1028 | A = I; Z = CO₂C₂H₅ | A = CF(CF₃)₂ = CO₂C₂H₅ |
| 1029 | A = I; Z = CONH₂ | A = CF(CF₃)₂ = CONH₂ |
| 1030 | A = I; Z = COCH₃ | A = CF(CF₃)₂ = COCH₃ |
| 1031 | A = I; Z = CHO | A = CF(CF₃)₂ = CHO |
| 1032 | A = I; Z = OBz | A = CF(CF₃)₂ = OBz |
| 1033 | A = I; Z = NH—BOC | A = CF(CF₃)₂ = NH—BOC |
| 1034 | A = Br; Z = CN | A = CF(CF₃)₂; Z = CN |

### Examples 1035-1044: 2-Pentafluoroethyl-3,5-Disubstituted Pyrazines

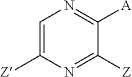

Procedure A is used to prepare the pentafluoroethyl derivative from the corresponding iodide or bromide.

| Example | Starting material | Product |
|---|---|---|
| 1035 | A = I; Z = Cl; Z′ = Cl | A = CF₂CF₃; Z = Cl; Z′ = Cl |
| 1036 | A = Br; Z = Cl; Z′ = Cl | A = CF₂CF₃; Z = Cl; Z′ = Cl |
| 1037 | A = I; Z = Br; Z′ = Br | A = CF₂CF₃; Z = Br; Z′ = Br |
| 1038 | A = I; Z = CO₂C₂H₅; Z′ = Cl | A = CF₂CF₃; Z = CO₂C₂H₅; Z′ = Cl |
| 1039 | A = I; Z = Cl; Z′ = CO₂C₂H₅ | A = CF₂CF₃; Z = Cl; Z′ = CO₂C₂H₅ |
| 1040 | A = I; Z = O—Bz; Z′ = Cl | A = CF₂CF₃; Z = O—Bz; Z′ = Cl |
| 1041 | A = I; Z = Cl; Z′ = O—Bz | A = CF₂CF₃; Z = Cl; Z′ = O—Bz |
| 1042 | A = I; Z = Br; Z′ = | A = CF₂CF₃; Z = Br; Z′ = |
| | NH—BOC | NH—BOC |
| 1043 | A = I; Z = Cl; Z′ = | A = CF₂CF₃; Z = Cl; Z′ = |
| | NH—BOC | NH—BOC |
| 1044 | A = I; Z = NH—BOC; | A = CF₂CF₃; Z = NH—BOC; |
| | Z′ = Br | Z′ = Br |

### Examples 1045-1054: 2-Heptafluoropropyl-3,5-Disubstituted Pyrazines

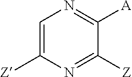

Procedure B is used to prepare the heptafluoropropyl derivative from the corresponding iodide or bromide.

| Example | Starting material | Product |
|---|---|---|
| 1045 | A = I; Z = Cl; Z′ = Cl | A = CF₂CF₂CF₃; Z = Cl; Z′ = Cl |
| 1046 | A = Br; Z = Cl; Z′ = Cl | A = CF₂CF₃; Z = Cl, Z′ = Cl |
| 1047 | A = I; Z = Br; Z′ = Br | A = CF₂CF₂CF₃; Z = Br; Z′ = Br |
| 1048 | A = I; Z = CO₂C₂H₅; | A = CF₂CF₂CF₃; Z = CO₂C₂H₅; |
| | Z′ = Cl | Z′ = Cl |
| 1049 | A = I; Z = Cl; Z′ = | A = CF₂CF₂CF₃; Z = Cl; Z′ = |
| | CO₂C₂H₅ | CO₂C₂H₅ |
| 1050 | A = I; Z = O—Bz; Z′ = Cl | A = CF₂CF₂CF₃; Z = O—Bz; Z′ = Cl |
| 1051 | A = I; Z = Cl; Z′ = O—Bz | A = CF₂CF₂CF₃; Z = Cl; Z′ = O—Bz |
| 1052 | A = I; Z = Br; Z′ = | A = CF₂CF₂CF₃; Z = Br; Z′ = |
| | NH—BOC | NH—BOC |
| 1053 | A = I; Z = Cl; Z′ = | A = CF₂CF₂CF₃; Z = Cl; Z′ = |
| | NH—BOC | NH—BOC |
| 1054 | A = I; Z = NH—BOC; | A = CF₂CF₂CF₃; Z = NH—BOC; |
| | Z′ = Br | Z′ = Br |

### Examples 1055-1064: 2-Nonafluorobutyl-3,5-Disubstituted Pyrazines

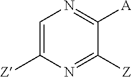

Procedure C is used to prepare the nonafluorobutyl derivative from the corresponding iodide or bromide.

| Example | Starting material | Product |
|---|---|---|
| 1055 | A = I; Z = Cl; Z′ = Cl | A = CF₂CF₂CF₂CF₃; Z = Cl; Z′ = Cl |
| 1056 | A = Br; Z = Cl; Z′ = Cl | A = CF₂CF₃; Z = Cl; Z′ = Cl |
| 1057 | A = I; Z = Br; Z′ = Br | A = CF₂CF₂CF₂CF₃; Z = Br; Z′ = Br |
| 1058 | A = I; Z = CO₂C₂H₅; | A = CF₂CF₂CF₂CF₃; Z = CO₂C₂H₅; |
| | Z′ = Cl | Z′ = Cl |
| 1059 | A = I; Z = Cl; Z′ = | A = CF₂CF₂CF₂CF₃; Z = Cl; Z′ = |
| | CO₂C₂H₅ | CO₂C₂H₅ |
| 1060 | A = I; Z = O—Bz; | A = CF₂CF₂CF₂CF₃; Z = O—Bz; |
| | Z′ = Cl | Z′ = Cl |
| 1061 | A = I; Z = Cl; Z′ = | A = CF₂CF₂CF₂CF₃; Z = Cl; Z′ = |
| | O—Bz | O—BZ |
| 1062 | A = I; Z = Br; Z′ = | A = CF₂CF₂CF₂CF₃; Z = Br; Z′ = |
| | NH—BOC | NH—BOC |
| 1063 | A = I; Z = Cl; Z′ = | A = CF₂CF₂CF₂CF₃; Z = Cl; Z′ = |
| | NH—BOC | NH—BOC |
| 1064 | A = I; Z = NH—BOC; | A = CF₂CF₂CF₂CF₃; Z = NH—BOC; |
| | Z′ = Br | Z′ = Br |

### Examples 1065-1074: 2-Difluoromethyl-3,5-Disubstituted Pyrazines

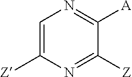

Procedure D is used to prepare the difluoromethyl derivative from the corresponding iodide or bromide.

| Example | Starting material | Product |
|---|---|---|
| 1065 | A = I; Z = Cl; Z′ = Cl | A = CHF₂; Z = Cl; Z′ = Cl |
| 1066 | A = Br; Z = Cl; Z′ = Cl | A = CF₂CF₃; Z = Cl; Z′ = Cl |
| 1067 | A = I; Z = Br; Z′ = Br | A = CHF₂; Z = Br; Z′ = Br |
| 1068 | A = I; Z = CO₂C₂H₅; Z′ = Cl | A = CHF₂; Z = CO₂C₂H₅; Z′ = Cl |
| 1069 | A = I; Z = Cl; Z′ = CO₂C₂H₅ | A = CHF₂; Z = Cl; Z′ = CO₂C₂H₅ |
| 1070 | A = I; Z = O—Bz; Z′ = Cl | A = CHF₂; Z = O—Bz; Z′ = Cl |
| 1071 | A = I; Z = Cl; Z′ = O—Bz | A = CHF₂; Z = Cl; Z′ = O—Bz |
| 1072 | A = I; Z = Br; Z′ = | A = CHF₂; Z = Br; Z′ = |
| | NH—BOC | NH—BOC |
| 1073 | A = I; Z = Cl; Z′ = | A = CHF₂; Z = Cl; Z′ = |
| | NH—BOC | NH—BOC |
| 1074 | A = I; Z = NH—BOC; | A = CHF₂; Z = NH—BOC; |
| | Z′ = Br | Z′ = Br |

### Examples 1075-1084: 2-Heptafluoroisopropyl-3-Disubstituted Pyrazines

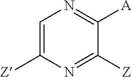

Procedure E is used to prepare the heptafluoroisopropyl derivative from the corresponding iodide or bromide.

| Example | Starting material | Product |
|---|---|---|
| 1075 | A = I; Z = Cl; Z′ = Cl | A = CF(CF₃)₂; Z = Cl; Z′ = Cl |
| 1076 | A = Br; Z = Cl; Z′ = Cl | A = CF₂CF₃; Z = Cl; Z′ = Cl |
| 1077 | A = I; Z = Br; Z′ = Br | A = CF(CF₃)₂; Z = Br; Z′ = Br |
| 1078 | A = I; Z = CO₂C₂H₅; | A = CF(CF₃)₂; Z = CO₂C₂H₅; |
| | Z′ = Cl | Z′ = Cl |
| 1079 | A = I; Z = Cl; Z′ = | A = CF(CF₃)₂; Z = Cl; Z′ = |
| | CO₂C₂H₅ | CO₂C₂H₅ |
| 1080 | A = I; Z = O—Bz; Z′ = Cl | A = CF(CF₃)₂; Z = O—Bz; Z′ = Cl |
| 1081 | A = I; Z = Cl; Z′ = O—Bz | A = CF(CF₃)₂; Z = Cl; Z′ = O—Bz |
| 1082 | A = I; Z = Br; Z′ = | A = CF(CF₃)₂; Z = Br; Z′ = |
| | NH—BOC | NH—BOC |
| 1083 | A = I; Z = Cl; Z′ = | A = CF(CF₃)₂; Z = Cl; Z′ = |
| | NH—BOC | NH—BOC |
| 1084 | A = I; Z = NH—BOC; | A = CF(CF₃)₂; Z = NH—BOC; |
| | Z′ = Br | Z′ = Br |

### Examples 1085-1094: 2-Pentafluoroethyl-3,6-Disubstituted Pyrazines

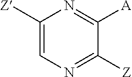

Procedure A is used to prepare the pentafluoroethyl derivative from the corresponding iodide or bromide.

| Example | Starting material | Product |
|---|---|---|
| 1085 | A = I; Z = Cl; Z′ = Cl | A = CF₂CF₃; Z = Cl; Z′ = Cl |
| 1086 | A = Br; Z = Cl; Z′ = Cl | A = CF₂CF₃; Z = Cl; Z′ = Cl |
| 1087 | A = I; Z = Br; Z′ = Br | A = CF₂CF₃; Z = Br; Z′ = Br |
| 1088 | A = I; Z = CO₂C₂H₅; Z′ = Cl | A = CF₂CF₃; Z = CO₂C₂H₅; Z′ = Cl |
| 1089 | A = I; Z = Cl; Z′ = CO₂C₂H₅ | A = CF₂CF₃; Z = Cl; Z′ = CO₂C₂H₅ |
| 1090 | A = I; Z = O—Bz; Z′ = Cl | A = CF₂CF₃; Z = O—Bz; Z′ = Cl |
| 1091 | A = I; Z = Cl; O—Bz | A = CF₂CF₃; Z = Cl; Z′ = O—Bz |
| 1092 | A = I; Z = Br; Z′ = | A = CF₂CF₃; Z = Br; Z′ = |
| | NH—BOC | NH—BOC |
| 1093 | A = I; Z = Cl; Z′ = | A = CF₂CF₃; Z = Cl; Z′ = |
| | NH—BOC | NH—BOC |
| 1094 | A = I; Z = NH—BOC; | A = CF₂CF₃; Z = NH—BOC; |
| | Z′ = Br | Z′ = Br |

### Examples 1095-1104: 2-Heptafluoropropyl-3,6-Disubstituted Pyrazines

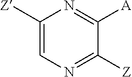

Procedure B is used to prepare the heptafluoropropyl derivative from the corresponding iodide or bromide.

| Example | Starting material | Product |
|---|---|---|
| 1095 | A = I; Z = Cl; Z′ = Cl | A = CF₂CF₂CF₃; Z = Cl; Z′ = Cl |
| 1096 | A = Br; Z = Cl; Z′ = Cl | A = CF₂CF₃; Z = Cl; Z′ = Cl |
| 1097 | A = I; Z = Br; Z′ = Br | A = CF₂CF₂CF₃; Z = Br; Z′ = Br |
| 1098 | A = I; Z = CO₂C₂H₅; | A = CF₂CF₂CF₃; Z = CO₂C₂H₅; |
| | Z′ = Cl | Z′ = Cl |
| 1099 | A = I; Z = Cl; Z′ = | A = CF₂CF₂CF₃; Z = Cl; Z′ = |
| | CO₂C₂H₅ | CO₂C₂H₅ |
| 1100 | A = I; Z = O—Bz; Z′ = Cl | A = CF₂CF₂CF₃; Z = O—Bz; Z′ = Cl |
| 1101 | A = I; Z = Cl; Z′ = O—Bz | A = CF₂CF₂CF₃; Z = Cl; Z′ = O—Bz |
| 1102 | A = I; Z = Br; Z′ = | A = CF₂CF₂CF₃; Z = Br; Z′ = |
| | NH—BOC | NH—BOC |
| 1103 | A = I; Z = Cl; Z′ = | A = CF₂CF₂CF₃; Z = Cl; Z′ = |
| | NH—BOC | NH—BOC |
| 1104 | A = I; Z = NH—BOC; | A = CF₂CF₂CF₃; Z = NH—BOC; |
| | Z′ = Br | Z′ = Br |

### Examples 1105-1114: 2-Nonafluorobutyl-3,6-Disubstituted Pyrazines

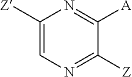

Procedure C is used to prepare the nonafluorobutyl derivative from the corresponding iodide or bromide.

| Example | Starting material | Product |
|---|---|---|
| 1105 | A = I; Z = Cl; Z′ = Cl | A = CF₂CF₂CF₂CF₃; Z = Cl; Z′ = Cl |
| 1106 | A = Br; Z = Cl; Z′ = Cl | A = CF₂CF3; Z = Cl; Z′ = Cl |
| 1107 | A = I; Z = Br; Z′ = Br | A = CF₂CF₂CF₂CF₃; Z = Br; Z′ = Br |
| 1108 | A = I; Z = CO₂C₂H₅; | A = CF₂CF₂CF₂CF₃; Z = CO₂C₂H₅; |
| | Z′ = Cl | Z′ = Cl |
| 1109 | A = I; Z = Cl; Z′ = | A = CF₂CF₂CF₂CF₃; Z = Cl; Z′ = |
| | CO₂C₂H₅ | CO₂C₂H₅ |
| 1110 | A = I; Z = O—Bz; | A = CF₂CF₂CF₂CF₃; Z = O—Bz; |
| | Z′ = Cl | Z′ = Cl |
| 1111 | A = I; Z = Cl; Z′ = | A = CF₂CF₂CF₂CF₃; Z = Cl; Z′ = |
| | O—Bz | O—Bz |
| 1112 | A = I; Z = Br; Z′ = | A = CF₂CF₂CF₂CF₃; Z = Br; Z′ = |
| | NH—BOC | NH—BOC |
| 1113 | A = I; Z = Cl; Z′ = | A = CF₂CF₂CF₂CF₃; Z = Cl; |
| | NH—BOC | Z′ = NH—BOC |
| 1114 | A = I; Z = NH—BOC; | A = CF₂CF₂CF₂CF₃; Z = NH—BOC; |
| | Z′ = Br | Z′ = Br |

### Examples 1115-1124: 2-Difluoromethyl-3,6-Disubstituted Pyrazines

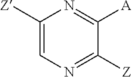

Procedure D is used to prepare the difluoromethyl derivative from the corresponding iodide or bromide.

| Example | Starting material | Product |
|---|---|---|
| 1115 | A = I; Z = Cl; Z′ = Cl | A = CF₂CF₂CF₂CF₃; Z = Cl; |
| | | Z′ = Cl |
| 1116 | A = Br; Z = Cl; Z′ = Cl | A = CF₂CF₃; Z = Cl; Z′ =Cl |
| 1117 | A = I; Z = Br; Z′ = Br | A = CHF₂; Z = Br; Z′ = Br |
| 1118 | A = I; Z = CO₂C₂H₅; Z′ = Cl | A = CHF₂; Z = CO₂C₂H₅; Z′ = Cl |
| 1119 | A = I; Z = Cl; Z′ = CO₂C₂H₅ | A = CHF₂; Z = Cl; Z′ = CO₂C₂H₅ |
| 1120 | A = I; Z = O—Bz; Z′ = Cl | A = CHF₂; Z = O—Bz; Z′ = Cl |
| 1121 | A = I; Z = Cl; Z′ = O—Bz | A = CHF₂; Z = Cl; Z′ = O—Bz |
| 1122 | A = I; Z = Br, Z′ = | A = CHF₂; Z = Br; Z′ = |
| | NH—BOC | NH—BOC |
| 1123 | A = I; Z = Cl; Z′ = | A = CHF₂; Z = Cl; Z′ = |
| | NH—BOC | NH—BOC |
| 1124 | A = I; Z = NH—BOC; | A = CHF₂; Z = NH—BOC; |
| | Z′ = Br | Z′ = Br |

### Examples 1125-1134: 2-Heptafluoroisopropyl-3,6-Disubstituted Pyrazines

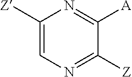

Procedure E is used to prepare the heptafluoroisopropyl derivative from the corresponding iodide or bromide.

| Example | Starting material | Product |
|---|---|---|
| 1125 | A = I; Z = Cl; Z′ = Cl | A = CF(CF₃)₂; Z = Cl; Z′ = Cl |
| 1126 | A = I; Z = Cl; Z′ = Cl | A = CF₂CF₃; Z = Cl; Z′ = Cl |
| 1127 | A = I; Z = Br; Z′ =Br | A = CF(CF₃)₂; Z = Br; Z′ = Br |
| 1128. | A = I; Z = CO₂C₂H₅; | A = CF(CF₃)₂; Z = CO₂C₂H₅; |
| | Z′ = Cl | Z′ = Cl |
| 1129 | A = I; Z = Cl; Z′ = | A = CF(CF₃)₂; Z = Cl; Z′ = |
| | CO₂C₂H₅ | CO₂C₂H₅ |
| 1130 | A = I; Z = O—Bz; Z′ = Cl | A = CF(CF₃)₂; Z = O—Bz: Z′ = Cl |
| 1131 | A = I; Z = Cl; Z′ = O—Bz | A = CF(CF₃)₂; Z = Cl; Z′ = O—Bz |
| 1132 | A = I; Z = Br; Z′ = | A = CF(CF₃)₂; Z = Br; Z′ = |
| | NH—BOC | NH—BOC |
| 1133 | A = I; Z = Cl; Z′ = | A = CF(CF₃)₂; Z = Cl; Z′ = |
| | NH—BOC | NH—BOC |
| 1134 | A = I; Z = NH—BOC; | A = CF(CF₃)₂; Z = NH—BOC; |
| | Z′ = Br | Z′ = Br |

### Examples 1135-1144: 2-Pentafluoroethyl-5,6-Disubstituted Pyrazines

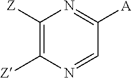

Procedure A is used to prepare the pentafluoroethyl derivative from the corresponding iodide or bromide.

| Example | Starting material | Product |
|---|---|---|
| 1135 | A = I: Z = Cl; Z′ = Cl | A = CF₂CF₃; Z = Cl; Z′ = Cl |
| 1136 | A = Br; Z = Cl; Z′ Cl | A = CF₂CF₃; Z = Cl; Z′ = Cl |
| 1137 | A = I; Z = Br; Z′ = Br | A = CF₂CF₃; Z = Br; Z′ = Br |
| 1138 | A = I; Z = CO₂C₂H₅; Z′ = Cl | A = CF₂CF₃; Z = CO₂C₂H₅; Z′ = Cl |
| 1139 | A = I; Z = Cl; Z′ = CO₂C₂H₅ | A = CF₂CF₃; Z = Cl; Z′ = CO₂C₂H₅ |
| 1140 | A = I; Z = O—Bz; Z′ = Cl | A = CF₂CF₃; Z = O—Bz; Z′ = Cl |
| 1141 | A = I; Z = Cl; Z′ = O—Bz | A = CF₂CF₃; Z = Cl; Z′ = O—Bz |
| 1142 | A = I; Z = Br; Z′ = | A = CF₂CF₃; Z = Br; Z′ = |
| | NH—BOC | NH—BOC |
| 1143 | A = I; Z = Cl; Z′ = | A = CF₂CF₃; Z = Cl: Z′ = |
| | NH—BOC | NH—BOC |
| 1144 | A = I; Z = NH—BOC; | A = CF₂CF₃; Z = NH—BOC; |
| | Z′ = Br | Z′ = Br |

### Examples 1145-1154: 2-Heptafluoropropyl-5,6-Disubstituted Pyrazines

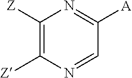

Procedure B is used to prepare the heptafluoropropyl derivative from the corresponding iodide or bromide.

| Example | Starting Material | Product |
|---|---|---|
| 1145 | A = I; Z = Cl; Z′ = Cl | A = CF₂CF₂CF₃; Z = Cl; Z′ = Cl |
| 1146 | A = Br; Z = Cl; Z = Cl | A = CF₂CF₃; Z = Cl; Z′ = Cl |
| 1147 | A = I; Z = Br; Z′ = Br | A = CF₂CF₂CF₃; Z = Br; Z′ = Br |
| 1148 | A = I; Z = CO₂C₂H₅; | A = CF₂CF₂CF₃; Z = CO₂C₂H₅; |
| | Z′ = Cl | Z′ = Cl |
| 1149 | A = I; Z = Cl; Z′ = | A = CF₂CF₂CF₃; Z = Cl; Z′ = |
| | CO₂C₂H₅ | CO₂C₂H₅ |
| 1150 | A = I; Z = O—Bz; Z′ = Cl | A = CF₂CF₂CF₃; Z = O—Bz; Z′ = Cl |
| 1151 | A = I; Z = Cl; Z′ =O—Bz | A = CF₂CF₂CF₃; Z = Cl; Z′ = O—Bz |
| 1152 | A = I; Z = Br; Z′ = | A = CF₂CF₂CF₃; Z = Br; Z′ = |
| | NH—BOC | NH—BOC |
| 1153 | A = I; Z = Cl; Z = | A = CF₂CF₂CF₃; Z = Cl; Z′ = |
| | NH—BOC | NH—BOC |
| 1154 | A = I; Z = NH—BOC; | A = CF₂CF₂CF₃; Z = NH—BOC; |
| | Z′ = Br | Z′ = Br |

### Examples 1155-1164: 2-Nonafluorobutyl-5,6-Disubstituted Pyrazines

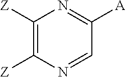

Procedure C is used to prepare the nonafluorobutyl derivative from the corresponding iodide or bromide.

| Example | Starting material | Product |
|---|---|---|
| 1155 | A = I; Z = Cl; Z′ = Cl | A = CF₂CF₂CF₂CF₃; Z = Cl; Z′ = Cl |
| 1156 | A = Br; Z = Cl; Z′ = Cl | A = CF₂CF₃; Z = Cl; Z′ = Cl |
| 1157 | A = I; Z = Br; Z′ = Br | A = CF₂CF₂CF₂CF₃; Z = Br; Z′ = Br |
| 1158 | A = I; Z = CO₂C₂H₅; | A = CF₂CF₂CF₂CF₃; Z = CO₂C₂H₅; |
| | Z′ = Cl | Z = Cl |
| 1159 | A = I; Z = Cl; Z′ = | A = CF₂CF₂CF₂CF₃; Z = Cl; Z′ = |
| | CO₂C₂H₅ | CO₂C₂H |
| 1160 | A = I; Z = O—Bz; | A = CF₂CF₂CF₂CF₃; Z = O—Bz; |
| | Z′ = Cl | Z′ = Cl |
| 1161 | A = I; Z = Cl; Z′ = | A = CF₂CF₂CF₂CF₃; Z = Cl; Z′ = |
| | O—Bz | O—Bz |
| 1162 | A = I; Z = Br; Z′ = | A = CF₂CF₂CF₂CF₃; Z = Br; Z′ = |
| | NH—BOC | NH—BOC |
| 1163 | A = I; Z = Cl; Z′ = | A = CF₂CF₂CF₂CF₃; Z = Cl; Z′ = |
| | NH—BOC | NH—BOC |
| 1164 | A = I; Z = NH—BOC; | A = CF₂CF₂CF₂CF₃; Z = NH—BOC; |
| | Z′ = Br | Z′ = Br |

### Examples 1165-1174: 2-Difluoromethyl-5,6-Disubstituted Pyrazines

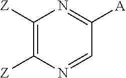

Procedure D is used to prepare the difluoromethyl derivative from the corresponding iodide or bromide.

| Example | Starting material | Product |
|---|---|---|
| 1165 | A = I; Z = Cl; Z′ = Cl | A = CHF₂; Z = Cl; Z′ = Cl |
| 1166 | A = Br; Z = Cl; Z′ = Cl | A = CF₂CF₃; Z = Cl; Z′ = Cl |
| 1167 | A = I; Z = Br; Z′ = Br | A = CHF₂; Z = Br; Z′ = Br |
| 1168 | A = I; Z = CO₂C₂H₅; Z′ = Cl | A = CHF₂; Z = CO₂C₂H₅; Z′ = Cl |
| 1169 | A = I; Z = Cl; Z′ = CO₂C₂H₅ | A = CHF₂; Z = Cl; Z′ = CO₂C₂H₅ |
| 1170 | A = I; Z = O—Bz; Z′ = Cl | A = CHF₂; Z = O—Bz; Z′ = Cl |
| 1171 | A = I; Z = Cl; Z′ = O—Bz | A = CHF₂; Z = Cl; Z′ = O—Bz |
| 1172 | A = I; Z = Br; Z′ = | A = CHF₂; Z = Br; Z′ = |
| | NH—BOC | NH—BOC |
| 1173 | A = I; Z = Cl; Z′ = | A = CHF₂; Z = Cl; Z′ = |
| | NH—BOC | NH—BOC |
| 1174 | A = I; Z = NH—BOC; | A = CHF₂; Z = NH—BOC; |
| | Z′ = Br | Z′ = Br |

### Examples 1175-1184: 2-Heptafluoroisopropyl-5,6-Disubstituted Pyrazines

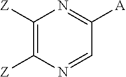

Procedure E is used to prepare the heptafluoroisopropyl propyl derivative from the corresponding iodide or bromide.

| Example | Starting material | Product |
|---|---|---|
| 1175 | A = I; Z = Cl; Z′ = Cl | A = CF(CF₃)₂; Z = Cl; Z′ = Cl |
| 1176 | A = Br; Z = Cl; Z′ = Cl | A = CF₂CF₃; Z = Cl; Z′ = Cl |
| 1177 | A = I; Z = Br; Z′ = Br | A = CF(CF₃)₂; Z = Br; Z′ = Br |
| 1178 | A = I; Z = CO₂C₂H₅; | A = CF(CF₃)₂; Z = CO₂C₂H₅; |
| | Z′ = Cl | Z′ = Cl |
| 1179 | A = I; Z = Cl; Z′ = | A = CF(CF₃)₂; Z = Cl; Z′ = |
| | CO₂C₂H₅ | CO₂C₂H₅ |
| 1180 | A = I; Z = O—Bz; Z′ = Cl | A = CF(CF₃)₂; Z = O—Bz; Z′ = Cl |
| 1181 | A = I; Z = Cl; Z′ = O—Bz | A = CF(CF₃)₂; Z = Cl; Z′ = O—Bz |
| 1182 | A = I; Z = Br; Z′ = | A = CF(CF₃)₂; Z = Br; Z′ = |
| | NH—BOC | NH—BOC |
| 1183 | A = I; Z = Cl; Z′ = | A = CF(CF₃)₂; Z = Cl; Z′ = |
| | NH—BOC | NH—BOC |
| 1184 | A = I; Z = NH—BOC; | A = CF(CF₃)₂; Z = NH—BOC; |
| | Z′ = Br | Z′ = Br |

A number of examples and embodiments of the invention have been presented, and the features and advantages of the invention will be apparent to the skilled person based on this description. Other advantages, variations, and modifications will also be evident to the skilled person, without departing from the invention. For example, in addition to the heterocycles described above, other substituted pyridines bearing a higher order fluoroalkyl group (perfluoroethyl, perfluoropropyl, perfluoroisopropyl or perfluorobutyl) or a difluoromethyl group can he prepared using the methods described herein. Examples include compounds having the following formulas:

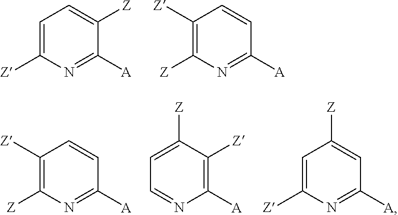

where A, Z, and Z′ are as described above.

As a second example of variations within the scope of the invention, salts—including pharmaceutically acceptable salts—of the many compounds described herein can be prepared using common techniques known to organic and medicinal chemists. Such techniques include acid addition, adjusting the pH of a solution containing the substituted heterocycle and introducing an appropriate counterion, and so forth. In general, salt formation involves the acidic or basic groups present in the fluoroalkyl-substituted heterocyclic compounds described herein, for example the aryl ring nitrogen atom(s). Acid addition salts include, but are not limited, to acid phosphate, acetate, adipate, ascorbate, benzensulfonate, benzoate, bisulfate, bitartrate, citrate, formate, fumarate, ethanesulfonate, gentisinate, gluconate, gluacaronate, glutamate, glutarate, hydrobromide, hydrochloride, hydroiodide, isonicotinate, lactate, maleate, methanesulfonate, oxalate, nitrate, pamoate, pantothenate, phosphate, phosphonate, saccharate, salicylate, succinate, sulfate, tartrate, and p-toluenesulfonate salts. Pharmaceutically acceptable salts are reviewed in BERGE ET AL., 66 J. PHARM. SCI. 1-19 (1977), incorporated herein by reference. A more recent list is found in P. H. Stahl and C. G. Wermuth, editors, Handbook of Pharmaceutical salts; Properties, Selection and Use, Weinheim/Zurich:Wiley-VCH/VCHA, 2002, incorporated herein by reference. All such variations and modifications that would be apparent to a skilled person after reading the instant disclosure fall within the scope of the invention, which is limited only by the appended claims and equivalents thereof.

What is claimed is:

1. A fluoroalkyl-substituted derivative of pyridine, pyrimidine, or pyrazine, having any of the following formulas: 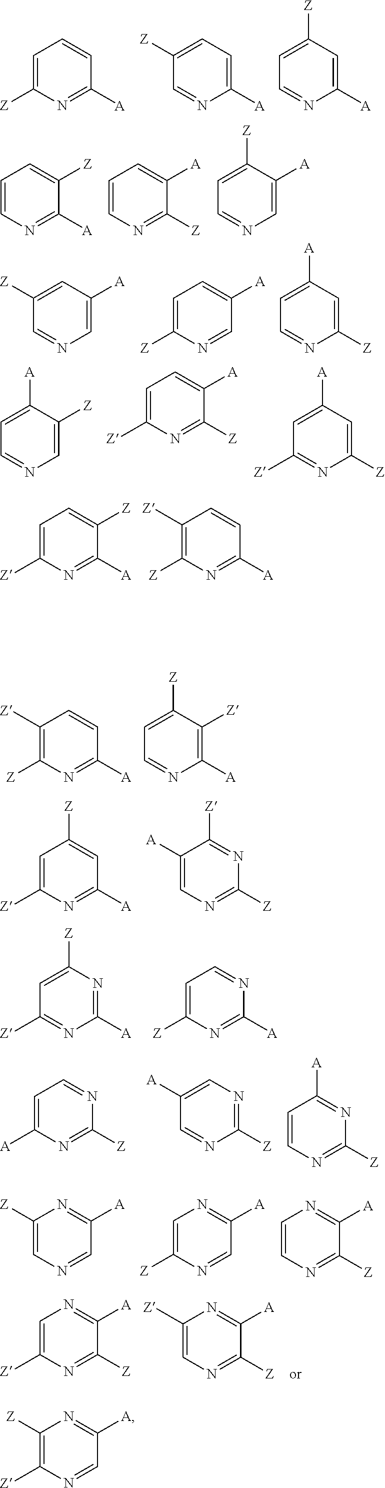
where A is selected from the group consisting of CF₂CF₃, CF₂CF₂CF₃, CF(CF₃)₂, and CF₂CF₂CF₂CF₃; and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO₂, NHBOC (where BOC is t-butoxycarbonyl), and NH₂, provided that (a) if the derivative has the formula 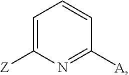
where A is CF₂CF₃, then Z is not Br or CN,
(b) if the derivative has the formula 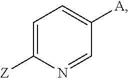
where A is CF₂CF₃, then Z is not Cl, and
(c) if the derivative has the formula 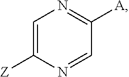
where A is CF₂CF₃, then Z is not Br.

2. A salt of a fluoroalkyl-substituted derivative of pyridine, pyrimidine, or pyrazine as recited in claim 1.

3. A salt as recited in claim 2, wherein the salt is a pharmaceutically acceptable salt.

4. A fluoroalkyl-substituted pyridine derivative, having any of the following formulas: 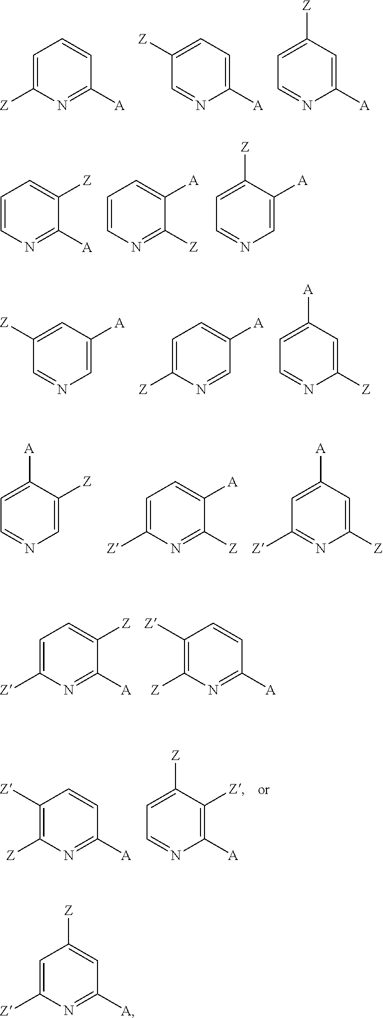
where A is selected from the group consisting of CF₂CF₃, CF₂CF₂CF₃, CF(CF₃)₂, and CF₂CF₂CF₂CF₃; and Z and Z′ are independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO₂, NHBOC (where BOC is t-butoxycarbonyl), and NH₂, provided that (a) if the pyridine derivative has the formula 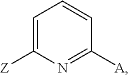
where A is CF₂CF₃, then Z is not Br or CR and
b) if the derivative has the formula 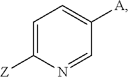
where A is CF₂CF₃, then Z is not Cl.

5. A fluoroalkyl-substituted pyrimidine derivative, having any of the following formulas: 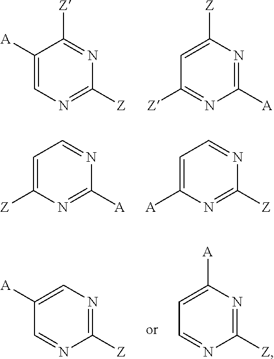
where A is selected from the group consisting of CF₂CF₃, CF₂CF₂CF₃, CF(CF₃)₂, and CF₂CF₂CF₂CF₃; and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO, NHBOC (where BOC is t-butoxycarbonyl), and NH₂.

6. A fluoroalkyl-substituted pyrazine derivative, having any of the following formulas: 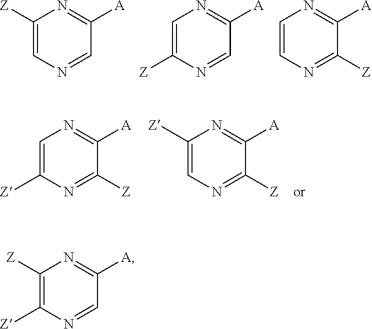
where A is selected from the group consisting of CF₂CF₃, CF₂CF₂ CF₃, CF(CF₃)₂, and CF₂CF₂CF₂CF₃; and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO₂, NHBOC (where BOC is t-butoxycarbonyl), and NH₂, provided that if the derivative has the formula 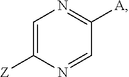
where A is CF₂CF₃, then Z is not Br.

7. A fluoroalkyl-substituted derivative of pyridine, pyrimidine, or pyrazine, having any of the following formulas: 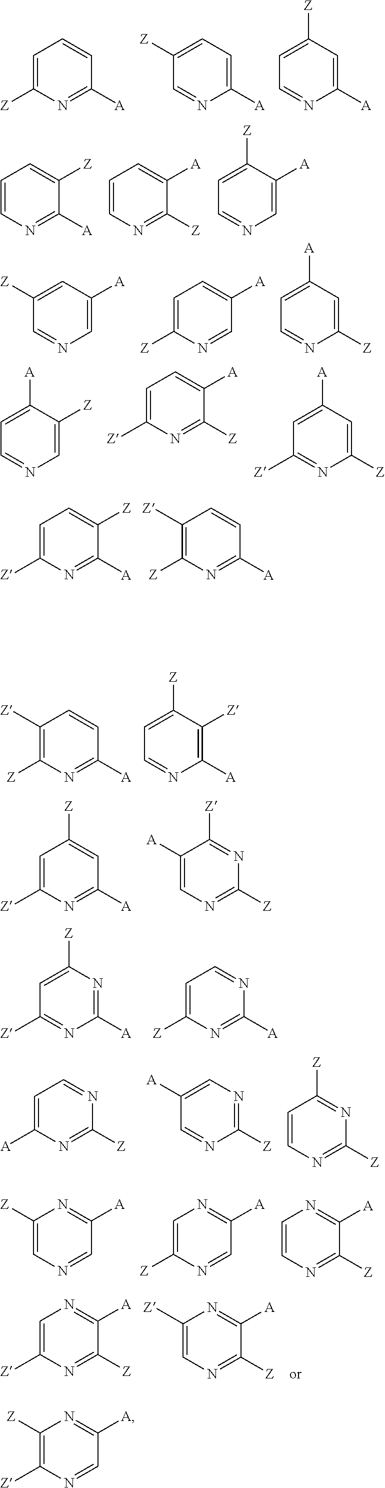
where A is CHF₂ and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, ON, Spin (where Bpin is pinacol boronate), BMIDA (where BMIDA is Boron-N-methyl-iminodiacetic acid complex), NO₂, NHBOC (where BOC is t-butoxycarbonyl), and NH₂.

8. A salt of a fluoroalkyl-substituted derivative of pyridine, pyrimidine, or pyraxine as recited in claim 7.

9. A salt as recited in claim 8, wherein the salt is a pharmaceutically acceptable salt.

10. A fluoroalkyl-substituted pyridine derivative, having any of the following formulas: 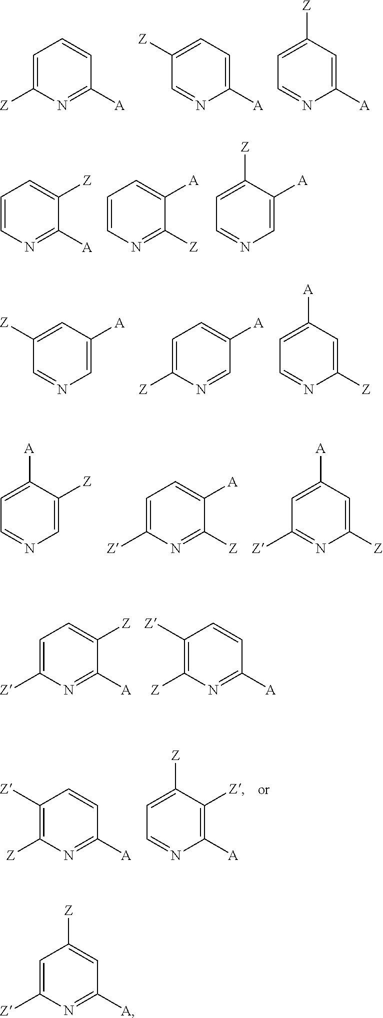
where A is CHF₃ and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO₂, NHBOC (where BOC is t-butoxycarbonyl), and NH₂.

11. A fluoroalkyl-substituted pyrimidine derivative, having any of the following formulas: 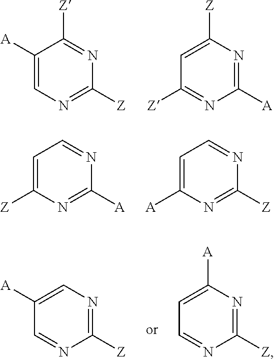
where A is CHF₂ and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO₂, NHBOC (where BOC is t-butoxycarbonyl), and NH₂.

12. A fluoroalkyl-substituted pyrazine derivative, having any of the following formulas: 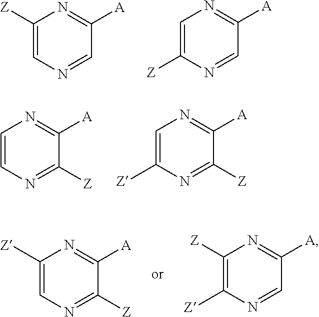
where A is CHF₂ and Z and Z′ are, independently, selected from the group consisting of Cl, CO₂C₂H₅, CO₂CH₃, OC₂H₅, CONH₂, COCH₃, CONH₂, CHO, OBz (where Bz is benzyl), Br, CN, Bpin (where Bpin is pinacol boronate), BMIDA (where BMIDA is Boron-N-methyl-iminodiacetic acid complex), NO₂, NHBOC (where BOC is t-butoxycarbonyl), and NH₂.
